SEQUENCE LISTING (53 sequences)

SEQ ID NO: 1 - protein, 245 aa, synthetic construct
EVQLVESGGGLIQPGGSLRLSCAASGFTFSTYAMSWVRQAPGKGLEWVSSISSGGNTYYADSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIK

SEQ ID NO: 2 - protein, 121 aa, synthetic construct
EVQLVESGGGLIQPGGSLRLSCAASGFTFSTYAMSWVRQAPGKGLEWVSSISSGGNTYYADSVKGRFTISRDNSKNTLYLQMNSLRAEDTAVYYCARDSYYFGNSVYYAMDYWGAGTTVTV

SEQ ID NO: 3 - protein, 107 aa, synthetic construct
DIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIK

SEQ ID NO: 4 - protein, 501 aa, synthetic construct
MALPVTALLLPLALLLHAARPASEVQLVESGGGLIQPGGSLRLSCAASGFTFSTYAMSWVRQAPGKGLEWVSSISSGGNTYYADSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIKLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 245 aa, synthetic construct
QVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIK

SEQ ID NO: 6 - protein, 123 aa, synthetic construct
QVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSS

SEQ ID NO: 7 - protein, 107 aa, synthetic construct; identical to SEQ ID NO: 3

SEQ ID NO: 8 - protein, 501 aa, synthetic construct
MALPVTALLLPLALLLHAARPASQVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWFQQKPGKAPKSLIYRANRLVSGVPSRFSGSGSGTDFTLTISSLQPEDFATYYCLQYDEFPYTFGGGTRLEIKLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 245 aa, synthetic construct
QVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIK

SEQ ID NO: 10 - protein, 123 aa, synthetic construct; identical to SEQ ID NO: 6

SEQ ID NO: 11 - protein, 107 aa, synthetic construct
DIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIK

SEQ ID NO: 12 - protein, 501 aa, synthetic construct
MALPVTALLLPLALLLHAARPASQVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIKLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - DNA, 735 bp, synthetic construct
  source 1..735: mol_type = other DNA
gaagtacagcttgttgaatcaggtggtggtcttattcagccaggaggctccttgcgactgagctgtgccgcttctgggttcacctttagcacttacgcaatgagttgggtccgacaagccccaggtaagggattggaatgggtaagttccatttccagcggagggaacacttattacgccgattctgtgaaaggacgctttactatatcccgagacaatagtaaaaacacattgtatttgcaaatgaactctttgagggccgaggacactgccgtctactattgtgcccgcgacagctattatttcggcaactctgtgtattacgcgatggattactggggtgccggcacaactgtcaccgtttcatctggcggaggaggcagtggcggagggggctcaggcggtggtggaagtgatattcaaatgacccaatcaccctcttcattgtctgcaagcgtaggtgaccgagtcacgataacctgcaaagcctctcaagatattaattcatacttttcttggtttcaacaaaaaccgggaaaggcgcctaagtcattgatttaccgcgcgaaccggttggtatcaggagtaccgtcaagattctcagggagtgggtcaggcacagatttcacactcactatttcttccttgcaacctgaagacttcgcaacctattattgcttgcagtatgatgagtttccgtacactttcggggggggtacaaggctggagatcaaa

SEQ ID NO: 14 - DNA, 1506 bp, synthetic construct
  source 1..1506: mol_type = other DNA
atggccttaccagtgaccgccttgctcctgccgctggccttgctgctccacgccgccaggccggctagcgaagtacagcttgttgaatcaggtggtggtcttattcagccaggaggctccttgcgactgagctgtgccgcttctgggttcacctttagcacttacgcaatgagttgggtccgacaagccccaggtaagggattggaatgggtaagttccatttccagcggagggaacacttattacgccgattctgtgaaaggacgctttactatatcccgagacaatagtaaaaacacattgtatttgcaaatgaactctttgagggccgaggacactgccgtctactattgtgcccgcgacagctattatttcggcaactctgtgtattacgcgatggattactggggtgccggcacaactgtcaccgtttcatctggcggaggaggcagtggcggagggggctcaggcggtggtggaagtgatattcaaatgacccaatcaccctcttcattgtctgcaagcgtaggtgaccgagtcacgataacctgcaaagcctctcaagatattaattcatacttttcttggtttcaacaaaaaccgggaaaggcgcctaagtcattgatttaccgcgcgaaccggttggtatcaggagtaccgtcaagattctcagggagtgggtcaggcacagatttcacactcactatttcttccttgcaacctgaagacttcgcaacctattattgcttgcagtatgatgagtttccgtacactttcggggggggtacaaggctggagatcaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 15 - DNA, 731 bp, synthetic construct
  source 1..731: mol_type = other DNA
caggtacaattggtagagtccggcggaggggttgttcagccaggacggtcttgcgttgtcttgtgctgcgtcaggattcacattctcaacgtacgcgatgtcttgggtgcgccaagctcccggtaaagggctggaatgggtggcctcaatctcatctggagggaacacttactaccctgatagtgttaaaggtcgctttactatctcaagggacaatagcaagaataccttgtatctgcaaatgaactcacttagagcagaggacacagcggtatattactgtgctagagactcatattatttcggcaactccgtttattacgcgatggattactggggcgcagggactacggtaactgtatcttctggtggtggagggtctgggggcgggggtagtggcggcggtggcagtgacatccagatgacacagtctccgtcttcattgagtgcaagcgtcggcgatcgggttaccattacgtgtaaggcaagtcaggacatcaacagttatttttcatggtttcaacaaaagcctggaaaagcgccgaaatcactcatttaccgagctaataggcttgtctctggcgttccgtctcgcttcagtggaagtgggagcggtactgattttaccctcaccatatcaagcctcaaccggaggattttgcccgtactattgtctccagtacgatgaatttccatatacgtttggcggcgggactcgcttggagattaaa

SEQ ID NO: 16 - DNA, 1506 bp, synthetic construct
  source 1..1506: mol_type = other DNA
atggccttaccagtgaccgccttgctcctgccgctggccttgctgctccacgccgccaggccggctagccaggtacaattggtagagtccggcggaggggttgttcagccaggacggtccttgcggttgtcttgtgctgcgtcaggattcacattctcaacgtacgcgatgtcttgggtgcgccaagctcccggtaaagggctggaatgggtggcctcaatctcatctggagggaacacttactaccctgatagtgttaaaggtcgctttactatctcaagggacaatagcaagaataccttgtatctgcaaatgaactcacttagagcagaggacacagcggtatattactgtgctagagactcatattatttcggcaactccgtttattacgcgatggattactggggcgcagggactacggtaactgtatcttctggtggtggagggtctgggggcgggggtagtggcggcggtggcagtgacatccagatgacacagtctccgtcttcattgagtgcaagcgtcggcgatcgggttaccattacgtgtaaggcaagtcaggacatcaacagttatttttcatggtttcaacaaaagcctggaaaagcgccgaaatcactcatttaccgagctaataggcttgtctctggcgttccgtctcgcttcagtggaagtgggagcggtactgattttaccctcaccatatcaagccttcaaccggaggattttgccacgtactattgtctccagtacgatgaatttccatatacgtttggcggcgggactcgcttggagattaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - DNA, 735 bp, synthetic construct
  source 1..735: mol_type = other DNA
caggtacaactggtggaatccggcgggggagtagtacagcccggacgatctcttcgactctcatgtgcagcgtccgggttcactttttctacctacgcaatgtcatgggtacgacaggcgccgggcaaaggcctcgaatgggttgcatccatttcatcaggaggtaatacatattatcctgattcagtcaagggccgattcacgattagtcgagataatagcaagaacactctctacttgcagatgaactccctgcgggctgaggacacggccgtgtattattgcgctcgcgatagttattacttcggcaattccgtatattatgcgatggactattggggcgccggtactaccgtgactgtttcctctggtgggggtgggtccgggggcggtggttcaggtggaggcggatccgacattcaaatgacccagtctccctcaagtttgtctgcatctgttggcgatagagttacaataacatgcaaagccagtcaagacatcaactcatacttctcctggtatcaacaaaagccaggaaaagctccgaaactgttgatctaccgggccaaccggctggtcactggcgtgccatcccggttcagtggcagcggaagcggaacagatttcacgtttaccatctctagcctccaaccggaggacatcgcaacatactattgccttcagtatgatgagtttccctacactttcggtggcggcacccgacttgagatcaaa

SEQ ID NO: 18 - DNA, 1506 bp, synthetic construct
  source 1..1506: mol_type = other DNA
atggccttaccagtgaccgccttgctcctgccgctggccttgctgctccacgccgccaggccggctagccaggtacaactggtggaatccggcgggggagtagtacagcccggacgatctcttcgactctcatgtgcagcgtccgggttcactttttctacctacgcaatgtcatgggtacgacaggcgccgggcaaaggcctcgaatgggttgcatccatttcatcaggaggtaatacatattatcctgattcagtcaagggccgattcacgattagtcgagataatagcaagaacactctctacttgcagatgaactccctgcgggctgaggacacggccgtgtattattgcgctcgcgatagttattacttcggcaattccgtatattatgcgatggactattggggcgccggtactaccgtgactgtttcctctggtgggggtgggtccgggggcggtggttcaggtggaggcggatccgacattcaaatgacccagtctccctcaagtttgtctgcatctgttggcgatagagttacaataacatgcaaagccagtcaagacatcaactcatacttctcctggtatcaacaaaagccaggaaaagctccgaaactgttgatctaccgggccaaccggctggtcactggcgtgccatcccggttcagtggcagcggaagcggaacagatttcacgtttaccatctctagcctccaaccggaggacatcgcaacatactattgccttcagtatgatgagtttccctacactttcggtggcggcacccgacttgagatcaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 19 - DNA, 141 bp, Homo sapiens
  source 1..141: mol_type = genomic DNA
aagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgat

SEQ ID NO: 20 - protein, 47 aa, Homo sapiens
KPTTTPAPRPPTPAPTIASQPLSLRPEASRPAAGGAVHTRGLDFASD

SEQ ID NO: 21 - DNA, 204 bp, Homo sapiens
  source 1..204: mol_type = genomic DNA
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 22 - protein, 68 aa, Homo sapiens
FWVLVVVGGVLACYSLLVTVAFIIFWVRSKRSRLLHSDYMNMTPRRPGPTRKHYQPYAPPRDFAAYRS

SEQ ID NO: 23 - DNA, 339 bp, Homo sapiens
  source 1..339: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 24 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 26 - protein, 42 aa, Homo sapiens
KRGRKKLLYIFKQPFMRPVQTTQEEDGCSCRFPEEEEGGCEL

SEQ ID NO: 27 - protein, 235 aa, synthetic construct
QVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSDIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIK

SEQ ID NO: 28 - DNA, 705 bp, synthetic construct
  source 1..705: mol_type = other DNA
caggtacaactggtggaatccggcgggggagtagtacagcccggacgatctcttcgactctcatgtgcagcgtccgggttcactttttctacctacgcaatgtcatgggtacgacaggcgccgggcaaaggcctcgaatgggttgcatccatttcatcaggaggtaatacatattatcctgattcagtcaagggccgattcacgattagtcgagataatagcaagaacactctctacttgcagatgaactccctgcgggctgaggacacggccgtgtattattgcgctcgcgatagttattacttcggcaattccgtatattatgcgatggactattggggcgccggtactaccgtgactgtttcctctggtggaggcggatccgacattcaaatgacccagtctccctcaagtttgtctgcatctgttggcgatagagttacaataacatgcaaagccagtcaagacatcaactcatacttctcctggtatcaacaaaagccaggaaaagctccgaaactgttgatctaccgggccaaccggctggtcactggcgtgccatcccggttcagtggcagcggaagcggaacagatttcacgtttaccatctctagcctccaaccggaggacatcgcaacatactattgccttcagtatgatgagtttccctacactttcggtggcggcacccgacttgagatcaaa

SEQ ID NO: 29 - skipped

SEQ ID NO: 30 - protein, 6 aa, synthetic construct
SSGGNT

SEQ ID NO: 31 - protein, 15 aa, synthetic construct
DSYYFGNSVYYAMDY

SEQ ID NO: 32 - protein, 6 aa, synthetic construct
QDINSY

SEQ ID NO: 33 - skipped

SEQ ID NO: 34 - protein, 9 aa, synthetic construct
LQYDEFPYT

SEQ ID NO: 35 - protein, 489 aa, synthetic construct
MALPVTALLLPLALLLHAARPQVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSGGGGSGGGGSDIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 1467 bp, synthetic construct
  source 1..1467: mol_type = other DNA
atggcgttgcccgtgaccgcacttctgcttccattggcactcctgctccacgcggcaagaccgcaggtacaactggtggaatccggcgggggagtagtacagcccggacgatctcttcgactctcatgtgcagcgtccgggttcactttttctacctacgcaatgtcatgggtacgacaggcgccgggcaaaggcctcgaatgggttgcatccatttcatcaggaggtaatacatattatcctgattcagtcaagggccgattcacgattagtcgagataatagcaagaacactctctacttgcagatgaactccctgcgggctgaggacacggccgtgtattattgcgctcgcgatagttattacttcggcaattccgtatattatgcgatggactattggggcgccggtactaccgtgactgtttcctctggtgggggtgggtccgggggcggtggttcaggtggaggcggatccgacattcaaatgacccagtctccctcaagtttgtctgcatctgttggcgatagagttacaataacatgcaaagccagtcaagacatcaactcatacttctcctggtatcaacaaaagccaggaaaagctccgaaactgttgatctaccgggccaaccggctggtcactggcgtgccatcccggttcagtggcagcggaagcggaacagatttcacgtttaccatctctagcctccaaccggaggacatcgcaacatactattgccttcagtatgatgagtttccctacactttcggtggcggcacccgacttgagatcaaaacaaccacgcctgcccctcggccacccacgcctgcacccactatcgcttcccagccactctctcttcggccggaggcttgtcgccccgcagcgggaggcgcggttcatactcgcgggctggactttgcttgcgacatctacatctgggcaccgcttgccggaacgtgcggggtcttgctgctgtccctcgttattactctttactgcaaaagaggaagaaaaaagttgctgtatatttttaagcaaccatttatgcgcccggtccaaactacgcaagaggaggatggatgtagctgccgattccccgaagaagaggagggtggttgcgaactgagagtgaaatttagccggtctgctgacgctccggcctatcagcaagggcaaaaccaactttacaatgagcttaacctggggaggcgagaggaatatgatgtattggataagcgccgagggagggacccagagatgggaggaaaaccgaggagaaaaaacccgcaagaggggctttataatgaactgcagaaagataagatggcggaggcttacagcgagatcgggatgaagggagagagacgcagagggaaaggccacgacggtctctaccaaggcctgagtacggccacgaaagatacatacgatgccctccatatgcaggccctgccaccgagg

SEQ ID NO: 37 - protein, 479 aa, synthetic construct
MALPVTALLLPLALLLHAARPQVQLVESGGGVVQPGRSLRLSCAASGFTFSTYAMSWVRQAPGKGLEWVASISSGGNTYYPDSVKGRFTISRDNSKNTLYLQMNSLRAEDTAVYYCARDSYYFGNSVYYAMDYWGAGTTVTVSSGGGGSDIQMTQSPSSLSASVGDRVTITCKASQDINSYFSWYQQKPGKAPKLLIYRANRLVTGVPSRFSGSGSGTDFTFTISSLQPEDIATYYCLQYDEFPYTFGGGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 1437 bp, synthetic construct
  source 1..1437: mol_type = other DNA
atggcgttgcccgtgaccgcacttctgcttccattggcactcctgctccacgcggcaagaccgcaggtacaactggtggaatccggcgggggagtagtacagcccggacgatctcttcgactctcatgtgcagcgtccgggttcactttttctacctacgcaatgtcatgggtacgacaggcgccgggcaaaggcctcgaatgggttgcatccatttcatcaggaggtaatacatattatcctgattcagtcaagggccgattcacgattagtcgagataatagcaagaacactctctacttgcagatgaactccctgcgggctgaggacacggccgtgtattattgcgctcgcgatagttattacttcggcaattccgtatattatgcgatggactattggggcgccggtactaccgtgactgtttcctctggtggaggcggatccgacattcaaatgacccagtctccctcaagtttgtctgcatctgttggcgatagagttacaataacatgcaaagccagtcaagacatcaactcatacttctcctggtatcaacaaaagccaggaaaagctccgaaactgttgatctaccgggccaaccggctggtcactggcgtgccatcccggttcagtggcagcggaagcggaacagatttcacgtttaccatctctagcctccaaccggaggacatcgcaacatactattgccttcagtatgatgagtttccctacactttcggtggcggcacccgacttgagatcaaaacaaccacgcctgcccctcggccacccacgcctgcacccactatcgcttcccagccactctctcttcggccggaggcttgtcgccccgcagcgggaggcgcggttcatactcgcgggctggactttgcttgcgacatctacatctgggcaccgcttgccggaacgtgcggggtcttgctgctgtccctcgttattactctttactgcaaaagaggaagaaaaaagttgctgtatatttttaagcaaccatttatgcgcccggtccaaactacgcaagaggaggatggatgtagctgccgattccccgaagaagaggagggtggttgcgaactgagagtgaaatttagccggtctgctgacgctccggcctatcagcaagggcaaaaccaactttacaatgagcttaacctggggaggcgagaggaatatgatgtattggataagcgccgagggagggacccagagatgggaggaaaaccgaggagaaaaaacccgcaagaggggctttataatgaactgcagaaagataagatggcggaggcttacagcgagatcgggatgaagggagagagacgcagagggaaaggccacgacggtctctaccaaggcctgagtacggccacgaaagatacatacgatgccctccatatgcaggccctgccaccgagg

SEQ ID NO: 39 - skipped

SEQ ID NO: 40 - protein, 4 aa, Homo sapiens
YRHQ

SEQ ID NO: 41 - skipped

SEQ ID NO: 42 - protein, 4 aa, Homo sapiens
YLSL

SEQ ID NO: 43 - protein, 9 aa, Homo sapiens
LKCNTPDPS

SEQ ID NO: 44 - protein, 172 aa, synthetic construct
MYRMQLLSCIALSLALVTNSSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 45 - DNA, 516 bp, synthetic construct
  source 1..516: mol_type = other DNA
atgtaccgcatgcaacttctgtcttgtattgcgctttctctcgcactcgtaaccaattccagcatgtgtaagccgataaccgggactataaacgatttgaatcagcaggtatggaccttgcagggacagaatctggtagctgtaccacggagcgacagcgtcacccccgtgactgtggccgtcataacatgcaaatacccggaagcgctcgaacagggcaggggtgatccgatatacctgggaatccaaaacccggagatgtgcctttactgcgagaaggtgggcgaacaacctacgcttcaattgaaagagcaaaagataatggacctgtacggccaacccgaacccgtaaagcccttcctcttttaccgcgccaagactggtagaacaagtactctggagagcgttgcttttcctgattggtttatagcaagctccaaaagggaccagccgattatcctcacaagcgaactcggaaagtcctataataccgcttttgagttgaatatcaatgac

SEQ ID NO: 46 - protein, 169 aa, synthetic construct
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 47 - DNA, 507 bp, synthetic construct
  source 1..507: mol_type = other DNA
atgagagggactcccggagacgcagacggtggaggccgagctgtttatcaaagcatgtgtaagccgataaccgggactataaacgatttgaatcagcaggtatggaccttgcagggacagaatctggtagctgtaccacggagcgacagcgtcacccccgtgactgtggccgtcataacatgcaaatacccggaagcgctcgaacagggcaggggtgatccgatatacctgggaatccaaaacccggagatgtgcctttactgcgagaaggtgggcgaacaacctacgcttcaattgaaagagcaaaagataatggacctgtacggccaacccgaacccgtaaagcccttcctcttttaccgcgccaagactggtagaacaagtactctggagagcgttgcttttcctgattggtttatagcaagctccaaaagggaccagccgattatcctcacaagcgaactcggaaagtcctataataccgcttttgagttgaatatcaatgac

SEQ ID NO: 48 - protein, 172 aa, synthetic construct
MRRMQLLLLIALSLALVTNSSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 49 - DNA, 516 bp, synthetic construct
  source 1..516: mol_type = other DNA
atgcggcgcatgcaacttctgctgctcattgcgctttctctcgcactcgtaaccaattccagcatgtgtaagccgataaccgggactataaacgatttgaatcagcaggtatggaccttgcagggacagaatctggtagctgtaccacggagcgacagcgtcacccccgtgactgtggccgtcataacatgcaaatacccggaagcgctcgaacagggcaggggtgatccgatatacctgggaatccaaaacccggagatgtgcctttactgcgagaaggtgggcgaacaacctacgcttcaattgaaagagcaaaagataatggacctgtacggccaacccgaacccgtaaagcccttcctcttttaccgcgccaagactggtagaacaagtactctggagagcgttgcttttcctgattggtttatagcaagctccaaaagggaccagccgattatcctcacaagcgaactcggaaagtcctataataccgcttttgagttgaatatcaatgac

SEQ ID NO: 50 - protein, 94 aa, synthetic construct
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 51 - DNA, 282 bp, synthetic construct
  source 1..282: mol_type = other DNA
aactgccgaaacaccggaccctggctcaaaaaggttcttaaatgcaacactcctgacccatccaagtttttttcacaattgtcatcagagcacggaggcgatgtacaaaagtggcttagttcccctttcccgagctcctcctttagtccagggggtctcgcccctgaaattagtcccctcgaagttttggagagagataaggtgacccaattgctcccactgaacaccgacgcatatctctccttgcaagagttgcaaggccaggaccccacccacctggtc

SEQ ID NO: 52 - protein, 5 aa, synthetic construct
GGGGS

SEQ ID NO: 53 - protein, 20 aa, synthetic construct
GGGGSGGGGSGGGGSGGGGS

(19) United States
(12) Patent Application Publication
(10) Pub. No.: US 2026/0335438 A1
(43) Pub. Date: Oct. 29, 2026

(54) ANTI-ROR1 CHIMERIC ANTIGEN RECEPTORS (CARS), CELLS EXPRESSING THE CARS AND RELATED METHODS

(71) Applicant: Caribou Biosciences, Inc., Berkeley, CA (US)

(72) Inventors: Emilie Degagne, Berkeley, CA (US); Paul D. Donohoue, San Francisco, CA (US); Leslie Edwards, San Ramon, CA (US); Elizabeth Garner, Novato, CA (US); Rodolfo Gonzalez, Burlingame, CA (US); Erica Hennessy, Benicia, CA (US); Steven B. Kanner, Los Angeles, CA (US); Bastien Vidal, San Bruno, CA (US)

(73) Assignee: Caribou Biosciences, Inc., Berkeley, CA (US)

(21) Appl. No.: 19/116,902

(22) PCT Filed: Sep. 28, 2023
(86) PCT No.: PCT/US2023/075395

§ 371 Date: Mar. 28, 2025

Related U.S. Application Data
(60) Provisional application No. 63/377,810, filed on Sep. 30, 2022.
(60) Provisional application No. 63/386,747, filed on Dec. 9, 2022.
(60) Provisional application No. 63/483,504, filed on Feb. 6, 2023.
(60) Provisional application No. 63/511,064, filed on Jun. 29, 2023.

(51) Int. Cl. A61K 40/31; A61K 35/17; A61K 40/11; A61K 40/22; A61K 40/42; C07K 14/705; C07K 14/715; C07K 16/28; C12N 5/0783; C12N 9/22; C12N 15/11

(52) U.S. Cl. CPC A61K 40/31; A61K 35/17; A61K 40/11; A61K 40/22; A61K 40/421; C07K 14/7051; C07K 14/70517; C07K 14/70596; C07K 14/715; C07K 16/2803; C12N 5/0636; C12N 5/0646; C12N 9/22; C12N 15/111; A61K 2239/13; A61K 2239/17; A61K 2239/21; A61K 2239/22; C07K 2317/565; C07K 2317/622; C12N 2310/20; C12N 2510/00

(57) ABSTRACT

The invention comprises anti-ROR1 chimeric antigen receptor (CAR) T cells (CAR-T cells) natural killer cells (CAR-NK cells), compositions comprising the cells and methods of making and using the same, including methods of treatment of ROR1-expressing tumors.

### FIELD OF THE INVENTION

The invention related to the field of oncology and more specifically, to cell therapy with genetically engineered tumor-targeting immune cells.

### STATEMENT REGARDING FEDERALLY SPONSORED RESEARCH

None.

### SEQUENCE LISTING

The instant application contains a Sequence Listing which has been submitted electronically in XML format and is hereby incorporated by reference in its entirety. Said XML copy, created on Aug. 29, 2023, is named CBI049_30_SL.xml and is 61,699 bytes in size.

### BACKGROUND OF THE INVENTION

Chimeric antigen receptor (CAR)-T cell therapies have demonstrated remarkable potential against hematologic malignancies.

CAR-T cells have not shown such robust and reproducible antitumor activity against solid malignancies. Natural killer (NK) cells express the receptor NKG2D promoting their innate ability to detect and kill tumors that express stress ligands. NK cells are also more active than T cells in the immunosuppressive tumor microenvironment.

ROR1 is expressed in a wide range of tumors including hematologic malignancies, ovarian, lung, pancreatic, and breast cancers. There is a need for innovative treatments for hematological and solid tumors expressing ROR1, such as treatments with engineered cells targeting tumor-specific ROR1 expression.

### SUMMARY OF THE INVENTION

In one embodiment, the invention is a chimeric antigen receptor (CAR) comprising: an anti-ROR1 scFv; a transmembrane domain; a hinge domain, and a cytoplasmic domain. In some embodiments, the cytoplasmic domain comprises a CD3 zeta domain and a 4-1BB domain. In some embodiments, the anti-ROR1 scFv comprises consists essentially of a light chain (V_{L}) and a heavy chain (V_{H}), and the V_{L} comprises consists essentially of a sequence selected from SEQ ID NOs: 3, 7, and 11, the V_{H} comprises consists essentially of a sequence selected from SEQ ID NOs: 2, 6, and 10. In some embodiments, the anti-ROR1 scFv comprises a linker linking the light chain (V_{L}) and the heavy chain (V_{H}). In some embodiments, the linker comprises a formula (GₓS_{y})ₙ, where G is glycine and S is serine, e.g., G₄S. In some embodiments, the anti-ROR1 scFv comprises complementarity determining regions CDR1, CDR2, and CDR3 in the light chain (V_{L}), and CDR1, CDR2, and CDR3 in the heavy chain (V_{H}) and comprises: SEQ ID NO: 29 in the CDR1 of the V_{H}, SEQ ID NO: 30 in the CDR2 of the V_{H}, SEQ ID NO: 31 in the CDR3 of the V_{H}, SEQ ID NO: 32 in the CDR1 of the V_{L}, SEQ ID NO: 33 in the CDR2 of the V_{L}, and SEQ ID NO: 34 in the CDR3 of the V_{L}. In some embodiments, the CDR1 of the V_{H} consists of SEQ ID NO: 29, the CDR2 of the V_{H} consists of SEQ ID NO: 30, the CDR3 of the V_{H} consists of SEQ ID NO: 31, the CDR1 of the V_{L} consists of SEQ ID NO: 32, the CDR2 of the V_{L} consists of SEQ ID NO: 33, and the CDR3 of the V_{L} consists of SEQ ID NO: 34. In some embodiments, the cytoplasmic domain comprises a CD3zeta domain. In some embodiments, the transmembrane domain comprises a CD8 transmembrane domain. In some embodiments, the CD8 transmembrane domain consists essentially of SEQ ID NO: 22. In some embodiments, the CD8 transmembrane domain is encoded by a nucleic acid consisting essentially of SEQ ID NO: 21. In some embodiments, the hinge domain comprises a CD8 hinge domain. In some embodiments, the CD8 hinge domain consists essentially of SEQ ID NO. 20. In some embodiments, the CD8 hinge domain is encoded by a nucleic acid consisting essentially of SEQ ID NO. 19. In some embodiments, the CD3 zeta domain consists essentially of SEQ ID NO. 24. In some embodiments, the CD3 zeta domain is encoded by a nucleic acid consisting essentially of SEQ ID NO. 23. In some embodiments, the 4-1BB domain consists essentially of SEQ ID NO. 26. In some embodiments, the 4-1BB domain is encoded by a nucleic acid consisting essentially of SEQ ID NO. 25. In some embodiments, the CAR further comprises a signal sequence. In some embodiments, the signal sequence is selected from a CD8 signal sequence and a CD28 signal sequence. In some embodiments, the cytoplasmic domain comprises a binding motif for an intracellular signal transduction protein. In some embodiments, the binding motif is present in the CD3zeta domain. In some embodiments, the intracellular signal transduction protein is a STAT protein or a JAK protein. In some embodiments, the JAK binding domain comprises SEQ ID NO: 43. In some embodiments, the STAT binding domain is selected from SEQ ID NO: 39-42. In some embodiments, the cytoplasmic domain further comprises an IL-2Rb cytoplasmic domain. In some embodiments, the IL-2Rb cytoplasmic domain consists essentially of SEQ ID NO: 50. In some embodiments, the IL-2Rb cytoplasmic domain is encoded by a nucleic acid consisting essentially of SEQ ID NO. 51.

In some embodiments, the CAR comprises or consists essentially of the sequence selected from SEQ ID NOs: 4, 8, 12, and 27. In some embodiments, the CAR is encoded by the nucleic acid comprising or consisting essentially of the sequence selected from SEQ ID NOs: 14, 16, 18, 36, and 38.

In one embodiment, the invention is an isolated nucleic acid comprising a vector sequence and a sequence encoding the chimeric antigen receptor (CAR) described herein. In some embodiments, the isolated nucleic acid further comprises a promoter selected from the group consisting of PGK1 promoter, MND promoter, Ubc promoter, CAG promoter, CaMKIIa promoter, SV40 early promoter, SV40 late promoter, the cytomegalovirus (CMV) immediate early promoter, Rous sarcoma virus long terminal repeat (RSV-LTR) promoter, mouse mammary tumor virus long terminal repeat (MMTV-LTR) promoter, β-interferon promoter, the hsp70 promoter EF-1α promoter, and β-Actin promoter. In some embodiments, the promoter comprises a CAG promoter. In some embodiments, the promoter comprises an MND promoter. In some embodiments, the promoter comprises an EF-1α promoter. In some embodiments, the vector comprises a plasmid. In some embodiments, the vector comprises a viral vector derived from a virus selected from the group consisting of an adenovirus type 2 and an adenovirus type 5, a retrovirus, a lentivirus, an adeno-associated virus (AAV), a simian virus 40 (SV-40), vaccinia virus, Sendai virus, Epstein-Barr virus (EBV), and herpes simplex virus (HSV). In some embodiments, the isolated nucleic acid further comprises a coding sequence for a cytokine. In some embodiments, the cytokine is IL-36gamma. In some embodiments, IL-36gamma is encoded by a nucleic acid comprising a sequence selected from SEQ ID NOs 44, 46, and 48. In some embodiments, the isolated nucleic acid comprises a sequence selected from SEQ ID NOs: 14, 16, 18, 36, and 38.

In one embodiment, the invention is an immune cell comprising the chimeric antigen receptor (CAR) described herein. In some embodiments, the cell is selected from a T cell, a natural killer (NK) cell and an induced natural killer (iNK) cell. In some embodiments, the comprises a sequence selected from SEQ ID NO: selected from 4, 8, 12 and 27. In some embodiments, the cell further comprises an armoring genomic modification. In some embodiments, the armoring genomic modification comprises inactivation of one or more or two or more of an immune checkpoint gene or a regulatory gene selected from the group consisting of PDCD1, CTLA-4, LAG3, Tim3, BTLA, BY55, TIGIT, B7H5, LAIR1, SIGLEC10, B2M, CISH, CBLB and 2B4. In some embodiments, the armoring genomic modification comprises inactivation of CISH. In some embodiments, the armoring genomic modification comprises inactivation of CBLB. In some embodiments, the armoring genomic modification comprises inactivation of PDCD1. In some embodiments, the armoring genomic modification comprises inactivation of Tim3. In some embodiments, the armoring genomic modification comprises inactivation of LAG3. In some embodiments, the armoring genomic modification comprises inactivation of TIGIT. In some embodiments, the armoring genomic modification comprises inactivation of B2M. In some embodiments, the armoring genomic modification further comprises insertion of an HLA-E-B2M fusion construct into the B2M gene. In some embodiments, the cell is engineered to express a cytokine. In some embodiments, the cytokine is membrane-bound. In some embodiments, the cytokine is expressed as a cytokine-receptor fusion protein. In some embodiments, the cytokine is selected from IL-15 and IL-21. In some embodiments, the cytokine is selected from mbIL-15 and mbIL-21.

In one embodiment, the invention is a method of making the immune cell described herein, the method comprising introducing into a cell a nucleic acid comprising a sequence selected from SEQ ID NOs: 14, 16, 18, 36, and 38. In some embodiments, the cell is selected from a T cell, an NK cell, and an induced pluripotent stem cell (iPSC). In some embodiments, the cell is an iPSC, and the method further comprises differentiating the iPSC into an immune cell. In some embodiments, the introducing step comprises introducing into the cell a sequence-dependent endonuclease. In some embodiments, the introducing step comprises introducing into the cell a CRISPR system comprising a nucleic acid-guided endonuclease and nucleic acid-targeting nucleic acid (NATNA) guides. In some embodiments, the nucleic acid-guided endonuclease is selected from Cas9, Casl2a and CASCADE. In some embodiments, the endonuclease comprises a catalytically inactive CRISPR endonuclease conjugated to the cleavage domain of the restriction endonuclease Fok I. In some embodiments, the endonuclease is selected from the group consisting of a zinc finger nuclease (ZFN), a ZFN-Fok I fusion, a transcription activator-like effector nuclease (TALEN), and a TALEN-Fok I fusion. In some embodiments, the endonuclease cleaves the genome of the cell at a locus selected from the group consisting of TRAC, CBLB, PDCD1, CTLA-4, LAG3, Tim3, BTLA, BY55, TIGIT, B7H5, LAIR1, SIGLEC10, 2B4, and B2M In some embodiments, the nucleic acid comprising a sequence selected from SEQ ID NOs: 14, 16, 18, 36 and 38 comprises a vector. In some embodiments, the vector is a viral vector derived from a virus selected from the group consisting of an adenovirus type 2 and an adenovirus type 5, a retrovirus, a lentivirus, an adeno-associated virus (AAV), a simian virus 40 (SV-40), vaccinia virus, Sendai virus, Epstein-Barr virus (EBV), and herpes simplex virus (HSV).

In one embodiment, the invention is a composition comprising the immune cells described herein and a pharmaceutically acceptable excipient. In some embodiments, the immune cells are CAR-T cells in the amount of between 1×10⁶ and 2×10⁸ cells. In some embodiments, the immune cells are CAR-NK cells in the amount of between 1×10⁷ and 2×10⁹ cells. In some embodiments, the immune cells are a mixture of CAR-T cells and CAR-NK cells present at a ratio of approximately 1:10 CAR-T to CAR-NK. In some embodiments, the pharmaceutically acceptable excipient comprises one or more of carbohydrates, inorganic salts, antimicrobial agents, antioxidants, surfactants, buffers, acids, bases, water, alcohols, polyols, glycerin, vegetable oils, phospholipids, surfactants, sugars, derivatized sugars, alditols, mannitol, xylitol, maltitol, lactitol, xylitol, sorbitol, pyranosyl sorbitol, myoinositol, aldonic acid, esterified sugars, sugar polymers, monosaccharides, fructose, maltose, galactose, glucose, D-mannose, sorbose, disaccharides, lactose, sucrose, trehalose, cellobiose, polysaccharides, raffinose, melezitose, maltodextrins, dextrans, starches, citric acid, sodium chloride, potassium chloride, sodium sulfate, potassium nitrate, and sodium phosphate. In some embodiments, the antimicrobial agent comprises one or more of benzalkonium chloride, benzethonium chloride, benzyl alcohol, cetylpyridinium chloride, chlorobutanol, phenol, phenylethyl alcohol, phenylmercuric nitrate, and thimerosal. In some embodiments, the composition further comprises an antioxidant selected from ascorbyl palmitate, butylated hydroxyanisole, butylated hydroxytoluene, hypophosphorous acid, monothioglycerol, propyl gallate, sodium bisulfite, sodium formaldehyde sulfoxylate, and sodium metabisulfite. In some embodiments, the composition further comprises a surfactant selected from polysorbates, sorbitan esters, lecithin, phosphatidylcholines, phosphatidylethanolamines, fatty acids, fatty acid esters and cholesterol. In some embodiments, composition further comprises a freezing agent selected from 3% to 12% dimethylsulfoxide (DMSO) and 1% to 5% human albumin. In some embodiments, composition further comprises a preservative selected from one or more of methylparaben, propylparaben, sodium benzoate, benzalkonium chloride, antioxidants, chelating agents, parabens, chlorobutanol, phenol, and sorbic acid.

In one embodiment, the invention is a method of inhibiting the growth of a tumor in a patient comprising administering to a patient having the tumor the composition described herein. In some embodiments, the tumor is a solid tumor selected from ovarian cancer, triple negative breast cancer, colorectal cancer, non-small cell lung cancer, lung adenocarcinoma, pancreatic cancer, gastric cancer, melanoma, and endometrial carcinoma, or a hematological tumor selected from MCL, CLL, SLL, B-ALL, B-NHL, and AML. In some embodiments, the administering is selected from the group consisting of systemic delivery, parenteral delivery, intramuscular delivery, intravenous delivery, subcutaneous delivery, and intradermal delivery. In some embodiments, the composition further comprises a delivery-timing component that enables time-release, delayed release, or sustained release of the composition. In some embodiments, the delivery-timing component is selected from monostearate, gelatin, a semipermeable matrix, and a solid hydrophobic polymer. In some embodiments, the method further comprises administering a cytokine to the patient. In some embodiments, the cytokine is selected from IL-2, IL-12, IL-15, IL-18 and IL-21. In some embodiments, the method further comprises, prior to administering to the patient, applying to the immune cells a quality control measure comprising assessing one or more properties selected from presence of the anti-ROR1 CAR in the cellular genome, surface expression of the anti-ROR1 CAR, ROR1-dependent lysis of ROR1-expressing target cells, proliferation in the presence of ROR1-expressing target cells, cytokine or chemokine secretion in the presence of ROR1-expressing target cells, reducing tumor burden in experimental animals harboring ROR1-expressing tumors, and persistence in circulation of experimental animals harboring ROR1-expressing tumors upon administration of the immune cells to the animals. In some embodiments, the presence of the anti-ROR1 CAR in the cellular genome is assessed by a method selected from nucleic acid hybridization, nucleic acid sequencing, polymerase chain reaction (PCR), quantitative PCR (qPCR), real-time PCR (rtPCR) and droplet digital PCR (ddPCR). In some embodiments, the surface expression of the anti-ROR1 CAR is assessed by flow cytometry, fluorescence-activated cell sorting (FACS), microfluidics-based screening, ELISA, or Western blot. In some embodiments, the surface expression of the anti-ROR1 CAR is assessed by flow cytometry. In some embodiments, the immune cell population with the highest surface expression of the anti-ROR1 CAR is selected for administration to the patient. In some embodiments, the ROR1-dependent lysis of ROR1-harboring target cells is assessed by co-culturing the immune cells comprising the CAR with ROR1-expressing target cells at an effector:target ratio between about 0.1 and about 10 and assessing target cell lysis. In some embodiments, the immune cell population with the highest rate of lysis of ROR1-harboring target cells is selected for administration to the patient. In some embodiments, the ROR1-dependent proliferation is assessed by co-culturing the immune cells with ROR1-expressing target cells and assessing the proliferation of the immune cells. In some embodiments, the immune cell population with the highest rate of proliferation in the presence of ROR1-expressing target cells is selected for administration to the patient. In some embodiments, the cytokine or chemokine is selected from IFN-γ, TNF-α, GM-CSF, IL-10, IL-5, and IL-13, MIP-1α, MIP-1β, IL-8, and RANTES. In some embodiments, the cytokine secretion is assessed by co-culturing the immune cells with ROR1-expressing target cells and measuring the amount of cytokines in the co-culture supernatant. In some embodiments, the immune cell population with the highest cytokine secretion is selected for administration to the patient. In some embodiments, the reducing tumor burden in experimental animals harboring ROR1-expressing tumors is measured as change bioluminescence of the bioluminescent tumor cells in a time period after the animals have been injected with the immune cells. In some embodiments, the change in bioluminescence is expressed as area under the curve (AUC). In some embodiments, the immune cell population with the smallest AUC is selected for administration to the patient. In some embodiments, the persistence in circulation of experimental animals harboring ROR1-expressing tumors upon administration of the immune cells to the animals is measured by counting human CD56-expressing cells in the circulation of the animals. In some embodiments, the immune cell population with the highest counts of human CD56-expressing cells in the circulation of the animals is selected for administration to the patient. In some embodiments, the immune cell comprises an armoring genomic modification. In some embodiments, the genomic modification comprises inactivation of one or more of CISH, CBLB, B2M, PDCD1, Tim3, LAG3, and TIGIT. In some embodiments, the immune cell is engineered to express a cytokine. In some embodiments, the cytokine is selected from mbIL-15 and mbIL-21. In some embodiments, the method further comprises, prior to administering to the patient, applying to the immune cells a quality control measure comprising assessing one or more properties selected from surface expression of the cytokine, ROR1-dependent lysis of ROR1-expressing target cells, and reducing tumor burden in experimental animals harboring ROR1-expressing tumors. In some embodiments, the immune cell population with the highest surface expression of the cytokine is selected for administration to the patient. In some embodiments, the immune cell population with the highest ROR1-dependent lysis of ROR1-expressing target cells is selected for administration to the patient. In some embodiments, the immune cell population with the highest rate of reducing tumor burden in experimental animals harboring ROR1-expressing tumors is selected for administration to the patient. In some embodiments, the composition comprises CAR-T cells in the amount of between 1×10⁷ and 2×10⁸ cells, or CAR-NK cells in the amount of between 1×10⁸ and 2×10⁹ cells, or a mixture of CAR-T cells and CAR-NK cells present at a ratio of approximately 1:10 CAR-T to CAR-NK.

In one embodiment, the invention is a method of manufacturing anti-ROR1 immune cells, the method comprising introducing into a cell population a nucleic acid encoding a chimeric antigen receptor (CAR) comprising: an anti-ROR1 scFv; a transmembrane domain; a hinge domain; and a cytoplasmic domain, wherein the nucleic acid comprises a sequence selected from SEQ ID NOs: 4, 8, 12, and 27. In some embodiments, the cell population is selected from T cells, natural killer (NK) cells and cells capable of differentiating into NK cells. In some embodiments, the cells capable of differentiating into NK are selected from induced pluripotent stem cells (iPSC), hematopoietic progenitor cells (HPC) and lymphoid progenitor cells. In some embodiments, the introducing is into iPSCs, thereby forming CAR-iPSCs. In some embodiments, the method further comprises inducing differentiation of the CAR-iPSCs into induced CAR-NKs (CAR-iNKs). In some embodiments, the inducing differentiation comprises: contacting the CAR-iPSCs with one or more cytokines selected from BMP4, VEGF, SCF, IL3, IL6, and TPO to produce hematopoietic progenitor cells (HPC); enriching the HPCs by selecting CD34⁻ cells; contacting the HPCs with one or more cytokines selected from IL3, IL15, IL7, SCT, FLT3L in the presence feeder cells to produce induced natural killer cells (iNKs). In some embodiments, the method further comprises expanding the iNKs by a method comprising culturing the iNKs in the presence of feeder cells and cytokines. In some embodiments, the feeder cells secrete cytokines or express cytokines of the cell membrane. In some embodiments, the feeder cells express 4-1BB ligand (4-1BBL) and membrane-bound IL21 (mbIL21). In some embodiments, the nucleic acid is introduced into the precursor cell population via chemical or electrochemical means. In some embodiments, the nucleic acid is introduced into the precursor cell population via a vector selected from a plasmid vector and a viral vector. In some embodiments, the viral vector is derived from a virus selected from the group consisting of an adenovirus type 2 and an adenovirus type 5, a retrovirus, a lentivirus, an adeno-associated virus (AAV), a simian virus 40 (SV-40), vaccinia virus, Sendai virus, Epstein-Barr virus (EBV), and herpes simplex virus (HSV). In some embodiments, the introducing step comprises introducing into the cell a CRISPR system comprising a nucleic acid-guided endonuclease and nucleic acid-targeting nucleic acid (NATNA) guides. In some embodiments, the nucleic acid-guided endonuclease is selected from Cas9, Casl2a and CASCADE. In some embodiments, the endonuclease comprises a catalytically inactive CRISPR endonuclease conjugated to the cleavage domain of the restriction endonuclease Fok I. In some embodiments, the endonuclease is selected from the group consisting of a zinc finger nuclease (ZFN), a ZFN-Fok I fusion, a transcription activator-like effector nuclease (TALEN), and a TALEN-Fok I fusion.

### BRIEF DESCRIPTION OF THE DRAWINGS

FIG. 1 is a diagram of anti-ROR1 chimeric antigen receptors (CARs).

FIG. 2 is a workflow of generating induced ant-ROR1 CAR NK cells (iNK cells) from induced pluripotent stem cells (iPSCs).

FIG. 3 shows CAR expression in cells at various stages of the iNK pathway (FIG. 2) transformed with the anti-ROR1 CAR constructs shown in FIG. 1.

FIG. 4 shows ROR1 expression in cell lines SKOV3 and JeKo-1.

FIG. 5 shows SKOV3 tumor cell lysis (proportion of viable tumor cells) by the anti-ROR1 CAR iNK cells.

FIG. 6 shows JeKo1 tumor cell lysis (specific lysis of tumor cells) by the anti-ROR1 CAR iNK cells.

FIG. 7 shows repeated JeKo1 tumor cell lysis by the anti-ROR1 CAR iNK cells in a serial rechallenge assay.

FIG. 8 shows tumor burden in NGS mice after administration of the anti-ROR1 CAR iNK cells (shown as area under the curve, AUC).

FIG. 9 shows tumor burden in IL15 transgenic mice after administration of the anti-ROR1 CAR iNK cells (AUC).

FIG. 10 shows persistence of the human iNK cells in mouse circulation determined by quantifying human CD56⁺ cells.

FIG. 11 shows persistence of anti-ROR1 CAR expression in the iNK cells (hCD45⁺ CD56⁺ cells).

FIG. 12 shows cytokine secretion by CBLB-deficient iNK cells.

FIG. 13 shows assessment of the phenotype of the CBLB-deficient iNK cells.

FIG. 14 shows in vitro cytotoxicity of the CBLB-deficient iNK cells against SKOV3 tumor cells.

FIG. 15 shows degranulation of the CBLB-deficient iNK cells in cocultures with SKOV3 tumor cells.

FIG. 16 shows in vivo antitumor activity of the CBLB-deficient iNK cells in a mouse xenograft model.

FIG. 17 shows in vitro cytotoxicity of the CBLB-deficient iNK cells and the CISH-deficient iNK cells against SKOV3 tumor cells.

FIG. 18 shows effector molecule secretion by the CBLB-deficient iNK cells and the CISH-deficient iNK cells in cocultures with SKOV3 tumor cells (IFNγ and TNFα).

FIG. 19 shows effector molecule secretion (perforin and granzyme B) by the CBLB-deficient iNK cells and the CISH-deficient iNK cells in co-cultures with SKOV3 tumor cells.

FIG. 20 shows in vivo antitumor activity of the CBLB-deficient iNK cells and the CISH-deficient iNK cells in a mouse xenograft model (measured as AUC of tumor bioluminescence).

FIG. 21 shows in vivo antitumor activity of the CBLB-deficient iNK cells and the CISH-deficient iNK cells in a mouse xenograft model (measured as probability of survival of tumor-engrafted animals).

FIG. 22 shows cytotoxicity and survival in co-cultures with T cells of iNK protected against immune attack by a B2M-HLA-E fusion construct.

FIG. 23 shows the proportion of CD8+ cells in the CD56+ cell populations of T cells, iNK cells and cocultures.

FIG. 24 is a diagram of an expression construct for expressing a IL-15 cytokine-receptor fusion.

FIG. 25 shows cytotoxic properties of iNKs engineered to constitutively express the cytokine-receptor fusion.

FIG. 26 is a diagram of the original and optimized anti-ROR1 CAR expression constructs.

FIG. 27A, FIG. 27B, and FIG. 27C show specific target cell lysis (in vitro cytotoxicity) by anti-ROR1 CAR-T cells.

FIG. 28A and FIG. 28B show cytokine secretion by anti-ROR1 CAR-T cells in co-cultures with ROR1-expressing target cells.

FIG. 29A and FIG. 29B show in vivo antitumor activity of anti-ROR1 CAR-T cells measured as changes in body weight and probability of survival (FIG. 29A) and bioluminescence intensity (FIG. 29B)

FIG. 30A and FIG. 30B show specific target cell lysis (in vitro cytotoxicity) by checkpoint-deficient anti-ROR1 CAR-T cells.

FIG. 31A, FIG. 31B, FIG. 31C, and FIG. 31D show cytokine secretion by checkpoint-deficient anti-ROR1 CAR-T cells in co-cultures with ROR1-expressing target cells.

FIG. 32 shows results of a serial rechallenge of checkpoint-deficient anti-ROR1 CAR-T cells with ROR1-expressing tumor cells.

FIG. 33 shows specific target cell lysis (in vitro cytotoxicity) by multiple checkpoint-deficient anti-ROR1 CAR-T cells.

FIG. 34 shows specific target cell lysis (in vitro cytotoxicity) by anti-ROR1 CAR-T cells pCB7432, pCB7437 and pCB7438 (Table 3) against ROR1-positive and ROR1-negative cell lines and B-CLL primary cells.

FIG. 35A and FIG. 35B show specific target cell lysis (in vitro cytotoxicity) expressed as AUC by anti-ROR1 CAR-T cells pCB7429, pCB7430, pCB7431, pCB7433, pCB7434 and pCB7436 (Table 3) against ROR1-positive and ROR1-negative cell lines and B-CLL primary cells.

FIG. 36A and FIG. 36B show cytokine secretion by anti-ROR1 CAR-T cells T cells pCB7429, pCB7430, pCB7431, pCB7433, pCB7434 and pCB7436 (Table 3) in co-cultures with ROR1-positive and ROR1-negative target cells.

FIG. 37A and FIG. 37B show cytokine secretion by anti-ROR1 CAR-T cells pCB7432, pCB7437 and pCB7438 (Table 3) in co-cultures with ROR1-positive and ROR1-negative target cells.

FIG. 38 shows interleukin 36 gamma (IL36γ) secretion by anti-ROR1 CAR-T cells pCB7306 (FIG. 26), or pCB7436, pCB7437, and pCB7438, (Table 3) in the presence of ROR1-positive JeKo-1 cells.

FIG. 39A and FIG. 39B show in vivo anti-tumor efficacy of anti-ROR1 CAR-T cells pCB7430, pCB7436 and pCB7438 (Table 3).

FIG. 40 shows a comparison between the anti-ROR1 CAR-T cells and the benchmark anti-ROR1 CAR-T cells in surface CAR expression and CAR-T cells counts during serial rechallenge in vitro with ROR1 expressing target tumor cells.

FIG. 41 shows a comparison between the anti-ROR1 CAR-T cells and the benchmark anti-ROR1 CAR-T cells in target cell lysis (expressed as area under the curve, AUC) during serial rechallenge in vitro with ROR1 expressing target tumor cells.

FIG. 42 shows a comparison between the anti-ROR1 CAR-T cells and the benchmark anti-ROR1 CAR-T cells in reducing tumor burden (expressed as tumor bioluminescence and AUC of the bioluminescence measurement) in animals engrafted with Jeko-1 ROR1 expressing tumors.

FIG. 43 shows a comparison between the anti-ROR1 CAR-T cells and the benchmark anti-ROR1 CAR-T cells in prolonging overall survival of animals engrafted with Jeko-1 ROR1 expressing tumors.

FIG. 44 shows reducing tumor burden (expressed as tumor bioluminescence) in animals engrafted with ROR1 expressing tumors by anti-ROR1 CAR-iNK cells lacking CBLB and B2M expression but expressing a B2M-HLA-E fusion and an IL15 receptor fusion.

FIG. 45 shows reducing tumor burden (expressed as AUC of tumor bioluminescence measurements) in animals engrafted with ROR1 expressing tumors by anti-ROR1 CAR-iNK cells lacking CBLB and B2M expression but expressing a B2M-HLA-E fusion and an IL15 receptor fusion.

FIG. 46 shows results of a serial rechallenge in vitro of anti-ROR1 CAR-iNK cells lacking CBLB and B2M expression but expressing a B2M-HLA-E fusion and an IL15 receptor fusion with SKOV3, a tumor cell line with low ROR1 antigen density.

FIG. 47 shows results of a serial rechallenge in vitro of anti-ROR1 CAR-iNK cells lacking CBLB and B2M expression but expressing a B2M-HLA-E fusion and an IL15 receptor fusion with Hs746t, a tumor cell line with high ROR1 antigen density.

FIG. 48A and FIG. 48B show results of multiple rounds of treatment of animals engrafted with luciferase-expressing SKOV3 (a tumor cell line with low ROR1 antigen density) with anti-ROR1 iNK cells. FIG. 48A: bioluminescence; FIG. 48B: area under the curve (AUC) calculated from bioluminescence.

FIG. 49A and FIG. 49B show results of multiple rounds of treatment of animals engrafted with luciferase-expressing Hs746t (a tumor cell line with high ROR1 antigen density) with anti-ROR1 iNK cells. FIG. 49A: bioluminescence; FIG. 49B: area under the curve (AUC) calculated from bioluminescence.

### DETAILED DESCRIPTION OF THE INVENTION

### Definitions

The following definitions are provided to aid in understanding of the disclosure. Unless defined in this section, technical and scientific terms used in this disclosure have the meaning commonly understood by a person of ordinary skill in the art. See, e.g., Sambrook et al., Molecular Cloning, A Laboratory Manual, 4^{th} Ed. Cold Spring Harbor Lab. Press (2012).

The term “activation” refers to the state of a T cell that includes one or both of cell proliferation and cytokine secretion by the cell.

The term “antibody” refers to an immunoglobulin molecule which specifically binds to an antigen. The term also refers to antibody fragments including Fv, Fab and F(ab)₂, scFv and other forms described e.g., in Antibodies: A Laboratory Manual, 2^{nd} Ed. Greenfield, E., ed., Cold Spring Harbor Lab. Press, N.Y. (2013).

The term “co-stimulatory domain” refers to a part of a chimeric T cell receptor (CAR) which is a binding partner that specifically binds a co-stimulatory ligand, thereby mediating a co-stimulatory response of the T cell, proliferation, and cytokine secretion. Examples of co-stimulatory ligands include CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, and HVEM. Examples of co-stimulatory domains include CD27, CD28, 4-1BB, OX40, CD30, CD40, PD-1, ICOS, LFA-1, CD2, CD7, LIGHT, NKG2C, and B7-H3.

The term “therapeutic benefit” refers to an effect that improves the condition of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the tumor, or prevention of metastasis, or prolonging overall survival (OS) or progression free survival (PFS) of a subject with cancer.

The terms “pharmaceutically acceptable” and “pharmacologically acceptable” refer to molecular entities and compositions that do not produce an adverse, allergic, or other deleterious reaction in a patient. For example, the pharmaceutically and pharmacologically acceptable preparations should meet the standards set forth by the FDA Office of Biological Standards.

The term “pharmaceutically acceptable carrier” and “excipient” refer to aqueous solvents (e.g., water, aqueous solutions of alcohols, saline solutions, sodium chloride, Ringer's solution, etc.), non-aqueous solvents (e.g., propylene glycol, polyethylene glycol, vegetable oil, and injectable organic esters), as well as dispersion media, coatings, surfactants, gels, antioxidants, preservatives (e.g., antibacterial or antifungal agents, anti-oxidants, chelating agents, and inert gases), isotonic agents, absorption delaying agents, stabilizers, binders, disintegration agents, lubricants, sweetening agents, flavoring agents, and dyes. The concentration and pH of the various components in a pharmaceutical composition are adjusted according to well-known parameters for each component.

The term “domain” refers to one region in a polypeptide which is folded into a particular structure independently of other regions.

The term “effector function” refers to a specialized function of a differentiated cell, such as a NK cell.

The term “adoptive cell” refers to a cell that can be genetically modified for use in a cell therapy treatment. Examples of adoptive cells include T cells, macrophages, and natural killer (NK) cells.

The term “cell therapy” refers to the treatment of a disease or disorder that utilizes genetically modified cells. The term “adoptive cell therapy (ACT)” refers to a therapy that uses genetically modified adoptive cells. Examples of ACT include T cell therapies, CAR T cell therapies, natural killer (NK) cell therapies and CAR NK cell therapies.

The term “lymphocyte” refers to a leukocyte that is part of the vertebrate immune system. Lymphocytes include T cells such as CD4⁺ and/or CD8⁺ cytotoxic T cells, alpha/beta T cells, gamma/delta T cells, and regulatory T cells. Lymphocytes also include natural killer (NK) cells, natural killer T (NKT) cells, cytokine induced killer (CIK) cells, and antigen presenting cells (APCs), such as dendritic cells. Lymphocytes also include tumor infiltrating lymphocytes (TILs).

The terms “effective amount” and “therapeutically effective amount” of a composition such as a cell therapy composition, refer to a sufficient amount of the composition to provide the desired response in the patient to whom the composition is administered.

The terms “peptide,” “polypeptide,” and “protein” are interchangeable and refer to polymers of amino acids, including natural and synthetic (unnatural) amino acids, as well as amino acids not found in naturally occurring proteins, e.g., peptidomimetics, and D optical isomers. A polypeptide may be branched or linear and be interrupted by non-amino acid residues. The terms also encompass amino acid polymers that have been modified through acetylation, disulfide bond formation, glycosylation, lipidation, phosphorylation, cross-linking, or conjugation (e.g., with a label). The polypeptide need not include the full-length amino acid sequence of the reference molecule but can include only so much of the reference molecule as necessary in order for the polypeptide to retain its desired activity. For example, polypeptides comprising full-length proteins, fragments thereof, polypeptides with amino acid deletions, additions, and substitutions are encompassed by the terms “protein” and “polypeptide,” as long as the desired activity is retained. For example, polypeptides with 95%, 90%, 80%, or less of sequence identity with the reference polypeptide are included as long the desired activity is retained by the polypeptides.

The terms “CRISPR” (clustered regularly interspaced short palindromic repeats), “Cas” (CRISPR-associated protein) “CRISPR-Cas” and “CRISPR system” refer to the genome editing tool derived from prokaryotic organisms and comprising a nucleic acid guide molecule and a sequence-specific nucleic acid-guided endonuclease capable of cleaving a target nucleic acid strand at a site complementary to a sequence in the nucleic acid guide.

The term “NATNA” (nucleic acid targeting nucleic acid) refers to a nucleic acid guide molecule of the CRISPR system. NATNA may be comprised two nucleic acid targeting polynucleotides (“dual guide”) including a CRISPR RNA (crRNA) and transactivating CRISPR RNA (tracrRNA). NATNA may be comprised a single nucleic acid targeting polynucleotide (“single guide”) comprising crRNA and tracrRNA connected by a fusion region (linker). The crRNA may comprise a targeting region and an activating region. The tracrRNA may comprise a region capable of hybridizing to the activating region of the crRNA. The term “targeting region” refers to a region that is capable of hybridizing to a sequence in a target nucleic acid. The term “activating region” refers to a region that interacts with a polypeptide, e.g., a CRISPR nuclease.

Chimeric antigen receptor (CAR)-T cell therapies have demonstrated remarkable potential against hematologic malignancies (see e.g., U.S. Pat. No. 9,464,140). However, CAR-T cells have not shown such robust and reproducible antitumor activity against solid malignancies. Natural killer (NK) cells express the receptor NKG2D promoting their innate ability to detect and kill tumors that express stress ligands.

ROR1 (receptor tyrosine kinase-like orphan receptor 1, also known as NTRKR1, neurotrophic tyrosine kinase receptor-related 1) is an orphan receptor tyrosine-kinase-like antigen. ROR1 is 937 amino-acid protein, with amino acids 30-406 comprising the extracellular domain. ROR1 is a glycosylated type I membrane protein that belongs to the ROR subfamily of cell surface receptors. ROR1 binding to the specific ligand WNT5A activates the downstream Rac/Rho pathways responsible for cell motility and invasion.

ROR1 is expressed in a wide range of tumors including hematologic malignancies (chronic lymphocytic leukemia (CLL) and mantle cell lymphoma (MCL)), ovarian, lung, pancreatic, and triple negative breast cancer. ROR1 expression drives tumor cell growth, survival, and metastasis. Specifically, ROR1 has been shown to be expressed on ovarian cancer stem cells and promote migration, invasion, and cancer stem cell spheroid formation. Overexpression of ROR1 has been detected in both hematological cancers and solid tumors. Only low levels of ROR1 expression have been detected in normal human tissues such as adipose tissue, endocrine tissues, and the gastrointestinal tract making it a promising target for cellular immunotherapy against cancer.

Disclosed herein is an engineered T cells and T cell compositions, as wells as NK cells and NK cell compositions where the T cells and the NK cells are engineered to express a ROR1-specific CAR (anti-ROR1-CAR). The T cells and the NK cells of the invention have enhanced specificity and function against solid tumors and exhibit trafficking to ROR1⁺ tumors. The engineered T cells and NK cells can detect tumor cells via the anti-ROR1-CAR. Additionally, the engineered NK cells can detect tumor cells via its native NKG2D receptors.

Disclosed herein are methods and compositions for treatment of tumors with ROR1-targeting engineered cells including T cells and natural killer (NK) cells.

In some embodiments, the invention comprises adoptive cells and the use of adoptive cells in cellular immunotherapy. Adoptive cells of the instant invention include T cells, CAR-T cells, natural killer (NK) cells, induced natural killer (iNK) cells, CAR-NK cells, CAR-iNK cells and their precursors.

In some embodiments, the cells of the instant invention are allogeneic cells, i.e., cells isolated from a donor individual, such as a healthy human donor of either gender.

In some embodiments, the cells are isolated from a healthy donor using standard techniques. For example, lymphocytes can be isolated from blood, including peripheral blood and cord blood, or from lymphoid organs such as the thymus, bone marrow, lymph nodes, and mucosal-associated lymphoid tissues (MALT). Techniques for isolating lymphocytes from such tissues are well known in the art, see, e.g., Smith, J. W. (1997) Apheresis techniques and cellular immunomodulation, Ther. Apher. 1:203-206.

In some embodiments, isolated lymphocytes are characterized in terms of specificity, frequency and function. In some embodiments, the isolated lymphocyte population is enriched for specific subsets of cells, such as T cells or NK cells.

In some embodiments, the isolated lymphocyte population is enriched for specific subsets of T cells, such as CD4⁺, CD8⁺, CD25⁺, or CD62L⁺. See, e.g., Wang et al., Mol. Therapy—Oncolytics (2016) 3:16015. In some embodiments, the isolated lymphocyte population is enriched for CD56⁺ phenotype representing NK cells. In some embodiments, after isolation, the lymphocytes are activated in order to promote proliferation and differentiation into specialized lymphocytes. For example, T cells can be activated using soluble CD3/28 activators, or magnetic beads coated with anti-CD3/anti-CD28 monoclonal antibodies.

In some embodiments, NK cells are produced by differentiating stem cells such as embryonic stem cell (ESCc) and induced pluripotent stem cells (iPSCs). See e.g., T. Cheng (ed.), Hematopoietic Differentiation of Human Pluripotent Stem Cells, SpringerBriefs in Stem Cells, DOI 10.1007/978-94-017-7312-6_5, Hermanson, et al., Chapter 5, Human Pluripotent Stem Cells as a Renewable Source of Natural Killer Cells; Woll, et al., (2016) Human embryonic stem cells differentiate into a homogeneous population of natural killer cells with potent in vivo antitumor activity, Blood, 113(24) 6094; and Denman, et al., (2012) Membrane-Bound IL-21 Promotes Sustained Ex Vivo Proliferation of Human Natural Killer Cells, PloS One, 7(1):e30264.

The differentiation process typically comprises two and optionally, three steps: differentiating stem cells into hematopoietic progenitor cells (HPC), differentiating HPC into natural killer cells (NK), and optional expansion of NK cells. The products of differentiation (or expansion) may also be tested for the presence of NK phenotype such as NK-specific gene expression patterns, including expression of NK-specific cell surface markers, e.g., CD45 and CD56.

In some embodiments, the step of forming hematopoietic progenitor cells (HPC) comprises forming spheroids by allowing cells to aggregate (optionally assisted by low-speed centrifugation) and incubating the spheroids in presence of one or more cytokines selected from BMP4, VEGF, SCF, IL3, IL6, and TPO. In some embodiments, the combination of BMP4, VEGF, and SCF is used. In some embodiments, the combination of BMP4, VEGF, SCF, IL3, IL6, and TPO is used. In some embodiments, HPC are formed by incubation in the presence of feeder cells such as bone marrow stromal cells.

In some embodiments, the step of forming NK cells comprises incubating spheroids in the presence of one or more cytokines selected from IL-3, IL-15, IL-7, SCT, and FLT3L. In some embodiments, the combination of IL-3, IL-15, IL-7, SCT, and FLT3L is used. In some embodiments, feeder cells (stromal cells) are used. In some embodiments, fetal liver stromal cells are used as feeders. In some embodiments, the HPC fraction is enriched for the CD34⁺ cells prior to initiating the NK-differentiation step. In some embodiments, at the completion of the differentiation stage, the cell fraction is tested for surface expression of NK-specific markers such as CD45 and CD56.

In some embodiments, the step of NK cell expansion comprises incubating NK cells in the presence of cytokines and antigen presenting cells (APCs). In some embodiments, the APCs are engineered to express cytokines on the cell surface. In some embodiments, APCs overexpress 41BBL and membrane bound IL-21. In some embodiments, APCs overexpress membrane bound IL-15. In some embodiments, APCs overexpress cytokine-receptor fusions. In some embodiments, APCs overexpress an IL-15-IL-15R fusion. In some embodiments, APCs overexpress an IL-21-IL-21R fusion. In some embodiments, exogenous cytokines are also added. In some embodiments, IL-2 is added. In some embodiments, the final expanded iNK product was evaluated in in vitro and in vivo functional assays.

The present invention comprises allogeneic engineered immune cells including T cells and NK cells (including NK cells and induced NK (iNK) cells). In some embodiments, the cells described herein are genetically modified to express a chimeric antigen receptor (CAR). In some embodiments, the cells are CAR-T cells or CAR-NK cells. In some embodiments, the cells are CAR-iNK cells. In some embodiments, the invention comprises a combination of CAR-T cells and CAR-NK cells for administration to a patient. In some embodiments, the CAR-T cells and the CAR-NK cells are administered to a patient simultaneously as a single formulation. In some embodiments, the CAR-T cells and the CAR-NK cells are administered to a patient sequentially.

A typical chimeric antigen receptor (CAR) comprises an extracellular domain comprising an antigen binding region, a transmembrane domain, and one or more intracellular activation (co-stimulatory) domains. In some embodiments, the CAR also comprises a hinge domain. In some embodiments, the CAR also comprises a leader peptide directing the CAR to the cell membrane.

The CAR disclosed herein comprises an extracellular domain comprising an antigen binding region targeting ROR1. In some embodiments, the antigen binding region is derived from an antibody. In some embodiments, the antigen binding region is derived from a monoclonal antibody. In some embodiments, the antigen binding region comprises a mouse (murine) sequence. In some embodiments, the antigen binding region comprises a human sequence. In some embodiments, the antigen binding region comprises a humanized mouse (murine) sequence.

In some embodiments, the antigen binding region comprises a single-chain variable fragment (scFv). An scFv comprises a variable region of an antibody light chain (V_{L}) linked to a variable region of an antibody heavy chain (V_{H}). In some embodiments, the V_{L} is linked to the V_{H} via a peptide linker.

A peptide linker generally comprises from about 5 to about 40 amino acids. The linker can be a naturally occurring sequence or an engineered sequence. For example, in some embodiments, the linker is derived from a human protein, e.g., an immunoglobulin selected from IgG, IgA, I IgD, IgE, or IgM. In some embodiments, the linker comprises 5-40 amino acids from the CH1, CH2, or CH3 domain of an immunoglobulin heavy chain. In some embodiments, the linker is a glycine and serine rich linker having the sequence (GₓS_{y})ₙ. Additional linker examples and sequences are disclosed in the U.S. Pat. No. 5,525,491 Serine-rich peptide linkers, U.S. Pat. No. 5,482,858 Polypeptide linkers for production of biosynthetic proteins, and a publication WO2014087010 Improved polypeptides directed against IgE.

In some embodiments, the sequence of the scFv is further optimized for binding to the ROR1 antigen. In some embodiments, the optimization process comprises varying the linker between the heavy chain (V_{H}) and the light chain (V_{L}) of the scFv. It has been reported that the linker sequence can influence the antigen-scFv binding (Navabi, et al., (2021) Designing and optimization of a single-chain fragment variable (scFv) antibody against IL2Rα (CD25): An in silico and in vitro study, Iran J Basic Med Sci 24:1). Specifically, it has been shown that in silico design of the linker's amino acid sequence within the context of the known crystal structure of the scFv-antibody complex can yield linker sequences than impart better antigen affinity to the scFv. It has also been shown that chimeric antigen receptors (CARs) containing an scFv can be optimized for binding to the antigen simply by varying the length of the linker, where the linker comprises a variable number of repeats of the same sequence. Singh et al. (2021) Antigen-independent activation enhances the efficacy of 41BB co-stimulated CD22 CAR T cells, Nat. Med. 27(5):842. In some embodiments, the sequence is GₓS_{y} and the linker has the general formula (GₓS_{y})ₙ.

In some embodiments, the CAR comprises an scFv described in the international application Ser. No. PCT/US2023/067314 filed on May 22, 2023, Anti-ROR1 antibody and ROR1-targeting engineered cells.

In some embodiments, the CAR comprises the scFv selected from clones 857, 858 and 862 described in the PCT/US2023/067314. In some embodiments, the CAR comprises the scFv comprising a sequence selected from SEQ ID NO: 1, SEQ ID NO: 5, and SEQ ID NO: 9. In some embodiments, the CAR comprises the scFv consisting of a sequence selected from SEQ ID NO: 1, SEQ ID NO: 5, and SEQ ID NO: 9. In some embodiments, the scFv comprising a sequence of SEQ ID NO: 9 was further optimized to comprise SEQ ID NO: 27 containing the linker G₄S. In some embodiments, the optimized scFv consists of SEQ ID NO: 27.

In some embodiments, the scFv is encoded by a nucleic acid sequence comprising SEQ ID NO: 13, or SEQ ID NO: 15, or SEQ ID NO: 17, or SEQ ID NO: 28. In some embodiments, the scFv is encoded by a nucleic acid sequence consisting of SEQ ID NO: 13, or SEQ ID NO: 15, or SEQ ID NO: 17, or SEQ ID NO: 28.

In some embodiments, the antigen binding region comprises a heavy chain (V_{H}) comprising a sequence selected from SEQ ID NO: 2, SEQ ID NO: 6, and SEQ ID NO: 10. In some embodiments, the antigen binding region comprises a heavy chain (V_{H}) consisting of a sequence selected from SEQ ID NO: 2, SEQ ID NO: 6, and SEQ ID NO: 10.

In some embodiments, the antigen binding region comprises a light chain (V_{L}) comprising a sequence selected from SEQ ID NO: 3, SEQ ID NO: 7, and SEQ ID NO: 11. In some embodiments, the antigen binding region comprises a heavy chain (V_{H}) consisting of a sequence selected from SEQ ID NO: 3, SEQ ID NO: 7, and SEQ ID NO: 11.

Mouse antibodies and scFvs, humanized antibodies and scFvs derived therefrom, and human antibodies and scFvs comprise three complementarity determining regions (CDRs) in each of the heavy and light antibody chains (V_{H} and V_{L}).

In some embodiments, CDRs are identified using the crystal structure of the antigen-antibody complex. In some embodiments, CDRs are identified using in vitro methods such as phage display. In some embodiments, CDRs are identified using in silico methods, for example, IMGT (Lefranc et al., (2009) IMGT®, The international immunogenetics information system, Nucl. Acids Res. 37:D1006), and Kabat (Kabat et al., (1987) Sequences of Proteins of Immunological Interest, 4th ed., U.S. H.H.S., N.I.H.). In some embodiments, the CDRs are identified using the IMGT tool. In some embodiments, the CDRs are identified using the Kabat tool. In some embodiments, the minimal portions of the CDRs are identified as an overlap of the sequences located by the IMGT tool and the sequences located by the Kabat tool.

In some embodiments, the scFv included in the CAR of the instant invention comprises SEQ ID NO: 29 in the CDR1 of the V_{H}. In some embodiments, the scFv comprises SEQ ID NO: 30 in the CDR2 of the V_{H}. In some embodiments, the scFv comprises SEQ ID NO: 31 in the CDR3 of the V_{H}. In some embodiments, the scFv comprises SEQ ID NO: 32 in the CDR1 of the V_{L}. In some embodiments, the scFv comprises SEQ ID NO: 33 in the CDR2 of the V_{L}. In some embodiments, the scFv comprises SEQ ID NO: 34 in the CDR3 of the V_{L}.

In some the scFv included in the CAR of the instant invention comprises SEQ ID NO: 29 in the CDR1 of the V_{H}, and SEQ ID NO: 30 in the CDR2 of the V_{H}, and SEQ ID NO: 31 in the CDR3 of the V_{H} and further comprises SEQ ID NO: 32 in the CDR1 of the V_{L}, and SEQ ID NO: 33 in the CDR2 of the V_{L}, and SEQ ID NO: 34 in the CDR3 of the V_{L}.

In some embodiments, the CAR also comprises a hinge domain. In some embodiments, the hinge domain is derived from the CD8 protein. In some embodiments, the hinge domain comprises SEQ ID NO: 20. In some embodiments, the hinge domain consists of SEQ ID NO: 20. In some embodiments, the hinge domain is encoded by a nucleic acid comprising SEQ ID NO: 19. In some embodiments, the hinge domain is encoded by a nucleic acid consisting of SEQ ID NO: 19.

In some embodiments, the CAR comprises a signal peptide (a signal sequence) that enables trafficking of the CAR to the cell membrane. In some embodiments, the signal sequence comprises a CD28 signal sequence. In some embodiments, the signal sequence consists essentially of a CD28 signal sequence. In some embodiments, the signal sequence comprises a CD8 signal sequence. In some embodiments, the signal sequence consists essentially of a CD8 signal sequence.

In some embodiments, the transmembrane domain of the CAR is derived from a membrane-bound or transmembrane protein. In some embodiments, the transmembrane domain is derived from the same protein as the co-stimulatory domains described below. For example, the transmembrane domain of the CAR may be the transmembrane domain of a T cell receptor alpha-chain or beta-chain, a CD3-zeta chain, CD28, CD3-epsilon chain, CD45, CD2, CD4, CD5, CD8, CD9, CD16, CD22, CD27, CD33, CD37, CD64, CD80, CD86, CD134, CD137, ICOS, CD154, DNAM1, NKp44, NKp46, NKG2A, NKG2C, NKG2D, 2B4, TMIG2, TLR1, TLR2, TLR4, TLR5, TLR6, or GITR. In some embodiments, the transmembrane domain is the CD8 transmembrane domain. In some embodiments, the transmembrane domain comprises SEQ ID NO: 22. In some embodiments, the transmembrane domain consists of SEQ ID NO: 22. In some embodiments, the transmembrane domain is encoded by a nucleic acid comprising SEQ ID NO: 21. In some embodiments, the transmembrane domain is encoded by a nucleic acid consisting of SEQ ID NO: 21.

The cytoplasmic or intracellular signaling domain also referred to as the co-stimulatory domain of a CAR is responsible for activation of one or more effector functions of the immune cell expressing the CAR. In some embodiments, the co-stimulatory domain of the CAR comprises a part of or the entire sequence of the TCR zeta chain, CD3 zeta chain, CD3 epsilon chain, CD28, CD27, OX40/CD134, 4-1BB/CD137, ICOS/CD278, IL-2Rbeta/CD122, IL-2Ralpha/CD132, DAP10, DAP12, DNAM1, TLR1, TLR2, TLR3, TLR4, TLR5, TLR6, TLR7, TLR8, TLR9, MyD88, IL18R, CD40 or a combination thereof. In some embodiments, the cytoplasmic domain of the CAR comprises the CD3 zeta co-stimulatory domain. In some embodiments, the cytoplasmic domain of the CAR comprises the 4-1BB co-stimulatory domain. In some embodiments, the cytoplasmic domain of the CAR consists of the CD3 zeta and 4-1BB co-stimulatory domains.

In some embodiments, the CD3 zeta co-stimulatory domain comprises SEQ ID NO: 24. In some embodiments, the CD3 zeta co-stimulatory domain consists of SEQ ID NO: 24. In some embodiments, the CD3 zeta co-stimulatory domain is encoded by a nucleic acid comprising SEQ ID NO: 23. In some embodiments, the CD3 zeta co-stimulatory domain is encoded by a nucleic acid consisting of SEQ ID NO: 23.

In some embodiments, the 4-1BB co-stimulatory domain comprises SEQ ID NO: 26. In some embodiments, the 4-1BB co-stimulatory domain consists of SEQ ID NO: 26. In some embodiments, the 4-1BB co-stimulatory domain is encoded by a nucleic acid comprising SEQ ID NO: 25. In some embodiments, the 4-1BB co-stimulatory domain is encoded by a nucleic acid consisting of SEQ ID NO: 25.

In some embodiments, the cytoplasmic domain of the anti-ROR1 CAR comprises one or more additional elements that enhance intracellular signal transduction by the CAR. In some embodiments, the cytoplasmic domain comprises one or more protein association motifs that bind and recruit one or more intracellular signal transduction proteins. For example, U.S. Pat. No. 10,336,810 discloses increased cytotoxic activity of anti-CD19 CAR-T cells having CARs enhanced with JAK and STAT binding motifs in the cytoplasmic region.

In some embodiments, the CAR comprises two or more intracellular (or cytoplasmic) domains and two or more protein association motifs, wherein each protein association motif is in a separate intracellular domain of the CAR. In some embodiments, two or more protein association motifs are in the same intracellular domain of the CAR.

In some embodiments, the intracellular signal transduction protein is Signal Transducer and Activator of Transcription 3 or STAT3. In some embodiments, the STAT3 association motif is YXXQ (SEQ ID NO: 39). In some embodiments, the STAT3 association motif is YRHQ (SEQ ID NO: 40). The STAT3 association motif is naturally present in IL-6 and IL-10. In some embodiments, the STAT3 association motif is engineered into the CD3ζ intracellular signaling domain of the CAR.

In some embodiments, the intracellular signal transduction protein is Signal Transducer and Activator of Transcription 5 or STAT5. In some embodiments, the STAT5 association motif is YXXL (SEQ ID NO: 41). In some embodiments, the STAT5 association motif is YLSL (SEQ ID NO: 42). The STAT5 association motif is naturally present in IL-2R β chain. In some embodiments, the STAT5 association motif is engineered into the CD3ζ intracellular signaling domain of the CAR.

In some embodiments, the intracellular signal transduction protein is a Janus kinase such as JAK1. In some embodiments, the JAK association motif is LKCNTPDPS (SEQ ID NO: 43). In some embodiments, the JAK association motif is selected to the JAK association motifs present in IL2Rγ (IL2RG), Erythropoietin receptor (EpoR), thrombopoietin receptor (TpoR), granulocyte macrophage colony stimulating factor receptor (GM-CSFR), or growth hormone receptor (GHR). In some embodiments, the JAK association motif is engineered into the CD3ζ intracellular signaling domain of the CAR.

In some embodiments, the chimeric antigen receptor (CAR) comprises a sequence selected from SEQ ID NO.: 4, 8, 12, 35, and 37. In some embodiments, the CAR consists of a sequence selected from SEQ ID NO.: 4, 8, 12, 35, and 37. In some embodiments, the CAR is encoded by a nucleic acid comprising a sequence selected from SEQ ID NO: 14, 16, 18, 36, and 38. In some embodiments, the CAR is encoded by a nucleic acid consisting of a sequence selected from SEQ ID NO: 14, 16, 18, 36, and 38.

In some embodiments, the CAR is fully human or is humanized to reduce immunogenicity in human patients. In some embodiments, the CAR sequence is optimized for codon usage in human cells.

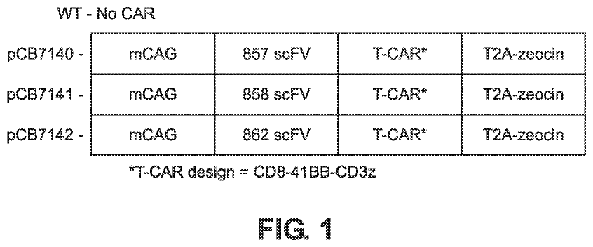

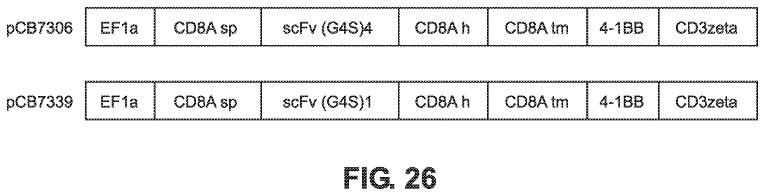

Examples of anti-ROR1 CAR constructs are described in Example 1 and FIG. 1 of the instant disclosure. Other examples of anti-ROR1 CAR constructs are described in Example 12 and FIG. 26 of the instant disclosure.

The nucleic acid encoding the CAR (e.g., nucleic acids comprising SEQ ID NO: 14, 16, 18, 36 or 38) may be introduced into a cell as a genomic DNA sequence or a cDNA sequence. The cDNA sequence comprises an open reading frame for the translation of the protein (e.g., CAR) and in some embodiments, the cDNA further comprises untranslated elements that improve for example, the stability or the rate of translation of the CAR mRNA.

In some embodiments, the CAR coding sequence is inserted into the cellular genome of a T cell or an NK cell at the endogenous T cell receptor alpha chain (TRAC) locus. In some embodiments, the TRAC locus is targeted by a CRISPR endonuclease (e.g., Cas9, Casl2a or CASCADE) and a guide polynucleotide. In some embodiments, the guide polynucleotide is a CRISPR hybrid DNA-RNA polynucleotide (chRDNA).

In some embodiments, the insertion of the CAR is reliant on the cell's endogenous homologous recombination systems. In such embodiments, the CAR coding sequence may be introduced into the cell via chemical or electrochemical means (such as lipid nanoparticle or electroporation). The CAR coding sequence can also be introduced into the cell using vectors such as lentiviral vectors as discussed in detail elsewhere in this disclosure.

In some embodiments, the cells used in the invention are engineered to express the CAR and further comprise a genome modification resulting in armoring of the cells against an attack by the immune system of a recipient of the allogeneic immune cells (immune cells derived from a donor). In some embodiments, the armoring modification comprises protection from recognition by the cytotoxic T cells of the host. Cytotoxic T cells recognize MHC Class I antigens. An MHC Class I molecule is a cell surface molecule comprised of beta-2 microglobulin (B2M) associated with heavy chains of HLA-I proteins (selected from HLA-A, HLA-B, HLA-C, HLA-E, HLA-F and HLA-G). The B2M/HLA-I complex on the surface of the allogeneic cell is recognized by cytotoxic CD8⁺ T cells and, if HLA-I is recognized as non-self, the allogeneic cell is killed by the T cells. In some embodiments, the cells of the invention comprise an armoring genomic modification comprising a disruption of the B2M gene and therefore, disruption of the MHC Class I cell surface-bound complex. This disruption eliminates the MHC Class I antigen recognition that normally stimulates a cytotoxic T cell attack.

In some embodiments, the armoring genome modification comprises disruption of recognition of the CAR-T cells or the CAR-NK cells by the natural killer (NK) cells of the host. NK cells recognize cells without MHC-I protein as “missing self” and kill such cells. NK cells are inhibited by HLA-I proteins, including HLA-E, a minimally polymorphic HLA-I protein. In some embodiments, the cells of the invention comprise a first armoring genomic modification comprising a disruption of the B2M gene and therefore, disruption of the MHC Class I cell surface-bound complex, disruption of the MHC Class I antigen recognition that stimulates a cytotoxic T cell attack, and further comprise a second armoring genomic modification comprising an insertion of an ILA-E gene fused to the beta-2-microglobulin (B2M) gene, and therefore, expression of the B2M-HLA-E construct designed to cloak the cells from an attack by NK cells. See, e.g., Gornalusse et al., (2017) HLA-E-expressing pluripotent stem cells escape allogeneic responses and lysis by NK cells, Nat. Biotechnol. (2017) 35:765-772.

The inventors have successfully achieved immune cloaking of iNK cells with a B2M-HLA-E fusion construct. One example of such immune cloaking is shown in Example 10.

In some embodiments, the armoring modification comprises transcriptionally silencing or disrupting one or more immune checkpoint or regulatory genes. In some embodiments, the checkpoint gene is selected from PD1 (encoded by the PDCD1 gene), CBLB, CISH (ISH), ADAM17, PRDM1, CTLA-4, LAG3, Tim3, BTLA, BY55, TIGIT, B7H5, LAIR1, SIGLEC10, B2M, and 2B4.

In some embodiments, the silenced or disrupted immune checkpoint gene is CBLB. CBLB protein is an immune checkpoint controlling activity of NK cells. Upon its phosphorylation, CBLB binds to activating downstream effectors of NK cells and downregulates them through proteasome-mediated degradation. CBLB negatively affects NK cell cytotoxicity, cytokine production and persistence, and suppressing CBLB activity in NK cells enhances the antitumor function of NK cells. See Lu et al., (2021) Cbl-b Is Upregulated and Plays a Negative Role in Activated Human NK Cells, J Immunol. 206 (4): 677-685; Lametschwandtner et al., (2015) Cbl-b silenced human NK cells respond stronger to cytokine stimulation, J ImmunoTher. of Cancer, 3 (Suppl. 2):P230; Guo, et al., (2021) CBLB ablation with CRISPR/Cas9 enhances cytotoxicity of human placental stem cell-derived NK cells for cancer immunotherapy. J ImmunoTher. of Cancer 9:e001975.

In some embodiments, the disruption of the CBLB gene by the methods described herein takes place in NK cells. In some embodiments, the NK cells are primary NK cells. In some embodiments, the NK cells are iNK cells (NK cells differentiated in vitro from iPSCs). In some embodiments, the disruption of the CBLB gene by the methods described herein takes place in iNK cell precursors such as iPSCs. In some embodiments, disruption of the CBLB gene in iPSCs does not negatively affect their ability to differentiate into iNK cells.

The inventors demonstrate herein that iNK cells lacking CBLB expression (CBLB KO) exhibit enhanced in vitro degranulation and cytotoxicity, as well as enhanced in vivo anti-tumor activity. The inventors also demonstrate that iPSCs with disrupted CBLB efficiently differentiate into iNKs, and the resulting iNK express the standard NK cell markers at levels similar to wild-type iNKs. (See Example 8 and Example 9).

In some embodiments, the silenced or disrupted regulatory gene is CISH. CISH or CIS (cytokine-induced SH2 protein) is a negative regulator of T cells and NK cells. Inhibition of CISH has been shown to enhance the potency of NK cells against certain cancers and infections (U.S. Pat. No. 11,104,375).

In some embodiments, disruption of the CISH gene by the methods described herein takes place in NK cells. In some embodiments, the NK cells are primary NK cells. In some embodiments, the NK cells are iNK cells (NK cells differentiated in vitro from iPSCs).

In some embodiments, disruption of the CISH gene by the methods described herein takes place in iNK cell precursors such as iPSC. In some embodiments, disruption of the CISH gene in iPSCs does not negatively affect their ability to differentiate into iNK cells.

The inventors demonstrate herein that iNK cells lacking CISH expression (CISH KO) exhibit enhanced in vitro cytotoxicity. (See Example 9.)

In some embodiments, the silenced or disrupted immune checkpoint gene is LAG3. Lymphocyte activation gene 3 (LAG3, also known as CD223) is an immune checkpoint receptor expressed on activated or exhausted T cells. LAG3 interacts with MHC class II molecules to inhibit T cell function and contributes to T cell exhaustion. Chronic lymphocytic leukemia (CLL) cells both express and secrete LAG3 which is thought to contribute to CLL tumor growth and escape from T cell attack. Shapiro et al., (2017) Lymphocyte activation gene 3: a novel therapeutic target in chronic lymphocytic leukemia, Haematologica, 102(5):874.

In some embodiments, disruption of the LAG3 gene by the methods described herein takes place in T cells. The inventors demonstrate herein that T cells lacking LAG3 expression exhibit enhanced in vitro cytotoxicity. (See Examples 17 and 18.)

In some embodiments, the silenced or disrupted immune checkpoint gene is Tim3. T cell immunoglobulin mucin 3 (Tim3) is a negative regulator of T cell function. Tim3 binds to its receptor Galectin-9 on the surface of T cells and inhibits the function of various types of T cells including CD4⁺ T cells, CD8⁺ T cells, Tregs and T helper cells. In hematologic malignancies including chronic lymphocytic leukemia (CLL), high levels of expression of Tim3 upregulate inhibitory Treg cells, inhibit T helper cell function and correlate with poor prognosis. Pang et al., (2021) Activated Galectin-9 Tim3 promotes Treg and suppresses Th1 effector function in chronic lymphocytic leukemia, FASEB J. 35:e21556.

In some embodiments, disruption of the Tim3 gene by the methods described herein takes place in T cells. The inventors demonstrate herein that T cells lacking Tim3 expression exhibit enhanced in vitro cytotoxicity (See Examples 17 and 18).

In some embodiments, the silenced or disrupted immune checkpoint gene is TIGIT. T cell immunoreceptor with immunoglobulin and ITIM domain (TIGIT) is expressed by activated CD8⁺ T and CD4⁺ T cells, natural killer (NK) cells, regulatory T cells (Tregs), and follicular T helper cells. High levels of TIGIT expression in CAR-T cells are associated with poor response to CAR-T cell therapy. Jackson et al., (2022) Sequential Single-Cell Transcriptional and Protein Marker Profiling Reveals TIGIT as a Marker of CD19 CAR-T Cell Dysfunction in Patients with Non-Hodgkin Lymphoma, Cancer Discov. 12(8):1886.

In some embodiments, disruption of the Tim3 gene by the methods described herein takes place in T cells. The inventors demonstrate herein that T cells lacking TIGIT expression exhibit enhanced in vitro cytotoxicity. (See Examples 17 and 18).

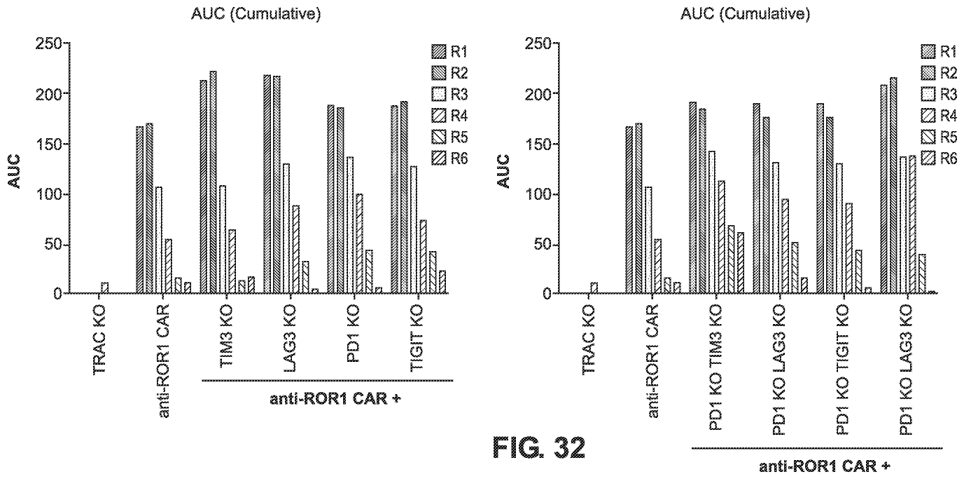

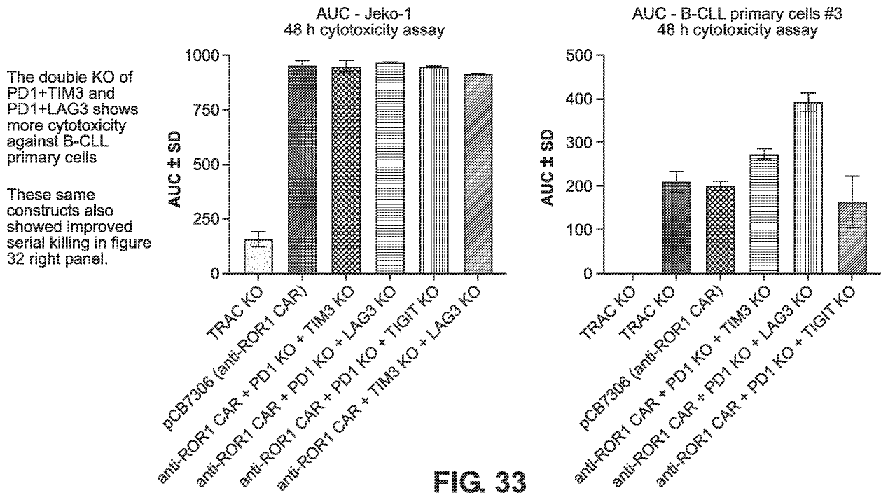

In some embodiments, multiple immune checkpoint genes are inactivated. In some embodiments, two or more immune checkpoint genes are inactivated in the same cell. In some embodiments, the multiple checkpoint genes include a combination of PD1 and Tim3, PD1 and LAG3, PD1 and TIGIT, and Tim3 and LAG3. The inventors have discovered that surprisingly, multiple checkpoint inactivation's in CAR-T cells have the most pronounced synergistic effect on duration of cytotoxic potential of the CAR-T cells (See FIGS. 32 and 33).

In some embodiments, the immune checkpoint gene or the regulatory gene is disrupted using an endonuclease that specifically cleaves nucleic acid strands within a target sequence of the gene to be disrupted. The strand cleavage by the sequence-specific endonuclease results in nucleic acid strand breaks that may be repaired by non-homologous end joining (NHEJ). NHEJ is an imperfect repair process that may result in direct re-ligation but more often, results in deletion, insertion, or substitution of one or more nucleotides in the target sequence. Such deletions, insertions, or substitutions of one or more nucleotides in the target sequence may result in missense or nonsense mutations in the protein coding sequence and eliminate production of any protein or cause production of a non-functional protein.

In some embodiments, the immune checkpoint gene is disrupted by contacting the cell with a sequence-specific endonuclease and triggering the NHEJ process within the cell resulting in gene mutation and elimination of protein expression of the immune checkpoint gene.

In some embodiments, the sequence-specific endonuclease is selected from a rare-cutting restriction enzyme, a TALEN, a Zinc-finger nuclease (ZFN) and a CRISPR endonuclease.

In some embodiments, the sequence-specific endonuclease is a CRISPR endonuclease selected from Cas9 and Casl2a. In some embodiments, the CRISPR endonuclease is part of a nucleoprotein complex comprising the CRISPR endonuclease and CRISPR guide RNA (nucleic acid targeting nucleic acid or NATNA). In some embodiments, the NATNA comprises one or more DNA nucleotides and is a CRISPR hybrid RNA-DNA or chRDNA. In some embodiments, the NATNA is selected from the embodiments described in U.S. Pat. No. 9,650,617. In some embodiments, the NATNA is selected from the embodiments described in the International Application Pub. No. WO2022086846 DNA-containing polynucleotides and guides for CRSIPR Type V systems, and methods of making and using the same.

In some embodiments, the invention comprises a method of producing the anti-ROR1 chimeric antigen receptor (CAR). In some embodiments, the nucleic acid encoding the CAR is introduced into a target cell where expression of the CAR is desired. In some embodiments, the introduced nucleic acid is selected from an expression vector containing the CAR-encoding sequence, an mRNA encoding the CAR, and a delivery vector containing the CAR-encoding donor sequence to be inserted into the cellular genome. In some embodiments, the target cells are contacted with the nucleic acid encoding the CAR in vitro, in vivo, or ex vivo.

In some embodiments, the vector used to deliver the CAR-encoding nucleic acid is a viral vector (e.g., a retroviral vector, adenoviral vector, adeno-associated viral vector, or lentiviral vector). In some embodiments, the vector is a lentiviral vector as di. Lentiviral vector packaging systems are available from multiple commercial vendors, e.g., ThermoFisher Scientific, LabCorp, and more. Such systems include a host cell where one or more plasmids encoding the sequence of interest (e.g., a CAR-coding sequence) are introduced together with the lentiviral vector coding genes. The transfected host cells then generate lentiviral vectors with the CAR sequence payload.

In some embodiments, the coding sequence of the CAR is introduced into the cells via a viral vector. Suitable vectors are non-replicating in the target cells. In some embodiments, the vector is selected from or designed based on SV40, EBV, HSV, or BPV. In some embodiments, the vector is a lentiviral vector or any other suitable viral vector capable of delivering an adequate-size payload. In some embodiments, to facilitate homologous recombination, the coding sequence is joined to homology arms located 5′ (upstream) and 3′ (downstream) of the insertion site in the desired insertion site in the genome. In some embodiments, the homology arms are about 500 bp long. See Eyquem J., et al. (2017) Targeting a CAR to the TRAC locus with CRISPR Cas9 enhances tumor rejection, Nature, 543:113-117. In some embodiments, the sequence coding for the CAR together with the homology arms are cloned into a viral vector plasmid. The plasmid is used to package the sequences into a virus.

The vector incorporates the protein expression sequences. In some embodiments, the expression sequences are codon-optimized for expression in mammalian cells. In some embodiments, the vector also incorporates regulatory sequences including transcriptional activator binding sequences, transcriptional repressor binding sequences, enhancers, introns, and the like. In some embodiments, the viral vector supplies a constitutive or an inducible promoter. In some embodiments, the promoter is selected from EF1α, PGK1, MND, Ubc, CAG, CaMKIIa, and β-Actin promoter. In some embodiments, the promoter is selected from the SV40 early and late promoters, the cytomegalovirus (CMV) immediate early promoter, and the Rous sarcoma virus long terminal repeat (RSV-LTR) promoter, mouse mammary tumor virus long terminal repeat (MMTV-LTR) promoter, the β-interferon promoter, the hsp70 promoter. In some embodiments, the promoter is the EF1α promoter. In some embodiments, the promoter is the MND promoter. In some embodiments, the promoter is the CAG promoter.

In some embodiments, the viral vector supplies a transcription terminator.

In some embodiments, the vector comprises a nucleic acid selected from SEQ ID NOs: 14, 16, 18, 36, and 38.

In some embodiments, the nucleic acid comprises more than one coding sequence and encodes a polycistronic transcript. In some embodiments, the first coding sequence in the polycistronic transcript encodes a CAR (e.g., SEQ ID NO) and the second coding sequence in the polycistronic transcript encodes a cytokine gene selected from IL-2, IL-12, IL-15, IL-18, IL-21, and IL36. In some embodiments, the cytokine is IL36. In some embodiments, the cytokine is IL36 with the IL36 signal peptide. In some embodiments, the cytokine is IL36 with the IL2 signal peptide. In some embodiments, the cytokine is IL36 with the IL2 mutated signal peptide described in Zhang, et al., (2005) Alteration in the IL-2 signal peptide affects secretion of proteins in vitro and in vivo, J. Gene Medicine, 7:354. The IL-2 mutated signal peptide contains modifications in two domains of the signal peptide: basic domain and hydrophobic domain. In some embodiments, the coding sequences in the polycistronic transcript are separated by the coding sequence for the P2A polypeptide to enable the separation of the nascent peptides during translation.

In some embodiments, the vector is a plasmid selected from a prokaryotic plasmid, a eukaryotic plasmid, and a shuttle plasmid.

In some embodiments, the CAR is expressed in a eukaryotic cell, such as a mammalian a human NK cell (or its precursor) and the vector is a plasmid comprising a eukaryotic promoter active in the desired cell type, a secretion signal, a polyadenylation signal, and a stop codon, and, optionally, one or more regulatory elements such as enhancer elements.

In some embodiments, the expression vector comprises one or more selection marker. In some embodiments, the selection markers are antibiotic resistance genes or other negative selection markers. In some embodiments, the selection markers comprise proteins whose mRNA is transcribed together with the fusion protein mRNA and the polycistronic transcript is cleaved prior to translation.

In some embodiments, the expression vector comprises polyadenylation signals. In some embodiments, the polyadenylation sites are SV-40 polyadenylation signals.

In some embodiments, the NK cells or precursors thereof are contacted with a viral vector so that the genetic material delivered by the vector is integrated into the genome of the target cell and then expressed in the cell or on the cell surface. In such embodiments, the transduced and transfected cells can be tested to confirm transgene expression on the cell surface using methods well known in the art such as fluorescence-activated cell sorting (FACS), microfluidics-based screening, ELISA, or Western blot. For example, the cells can be tested by staining or by flow cytometry with antibodies specific to a portion of the CAR or with a labeled antigen (e.g., ROR1 in the case of engineered anti-ROR1 CAR-T and CAR-NK cells).

The present invention involves manipulating nucleic acids, including genomic DNA and plasmid DNA that were isolated or extracted from a sample. Methods of nucleic acid extraction are well known in the art. See J. Sambrook et al., “Molecular Cloning: A Laboratory Manual,” 1989, 2nd Ed., Cold Spring Harbor Laboratory Press: New York, N.Y.). A variety of reagent and kits are commercially available for extracting nucleic acids (DNA or RNA) from biological samples, including products from BD Biosciences (San Jose, Cal.), Clontech (TaKaRa Bio.); Epicentre Technologies (Madison, Wisc.); Gentra Systems, (Minneapolis, Minn.); Qiagen (Valencia, Cal.); Ambion (Austin, Tex.); BioRad Laboratories (Hercules, Cal.); KAPA Biosystems (Roche Sequencing Solutions, Pleasanton, Cal.) and more.

In some embodiments, the invention involves intermediate purification or separation steps for nucleic acids, e.g., to remove unused reactants from the DNA. The purification or separation may be performed by a size selection method selected from gel electrophoresis, affinity chromatography and size exclusion chromatography. In some embodiments, size selection can be performed using Solid Phase Reversible Immobilization (SPRI) technology from Beckman Coulter (Brea, Cal.).

In some embodiments, exogenous protein-coding nucleic acid sequences (e.g., CAR-coding sequences) are introduced into a cell such as a T cell or an NK cell or an NK cell precursor such as an induced pluripotent stem cell (iPSC). In some embodiments, the “naked” nucleic acids are introduced into lymphocytes by electroporation as described e.g., in U.S. Pat. No. 6,410,319.

In some embodiments, the cell comprises the CRISPR system. In some embodiments, the CRISPR system comprises a nucleic acid-guided endonuclease and nucleic acid-targeting nucleic acid (NATNA) guides (e.g., a CRISPR guide RNAs selected from tracrRNA, crRNA or a single guide RNA incorporating the elements of the tracrRNA and crRNA in a single molecule). In some embodiments, the components of the CRISPR system are introduced into the cells (e.g., a T cell, an NK cell or an NK cell precursor) in the form of nucleic acids.

In some embodiments, the components of the CRISPR system are introduced into the cells (e.g., a T cell, an NK cell or an NK cell precursor) in the form of DNA coding for the nucleic acid-guided endonuclease and NATNA guides. In some embodiments, the gene coding for the nucleic acid-guided endonuclease (e.g., a CRISPR nuclease selected from Cas9 and Casl2a) is inserted into a plasmid capable of propagating in the target cell. In some embodiments, the gene coding for the NATNA guides is inserted into a plasmid capable of propagating in the target cell.

In some embodiments, the nucleic acid-guided endonuclease and NATNA guides are introduced into the target cells (e.g., a T cell, an NK cell or an NK cell precursor) in the form of RNA, e.g., the mRNA coding for the nucleic acid-guided endonuclease along with the NATNA guides.

In some embodiments, the nucleic acid-guided endonuclease and the NATNA guides are introduced into the target cells (e.g., a T cell, an NK cell or an NK cell precursor) as a preassembled nucleoprotein complex. In some embodiments, the nucleic acid-guided endonuclease and the NATNA guides are introduced into the target cells (e.g., T cells, NK cells or NK cell precursors) via any combination of different means, e.g., the endonuclease is introduced as the DNA via a plasmid containing the gene encoding the endonuclease while the guides are introduced in its final format as RNA (or RNA containing DNA nucleotides).

In some embodiments, the nucleic acids encoding the nucleic acid-guided endonuclease and NATNA guides are introduced into the cells via electroporation.

In some embodiments, the nucleic acids coding for the nucleic acid-guided endonuclease are introduced into cells in the form of mRNA as described e.g., in the U.S. Pat. No. 10,584,352 via electroporation or viral pseudo-transduction as described therein.

In some embodiments, one or more of the coding sequences described herein are introduced into the genome of the cell with the aid of a sequence-specific endonuclease. In some embodiments, the endonuclease is a nucleic acid-guided endonuclease encoded by the CRISPR locus. The CRISPR (Clustered Regularly Interspaced Short Palindromic Repeats) genomic locus is found in many prokaryotic genomes and provides resistance to invasion of foreign nucleic acids. Structure, nomenclature, and classification of CRISPR loci are reviewed in Makarova et al., Evolution and classification of the CRISPR-Cas systems. Nature Reviews Microbiology. 2011 June; 9(6): 467-477.

Briefly, a typical CRISPR locus includes a number of short repeats regularly interspaced with spacers. The CRISPR locus also includes coding sequences for CRISPR-associated (Cas) genes. A spacer-repeat sequence unit encodes a CRISPR RNA (crRNA). In vivo, mature crRNAs are processed from a polycistronic transcript referred to as pre-crRNA or pre-crRNA array. The repeats in the pre-crRNA array are recognized by Cas-encoded proteins that bind to and cleave the repeats liberating mature crRNAs. CRISPR systems perform cleavage of a target nucleic acid wherein Cas proteins and crRNA form CRISPR ribonucleoproteins (crRNP). The crRNA molecule guides the crRNP to the target nucleic acid (e.g., a foreign nucleic acid invading a bacterial cell) and the Cas nuclease proteins cleave the target nucleic acid.

Type I CRISPR systems include means for processing the pre-crRNA array that include a multi-protein complex called CASCADE (CRISPR-associated complex for antiviral defense) comprised of subunits CasA, B, C, D and E. The Cascade-crRNA complex recognizes the target nucleic acid through hybridization of the target nucleic acid with crRNA. The bound nucleoprotein complex recruits the Cas3 helicase/nuclease to facilitate cleavage of target nucleic acid.

Type II CRISPR systems include a trans-activating CRISPR RNA (tracrRNA). The tracrRNA hybridizes to a crRNA repeat in the pre-crRNA array and recruits endogenous RNaseIII to cleave the pre-crRNA array. The tracrRNA/crRNA complex can associate with a nuclease, e.g., Cas9. The crRNA-tracrRNA-Cas9 complex recognizes the target nucleic acid through hybridization of the target nucleic acid with crRNA. Hybridization of the crRNA to the target nucleic acid activates the Cas9 nuclease for target nucleic acid cleavage.

Type III CRISPR systems include the RAMP superfamily of endoribonucleases (e.g., Cas6) that cleave the pre-crRNA array with the help of one or more CRISPR polymerase-like proteins.

Type VI CRISPR systems comprise a different set of Cas-like genes, including Csf1, Csf2, Csf3, and Csf4 which are distant homologues of Cas genes in Type I-III CRISPR systems.

Type V CRISPR systems are classified into several different subtypes, including, e.g., V-A, V-B, V-C, V-D, V-E, V-F, V-G, V-H, V-I, V-J, V-K, and V-U. See, e.g., Makarova et al. (Nat. Rev. Microbiol., 2020, 18:67-83) and Pausch et al. (Science, 2020, 369(6501):333-337). The V-A subtype encodes the Casl2a protein (formerly known as Cpf1). Cas12a has a RuvC-like nuclease domain that is homologous to the respective domain of Cas9 but lacks the HNH nuclease domain that is present in Cas9 proteins. Type V systems can comprise a single crRNA sufficient for targeting of the Cas12 to a target site, or a crRNA-tracrRNA guide pair for targeting of the Cas12 to a target site.

CRISPR endonucleases require a nucleic acid targeting nucleic acid (NATNA) also known as guide RNAs. The endonuclease is capable of forming a ribonucleoprotein complex (RNP) with one or more guide RNAs. In some embodiments, the endonuclease is a Type II CRISPR endonuclease and NATNA comprises tracrRNA and crRNA.

In some embodiments, NATNA is selected from the embodiments described in U.S. Pat. No. 9,260,752. Briefly, a NATNA can comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a nucleic acid-targeting nucleic acid can comprise, a tracrRNA extension, a 3′ tracrRNA sequence, a minimum tracrRNA, a single guide connector, a minimum CRISPR repeat, a spacer, and a spacer extension in any order.

In some embodiments, the guide nucleic acid-targeting nucleic acid can comprise a single guide NATNA. The NATNA comprises a spacer sequence which can be engineered to hybridize to the target nucleic acid sequence. The NATNA further comprises a CRISPR repeat comprising a sequence that can hybridize to a tracrRNA sequence. Optionally, NATNA can have a spacer extension and a tracrRNA extension. These elements can include elements that can contribute to stability of NATNA. The CRISPR repeat and the tracrRNA sequence can interact, to form a base-paired, double-stranded structure. The structure can facilitate binding of the endonuclease to the NATNA.

In some embodiments, the single guide NATNA comprises a spacer sequence located 5′ of a first duplex which comprises a region of hybridization between a minimum CRISPR repeat and minimum tracrRNA sequence. The first duplex can be interrupted by a bulge. The bulge facilitates recruitment of the endonuclease to the NATNA. The bulge can be followed by a first stem comprising a linker connecting the minimum CRISPR repeat and the minimum tracrRNA sequence. The last paired nucleotide at the 3′ end of the first duplex can be connected to a second linker connecting the first duplex to a mid-tracrRNA. The mid-tracrRNA can comprise one or more additional hairpins.

In some embodiments, the NATNA can comprise a double guide nucleic acid structure. The double guide NATNA comprises a spacer extension, a spacer, a minimum CRISPR repeat, a minimum tracrRNA sequence, a 3′ tracrRNA sequence, and a tracrRNA extension. The double guide NATNA does not include the single guide connector. Instead, the minimum CRISPR repeat sequence comprises a 3′ CRISPR repeat sequence and the minimum tracrRNA sequence comprises a 5′ tracrRNA sequence and the double guide NATNAs can hybridize via the minimum CRISPR repeat and the minimum tracrRNA sequence.

In some embodiments, NATNA is an engineered guide RNA comprising one or more DNA residues (CRISPR hybrid RNA-DNA or chRDNA). In some embodiments, NATNA is selected from the embodiments described in U.S. Pat. No. 9,650,617. In some embodiments, NATNA is selected from the embodiments described in International Application Pub. No. WO2022086846 DNA-containing polynucleotides and guides for CRSIPR Type V systems, and methods of making and using the same. Briefly, some chRDNA for use with a Type II CRISPR system may be composed of two strands forming a secondary structure that includes an activating region composed of an upper duplex region, a lower duplex region, a bulge, a targeting region, a nexus, and one or more hairpins. A nucleotide sequence immediately downstream of a targeting region may comprise various proportions of DNA and RNA. Other chRDNA may be a single guide D(R)NA for use with a Type II CRISPR system comprising a targeting region, and an activating region composed of a lower duplex region, an upper duplex region, a fusion region, a bulge, a nexus, and one or more hairpins. A nucleotide sequence immediately downstream of a targeting region may comprise various proportions of DNA and RNA. For example, the targeting region may comprise DNA or a mixture of DNA and RNA, and an activating region may comprise RNA or a mixture of DNA and RNA.

In some embodiments, the endonuclease used to introduce one or more of the genetic modifications described herein (e.g., gene inactivation or insertion of the CAR-coding sequences) into the genome of a cell is a restriction endonuclease, e.g., a Type II restriction endonuclease.

In some embodiments, the endonuclease used to introduce one or more of the genetic modifications described herein is a catalytically inactive CRISPR endonuclease (e.g., catalytically inactive Cas9 or Casl2a) conjugated to the cleavage domain of the restriction endonuclease Fok I. (see e.g., Guilinger, J. P., et al., (2014). Fusion of catalytically inactive Cas9 to FokI nuclease improves the specificity of genome modification, Nature biotechnology, 32(6), 577-582.

In some embodiments the endonuclease the endonuclease used to introduce one or more of the genetic modifications described herein is a zinc finger nuclease (ZFN), or a ZFN-Fok I fusion. In such embodiments, the target sequence is about 22-52 bases long and comprises a pair of ZFN recognition sequences, each 9-18 nucleotides long, separated by a spacer, which is 4-18 nucleotides long. (See e.g., Kim Y. G., et al., (1996). Hybrid restriction enzymes: zinc finger fusions to Fok I cleavage domain, Proc Natl Acad Sci USA. 93(3): 1156-1160.

In some embodiments, the endonuclease the endonuclease used to introduce one or more of the genetic modifications described herein is a transcription activator-like effector nuclease (TALEN), or a TALEN-Fok I fusion. In such embodiments, the target sequence is about 48-85 nucleotides long and comprises a pair of TALEN recognition sequences, each 18-30 bases long, separated by a spacer, which is 12-25 bases long. (See e.g., Christian M. et al., (2010) Targeting DNA double-strand breaks with TAL effector nucleases, Genetics. 186 (2): 757-61.

In some embodiments, a quality control measure assessing one or more properties of the engineered anti-ROR1 CAR-T cells and CAR-NK cells is applied to the cells prior to administering the cells to a patient.

In some embodiments, the assessed property of the CAR-T cells and CAR-NK cells is the presence of the CAR nucleic acid in the cellular genome. The presence of the CAR in the cellular genome may be assessed by a method selected from nucleic acid hybridization, nucleic acid sequencing, and specific amplification including polymerase chain reaction (PCR), quantitative PCR (qPCR), real-time PCR (rtPCR) and droplet digital PCR (ddPCR). In some embodiments, the presence of the CAR in the cellular genome is assessed by ddPCR with amplification primers specific for one or both CAR insertion sites.

In some embodiments, the assessed property of the CAR-T cells and CAR-NK cells is surface expression of the CAR. The surface expression of the CAR may be assessed by fluorescence-activated cell sorting (FACS), microfluidics-based screening, ELISA, or Western blot. In some embodiments, the surface expression of the CAR is assessed by flow cytometry with an anti-Fab2 antibody or labeled purified antigen. In some embodiments, the surface expression of the CAR is assessed by flow cytometry with ROR1. In some embodiments, the CAR-T cell or CAR-NK cell population with the highest surface expression of the CAR is selected for administration to a patient.

In some embodiments, the fraction of cells in the cell population harboring the CAR in the genome, or the fraction of cells in the cell population expressing the CAR on the cell surface is used to determine the total number of cells of the cell population constituting a therapeutically effective dose.

In some embodiments, the properties of the CAR-T cells and CAR-NK cells are assessed in vitro and are selected from antigen-dependent lysis of antigen-expressing target cells (antigen-specific lysis); proliferation in the presence of antigen-expressing target cells (antigen-dependent proliferation); and cytokine secretion in the presence of antigen-expressing target cells, cell exhaustion, and the presence of a memory cell phenotype.

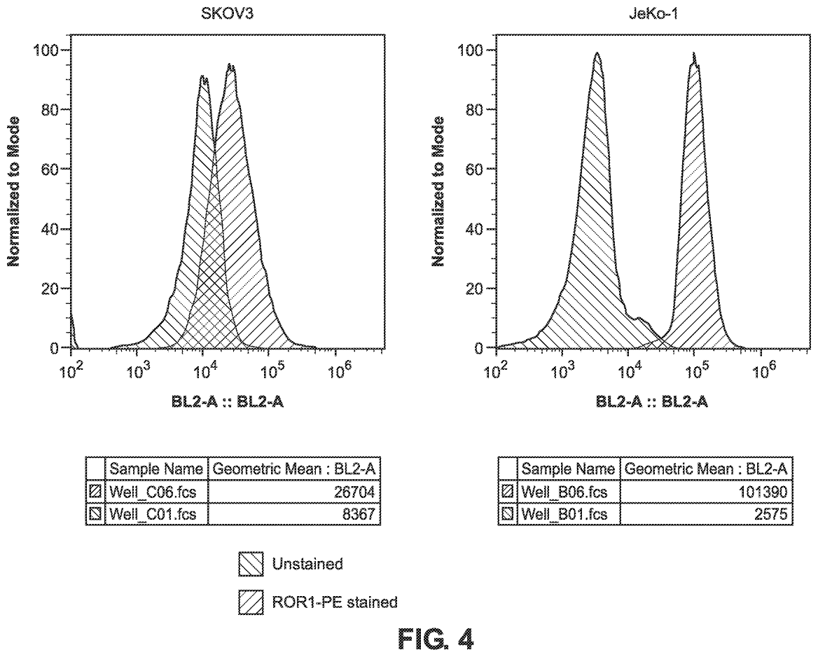

In some embodiments, the in vitro assessment of the CAR-T cells and CAR-NK cells utilizes target cells or target cell lines. In some embodiments, the target cells are tumor cells selected from primary tumor cells and established tumor cell lines. In some embodiments, the tumor cells are known to express the specific antigen for the CAR-T cell or the CAR-NK cell, i.e., the tumor cells express ROR1 recognized by the anti-ROR1 CAR-T cells and CAR-NK cells. In some embodiments, the tumor cells are from tumor cell lines SKOV3, REC1, Mino and JeKo-1. In some embodiments, the target cells are first assessed for ROR1 expression. An example of assessing ROR1 expression in target cells is presented as Example 3 and FIG. 4 of the instant disclosure.

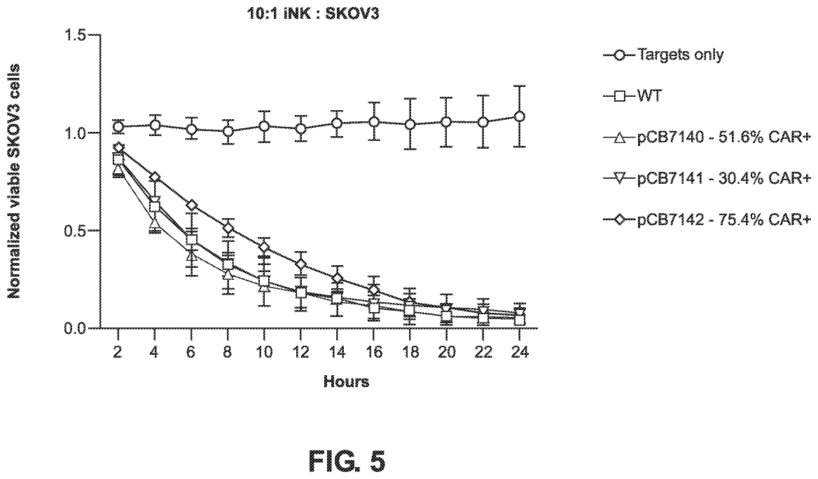

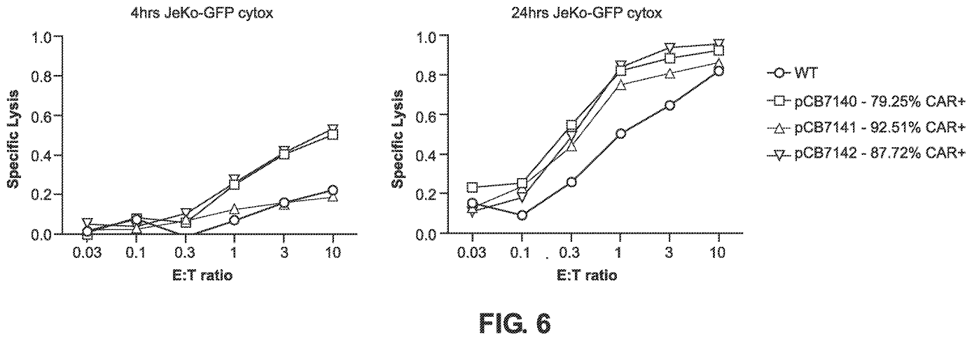

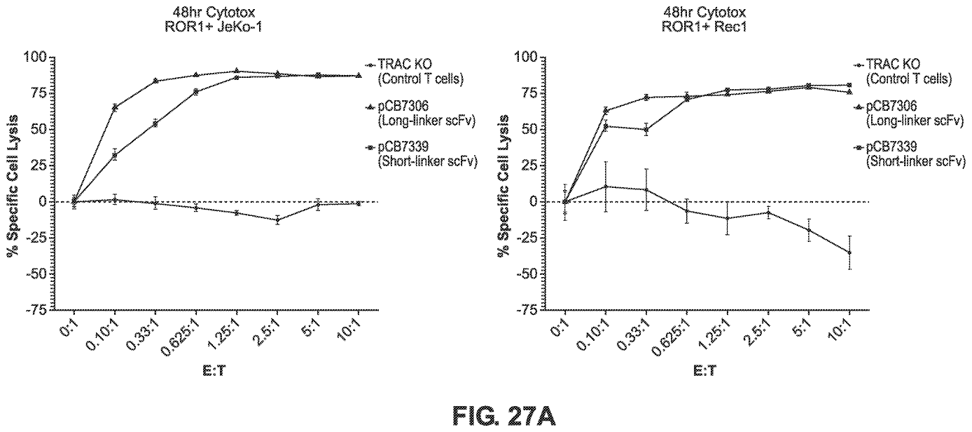

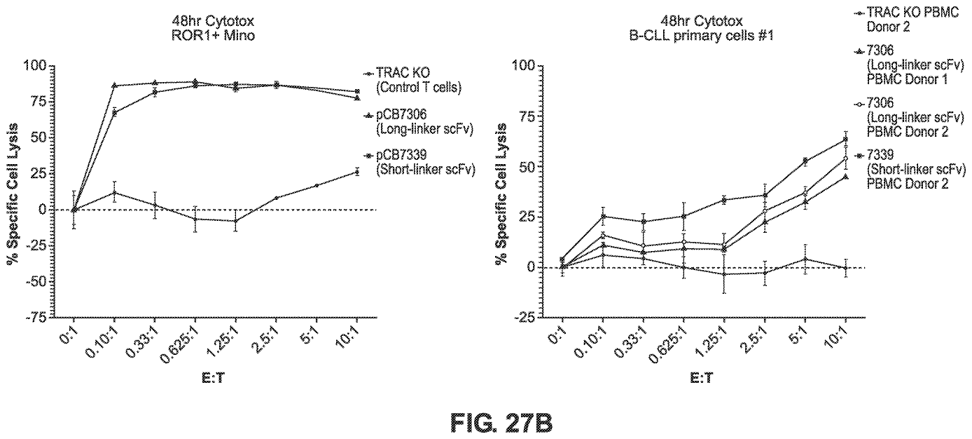

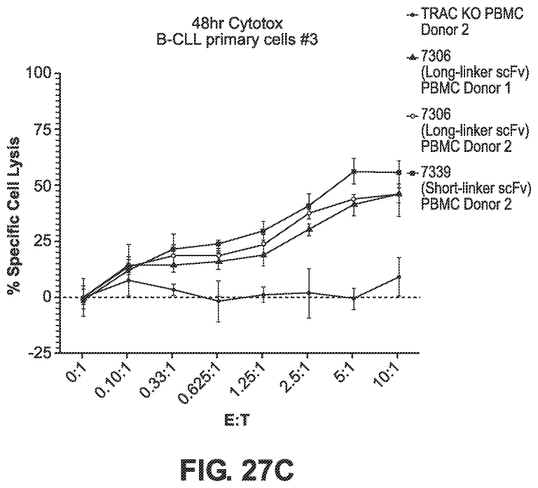

In some embodiments, the assessed property is antigen-dependent lysis of antigen-harboring target cells. The ROR1-dependent cell lysis may be assessed by co-culturing the population comprising engineered anti-ROR1 CAR-T cells or CAR-NK cells (effector cells or effectors) with ROR1-expressing target cells (targets). In some embodiments, the target cells are ROR1-expressing tumor cell lines. In some embodiments, the ROR1-expressing tumor cell lines are selected from JeKo1 (B cell lymphoma, BCL), REC1 (mantle cell lymphoma, MCL) and Mino (MCL). In some embodiments, the target cells are primary cells from patients suffering from tumors known to express ROR1. In some embodiments, the primary cells are from a lymphoma patient, e.g., a B cell lymphoma patient. The co-culture may be established at different effector:target ratios (E:T ratios). In some embodiments, the E:T ratios are in the range of about 0.1 and about 10. In some embodiments, two or more E:T ratios in the selected range are evaluated. In some embodiments, the ROR1-expressing target cells are labelled with a cell tracing dye (e.g., CellTrace™ Violet dye (CTV)) in advance and co-cultured with anti-ROR1 CAR-T or CAR-NK effector cells at a range of E:T ratios. At the analysis timepoints, a dead cell stain (e.g., 7-aminoactinomycin (7AAD) stain) is added to the cultures. The number of live target cells is then counted e.g., by flow cytometry as the number of trace stain/no dead stain (e.g., CTV⁺7AAD⁻) cells remaining. In some embodiments, the number of the live target cells is normalized against the number of live target cells in a control culture lacking the anti-ROR1 CAR-T or CAR-NK cells. An example of measuring in vitro target cell lysis by anti-ROR1 CAR-NK cells is presented as Example 3 and FIGS. 5 and 6 of the instant disclosure. An example of measuring in vitro target cell lysis by anti-ROR1 CAR-T cells is presented as Example 14 and FIGS. 27A-27C of the instant disclosure.

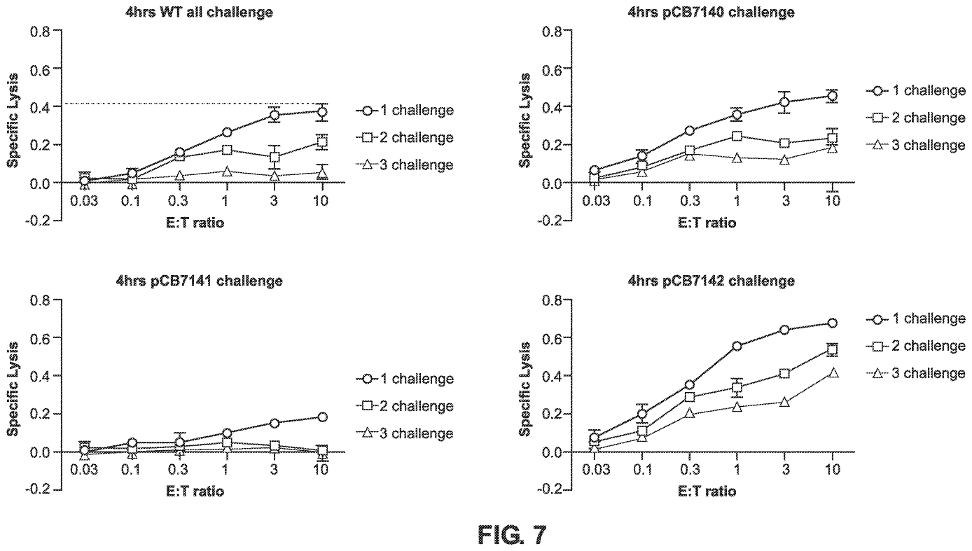

In some embodiments, the assessed property is repeated antigen-dependent lysis of antigen-harboring target cells in a serial rechallenge assay. The antigen-dependent cell lysis may be assessed by successive challenges of the same population of engineered anti-ROR1 CAR-T cells or CAR-NK cells with two or more fresh aliquots of target cells. An example of measuring repeated in vitro target cell lysis by anti-ROR1 CAR-NK cells is presented as Example 4 and FIG. 7 of the instant disclosure. An example of measuring repeated in vitro target cell lysis by anti-ROR1 CAR-T cells is presented as Example 20 and FIG. 32 of the instant disclosure.

In some embodiments, the CAR-T or CAR-NK cell population effecting the highest percentage of ROR1-expressing target cell lysis is selected for administration to a patient. In some embodiments, the CAR-T or CAR-NK cell population effecting a high percentage of ROR1-expressing target cell lysis but having low non-specific target cell lysis is selected for administration to a patient. In some embodiments, the CAR-T or CAR-NK cell population effecting the highest percentage of repeated ROR1-expressing target cell lysis is selected for administration to a patient.

In some embodiments, the assessed property is antigen-dependent proliferation of CAR-T or CAR-NK cells. Proliferation may be assessed by co-culturing a population comprising engineered anti-ROR1 CAR-T or CAR-NK cells (effectors, E) with ROR1-expressing target cells (targets, T). In some embodiments, the co-culture is at E:T ratio of about 1. In some embodiments, cell proliferation is detected by labeling CAR-T or CAR-NK cells with cell permeant stable fluorescent dyes (e.g., CellTrace™ Violet) and measuring dye dilution within the CAR-T or CAR-NK cell population.

In some embodiments, the CAR-T or CAR-NK cell population exhibiting the highest rate of proliferation in the presence of ROR1-expressing target cells is selected for administration to a patient.

In some embodiments, the assessed property is cytokine or chemokine secretion by the CAR-T or CAR-NK cells. In some embodiments, secretion of one or more cytokines or chemokines is assessed. The one or more cytokines are selected from IFN-γ, TNF-α, GM-CSF, IL-10, IL-5, and IL-13 and chemokines such as MIP-1α, MIP-1β, IL-8, and RANTES. Cytokine or chemokine secretion may be assessed by co-culturing a population comprising engineered anti-ROR1 CAR-T or CAR-NK cells (effectors, E) with a ROR1-expressing target cells (targets, T). In some embodiments, the co-culture is at E:T ratio of about 1. In some embodiments, the cytokines or chemokines in the co-culture supernatant can be detected or quantitatively detected by an antibody-based or antibody conjugate-based assay such as Western blotting or ELISA and similar secondary antibody-based methods with colorimetric or fluorescent detection methods.

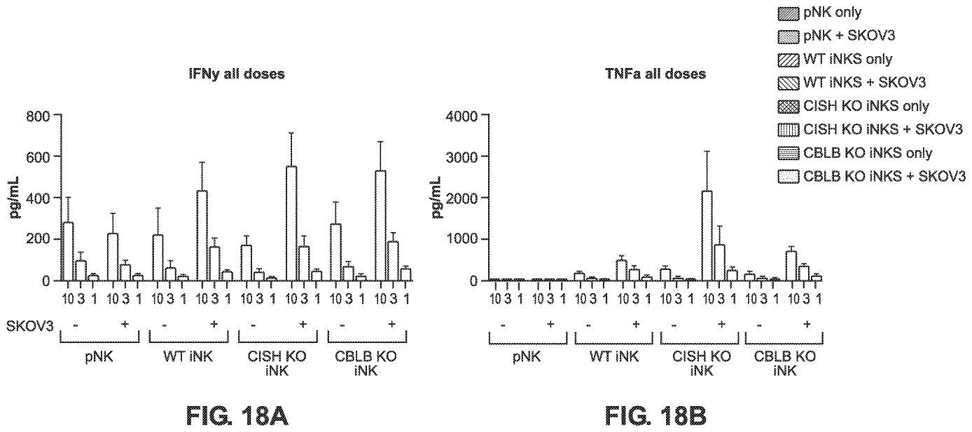

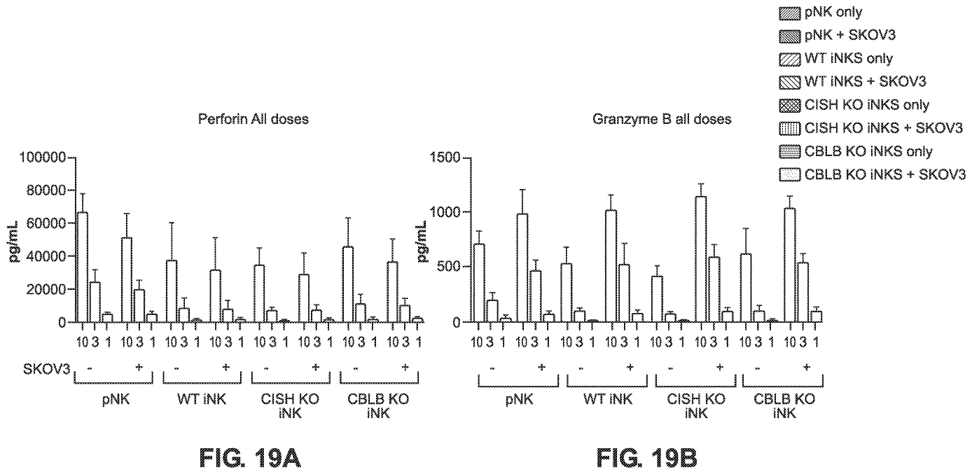

An example of measuring repeated in vitro target cell lysis (in a serial rechallenge assay) by anti-ROR1 CAR-NK cells is presented as Example 9 and FIGS. 18A-18B and 19A-19B of the instant disclosure. An example of measuring repeated in vitro target cell lysis by anti-ROR1 CAR-T cells is presented as Example 20 and FIG. 32 of the instant disclosure.

In some embodiments, the CAR-T or CAR-NK cell population exhibiting the highest level of cytokine or chemokine secretion in the presence of ROR1-expressing target cells is selected for administration to a patient.

In some embodiments, the properties of the CAR-T or CAR-NK cells are assessed in vivo and are selected from affecting characteristics of experimental animals carrying target tumor cells. In some embodiments, the target cells are tumor cells known to express ROR1 and experimental animals are mice engrafted with the tumor cells prior to being administered a dose of the anti-ROR1 CAR-T or CAR-NK cells. In some embodiments, the experimental animals are NGS mice engrafted with JeKo-1 tumor cells. In some embodiments, the assessment of CAR-T or CAR-NK cells comprises monitoring body weight, overall survival, and tumor burden of the mice engrafted with the tumor cells and administered a dose of the anti-ROR1 CAR-T or CAR-NK cells.

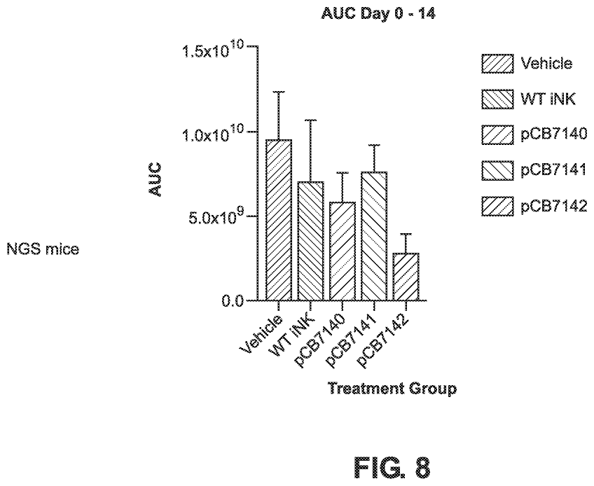

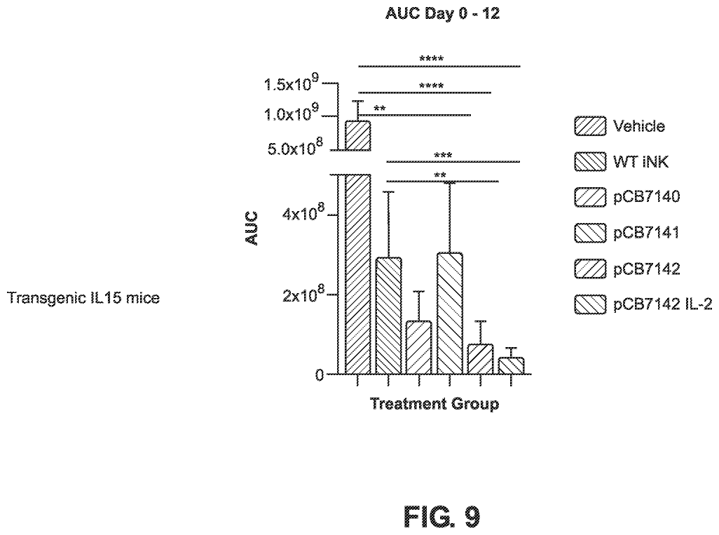

Examples of measuring in vivo reduction of tumor burden in animals with engrafted JeKo-1 tumors by the administration of anti-ROR1 CAR-NK cells are presented as Example 5 and FIG. 8, and Example 6 and FIG. 9 of the instant disclosure.

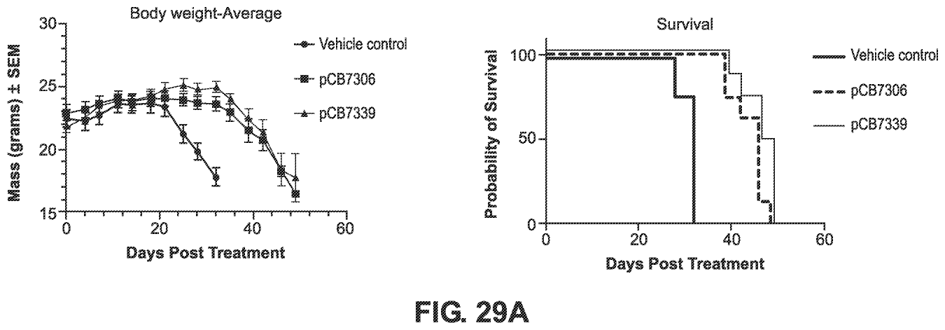

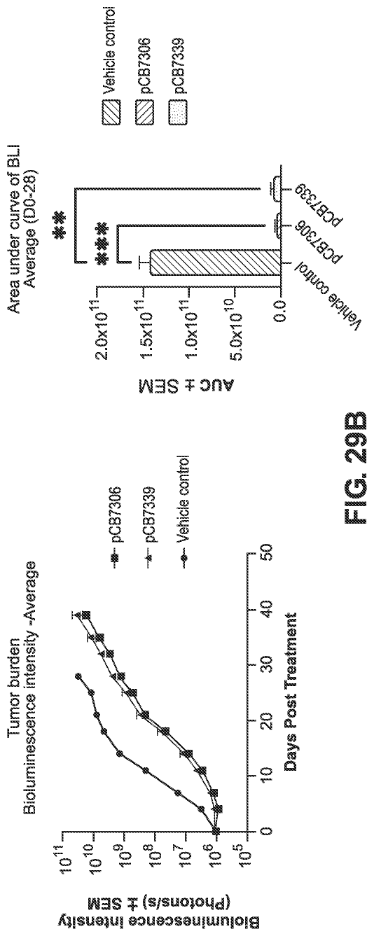

Examples of measuring in vivo reduction of tumor burden in animals with engrafted JeKo-1 tumors by the administration of anti-ROR1 CAR-T cells are presented as Example 16 and FIG. 29A-29B.

In some embodiments, the animals are engrafted with a fluorescently labeled tumor cell line and tumor burden is assessed by measuring in vivo fluorescence (and other mouse measurements). In some embodiments, the experimental animals are immunodeficient NGS mice engrafted with JeKo-1-GFPluc luciferase-expressing tumor cells. In some embodiments, the results are expressed as change in fluorescence of the tumors (or animals) over time and evaluated as area under the curve (AUC).

In some embodiments, the CAR-T or CAR-NK cell population exhibiting the most reduction in tumor burden in experimental animals engrafted with ROR1-expressing tumors and injected with the anti-ROR1 CAR-T or CAR-NK cells is selected for administration to a patient. In some embodiments, the CAR-T or CAR-NK cell population exhibiting the smallest area under the curve (AUC) is selected for administration to a patient.

In some embodiments, the assessed property is persistence of anti-ROR1 CAR-T or CAR-NK cells in the circulation of an experimental animal engrafted with a ROR1-expressing tumor and injected with the anti-ROR1 CAR-T or CAR-NK cells. In some embodiments, the persistence of anti-ROR1 CAR-NK cells is assessed as the presence and/or number of CD56⁺ cells in a volume of the animal's blood. In some embodiments, the persistence of anti-ROR1 CAR-T cells is assessed as the presence and/or number of CD8⁺ cells in a volume of the animal's blood. In some embodiments, the qualitative and/or quantitative assessment of anti-ROR1 CAR-NK cells or CAR-T cells in blood samples is performed by flow cytometry with anti-CD56 antibodies or with anti-CD8 antibodies respectively. In some embodiments, the antibodies are the anti-human CD56 antibody and the anti-human CD8 antibody.

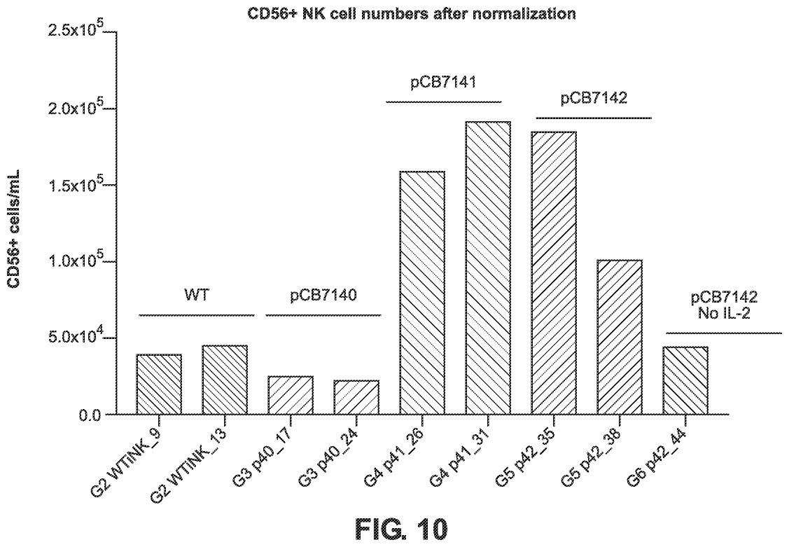

An example of measuring in vivo persistence of anti-ROR1 CAR-NK cells in circulation is presented as Example 7 and FIG. 10 of the instant disclosure.

In some embodiments, the CAR-T or CAR-NK cell population exhibiting the highest persistence in circulation of experimental animals engrafted with ROR1-expressing tumors and injected with the anti-ROR1 CAR-T or CAR-NK cells is selected for administration to a patient.

In some embodiments, the assessed property is continued expression of the anti-ROR1 CAR in the CAR-T or CAR-NK cells in the circulation of an experimental animal engrafted with a ROR1-expressing tumor and injected with the anti-ROR1 CAR-T or CAR-NK cells. In some embodiments, the expression of the CAR is assessed by flow cytometry and compared or normalized to the expression of additional NK-related genes. In some embodiments, the additional genes are one or more genes selected from CD45 (or hCD45), and CD56.

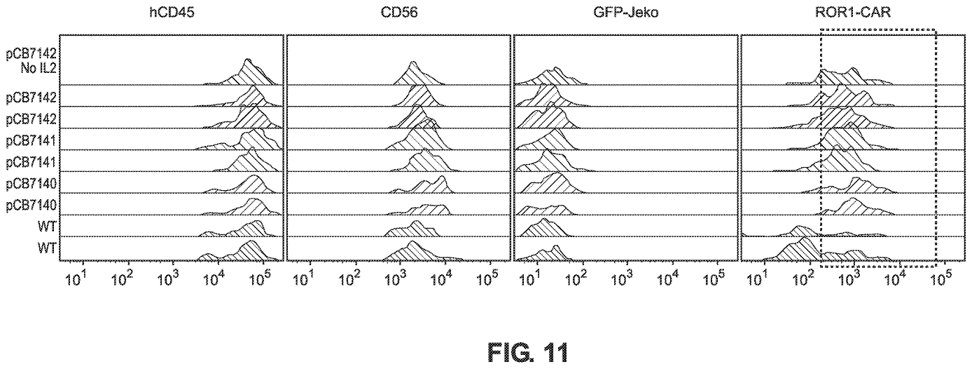

An example of measuring expression of anti-ROR1 CAR in CAR-NK cells is presented as Example 7 and FIG. 11 of the instant disclosure.

In some embodiments, the CAR-T or CAR-NK cell population exhibiting the highest level of anti-ROR1 CAR expression among the cells recovered from circulation of experimental animals engrafted with ROR1-expressing tumors and injected with the anti-ROR1 CAR-T or CAR-NK cells is selected for administration to a patient.

In some embodiments, a CAR-NK cell clone or population is selected for inclusion into the therapeutic composition described herein. The inventors have discovered that surprisingly, the anti-ROR1 CAR-NK cells with similar in vitro tumor cell killing ability exhibit substantial variation in their in vivo antitumor activity. (See FIGS. 5-6, 9-10).

In some embodiments, the CAR-T cells of the invention are compared to an existing anti-ROR1 CAR-T cells. One example of the state-of-the-art ROR1-targeting therapy is LYL797 (Spigel, D. et al., (2022, September 9-13) A ROR1-targeted CAR T-cell therapy with genetic and epigenetic reprogramming for the treatment of advanced solid tumors. Poster presentation ESMO Congress 2022, Paris, France.) The LYL797 CAR-T cells express a CAR that includes a single-chain variable fragment (scFv) derived from a high-affinity monoclonal antibody R12 binding to the amino-terminal Ig-like/Frizzled domain of ROR1. (Hudecek et al., (2013) Receptor affinity and extracellular domain modifications affect tumor recognition by ROR1-specific chimeric antigen receptor T-cells, Clin. Cancer Res. 19:3153).

In some embodiments, the CAR-T cells of the invention outperform the T cells expressing the benchmark CAR (R12) in surface CAR expression, in vitro antigen-dependent cytotoxicity, in vivo tumor control and prolongation of survival of experimental animals engrafted with ROR1-expressing tumors, see Example 26.

In some embodiments, the invention comprises compositions including anti-ROR1 CAR-T or CAR-NK cells exhibiting an anti-tumor property. In some embodiments, the invention comprises compositions including both anti-ROR1 CAR-T and CAR-NK cells exhibiting an anti-tumor property. In some embodiments, the invention comprises compositions including anti-ROR1 CAR-T or CAR-NK cells assessed for having a satisfactory property or a satisfactory level of a parameter selected from one or more of: the presence of the CAR in the cellular genome, surface expression of the CAR, antigen-dependent cytotoxicity in vitro, anti-tumor activity in vivo, antigen-dependent proliferation in vivo or in vitro, and cytokine secretion in vivo or in vitro.

Once produced and (optionally) assessed for the desired properties, the engineered cells can be formulated into compositions for delivery to a human subject to be treated. The compositions include the engineered lymphocytes, and one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and the like, and combinations thereof.

In some embodiments, the composition further comprises an antimicrobial agent for preventing or deterring microbial growth. In some embodiments, the antimicrobial agent is selected from benzalkonium chloride, benzethonium chloride, benzyl alcohol, cetylpyridinium chloride, chlorobutanol, phenol, phenylethyl alcohol, phenylmercuric nitrate, thimerosal, and the like, and combinations thereof.

In some embodiments, the composition further comprises an antioxidant added to prevent the deterioration of the lymphocytes. In some embodiments, the antioxidant is selected from ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and the like, and combinations thereof.

In some embodiments, the composition further comprises a surfactant. In some embodiments, the surfactant is selected from polysorbates, sorbitan esters, lipids, such as phospholipids (lecithin and other phosphatidylcholines), phosphatidylethanolamines, fatty acids and fatty esters; steroids, such as cholesterol, and the like.

In some embodiments, the composition further comprises a freezing agent such as 3% to 12% dimethylsulfoxide (DMSO) or 1% to 5% human albumin.

The number of anti-ROR1 CAR-T and/or CAR-NK cells in the composition will vary depending on several factors but will optimally comprise a therapeutically effective dose per vial. A therapeutically effective dose can be determined experimentally by repeated administration of increasing amounts of the anti-ROR1 CAR-T and/or CAR-NK cell-containing composition in order to determine which amount produces a clinically desired endpoint.

In some embodiments, where the subject is a human, the number of anti-ROR1 CAR-T cells per dose is fewer than about 2×10⁷ cells and the number of anti-ROR1 CAR-NK cells per dose is fewer than about 2×10⁸ cells. In some embodiments, the dose comprises 4×10⁷, 8×10⁷, 1.2×10⁸ or a suitable number in the range of 1×10⁶-2×10⁸ anti-ROR1 CAR-T cells. In some embodiments, the dose comprises 4×10⁸, 8×10⁸, 1.2×10⁹ or a suitable number in the range of 1×10⁷-2×10⁹ anti-ROR1 CAR-NK cells. In some embodiments, the dose comprises a combination of anti-ROR1 CAR-T cells and anti-ROR1 CAR-NK cells. In some embodiments, the anti-ROR1 CAR-T cells and the anti-ROR1 CAR-NK cells in the combination are present approximately at a ratio 1:10.

In some embodiments, the total number of cells in the dose is adjusted based on the percentage or CAR-expressing cells among all the cells in the cell composition. In some embodiments, the total number of cells administered is multiplied by 100/N where N is the percentage of CAR-expressing cells in the cell composition. The multiplication yields the total number of cells that must be administered to the patient in order to administer the desired number of CAR-expressing cells.

In some embodiments, the invention is a method of treating, preventing, or ameliorating a disease associated with expression of ROR1 comprising administering a population of immune cells anti-ROR1 CAR-T cells, anti-ROR1 CAR-NK cells described herein, or a combination thereof.

In some embodiments, the population of immune cells administered to a patient has been assessed for having a satisfactory property or a satisfactory level of a parameter selected from one or more of: the presence of the CAR in the cellular genome, surface expression of the CAR, antigen-dependent cytotoxicity in vitro, anti-tumor activity in vivo, antigen-dependent proliferation, and cytokine secretion.

In some embodiments, the diseases or conditions that can be treated by the immune cells of the disclosure include various malignancies including solid tumors selected from ovarian cancer, triple negative breast cancer, colorectal cancer, non-small cell lung cancer, lung adenocarcinoma, pancreatic cancer, gastric cancer, melanoma, and endometrial carcinoma, and hematological tumors selected from CLL, SLL, B-ALL, B-NHL, MCL and AML. ROR1 is an abundantly expressed tumor antigen in these types of tumors, see Balakrishnan et al. (2017) Analysis of ROR1 Protein Expression in Human Cancer and Normal Tissues, Clinical Cancer Research, 23(12):3061-3071 and Zhao et al. (2021) Tyrosine kinase ROR1 as a target in cancer therapies, Frontiers in Oncology, 11:680834.

In some embodiments, the invention is a method of inhibiting the growth of a tumor in a patient.

In some embodiments, the invention comprises a method of administering to a subject or patient a therapeutically effective number of T cells, NK cells, or a combination of T cells and NK cells expressing the anti-ROR1 CAR described herein. In some embodiments, the immune cells are pre-activated and expanded prior to administration. In some embodiment, the administration of the immune cells according to the invention results in treating, preventing, or ameliorating the disease or condition in the subject or patient. In some embodiments, the disease or disorder is selected from cancers or tumors and infections that can be treated by administration of the immune cells that elicit an immune response.

In some embodiments, the administration comprises repeated administration of the therapeutically effective number of T cells, NK cells, or a combination of T cells and NK cells expressing the anti-ROR1 CAR described herein. In some embodiments, repeated administration takes place 1, 2, 3 or more times with a one-day, a two-day, or a three or more day interval between administrations.

A pharmaceutical composition comprising T cells, NK cells, or a combination of T cells and NK cells expressing the anti-ROR1 CAR of the present disclosure can be delivered via various routes and delivery methods such as local or systemic delivery, including parenteral delivery, intramuscular, intravenous, subcutaneous, or intradermal delivery.

In some embodiments, the method comprises administering the composition of the present invention to a subject who has been preconditioned with an immunodepleting (e.g., lymphodepleting) therapy. In some embodiments, preconditioning is with lymphodepleting agents, including combinations of cyclophosphamide and fludarabine.

In some embodiments, the composition or formulation for administering to the patient is a pharmaceutical composition or formulation which permits the biological activity of an active ingredient and contains only non-toxic additional components such as pharmaceutically acceptable carriers. In some embodiments, pharmaceutically acceptable carriers include buffers, excipients, stabilizers, and preservatives.

In some embodiments, a preservative is used. In some embodiments, the preservative comprises one or more of methylparaben, propylparaben, sodium benzoate, benzalkonium chloride, antioxidants, chelating agents, parabens, chlorobutanol, phenol, and sorbic acid. In some embodiments, the preservative is present at about 0.0001% to about 2% by weight of the total composition.

In some embodiments, a carrier is used. In some embodiments, the carrier comprises a buffer, antioxidants including ascorbic acid and methionine; proteins, such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA, sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the carrier comprises a buffer. In some embodiments, the buffer comprises citric acid, sodium citrate, phosphoric acid, potassium phosphate, and various other acids and salts. In some embodiments, the buffer is present at about 0.001% to about 4% by weight of the total composition.

In some embodiments, the method comprises administering a pharmaceutical composition comprising delivery systems such that the delivery of the composition occurs over time. In such embodiments the pharmaceutical composition comprises time-release components. In some embodiments, the pharmaceutical composition comprises aluminum monostearate or gelatin. In some embodiments, the pharmaceutical composition comprises semipermeable matrices of solid hydrophobic polymers. In some embodiments, the matrices are in the form of films or microcapsules.

In some embodiments, the method comprises administering a pharmaceutical composition comprising a sterile liquid such as an isotonic aqueous solution, suspension, emulsion, dispersions, or viscous composition, which may be buffered to a selected pH. In some embodiments, the pharmaceutical composition is a sterile injectable solution prepared by incorporating the cells in a solvent such as sterile water, physiological saline, or solutions or glucose, dextrose, or the like. In some embodiments, the pharmaceutical composition further comprises dispersing, or emulsifying agents, pH buffering agents, gelling or viscosity enhancing additives, preservatives, flavoring agents, colors, and the like, depending upon the route of administration and the preparation desired.

In some embodiments, the T cells or NK cells expressing the anti-ROR1 CAR described herein are co-administered with cytokines. In some embodiments, the cytokines are selected from IL-2, IL-12, IL-15, IL-18, and IL-21. In some embodiments, the cytokines are administered at a dose per kg of body weight of a human that is equivalent to 10 ng/mouse for IL-15, 100,000 units/mouse for IL-2, and 10 μg/mouse for IL-21.

In some embodiments, the T cells or NK cells expressing the anti-ROR1 CAR described herein are engineered to constitutively express cytokines. In some embodiments, the cytokines are human cytokines. In some embodiments, the constitutively expressed cytokines are membrane-bound. In some embodiments, the constitutively expressed membrane-bound cytokine is selected from IL-15 (mbIL-15) and IL-21 (mbIL-21). In some embodiments, the constitutively expressed membrane-bound cytokine comprises a fusion of the cytokine to its receptor (cytokine-receptor fusion). In some embodiments, the membrane-bound cytokine-receptor fusion is selected from IL-15-IL-1S receptor fusion (IL-15-IL15RA fusion) and IL-21-IL-21 receptor fusion (IL-21-IL-21RA fusion). In some embodiments, the fusion also comprises a signal peptide. In some embodiments, the leader peptide is selected from IL-2 signal peptide and CD2 signal peptide.

Using a mouse model, Rowley, J. et al., ((2009) Expression of IL-15RA or an IL-15/IL-15RA fusion on CD8 T cells modifies adoptively transferred T cell function in cis. Eur. J. Immunol. 39:491) have successfully fused IL-15 with IL-15R via a serine-glycine linker, expressed the fusion in T cells, and demonstrated improved viability and proliferation of mouse CD8⁺ T cells expressing the fusion.

Fusing human IL-21 with its receptor is described in the international application Serial No. PCT/US23/67427 Cytokine-receptor fusions for immune cell stimulation filed on May 24, 2023.

In some embodiments, the coding sequence for the membrane-bound cytokine is introduced into cells via chemical or electrochemical means (such as lipid nanoparticle or electroporation). In some embodiments, the coding sequence for the membrane-bound cytokine is introduced into cells using vectors such as lentiviral vectors.

In some embodiments, a lentiviral vector includes an expression construct comprising a promoter and coding sequences for the cytokine and its receptor. In some embodiments, the cytokine and its receptor are joined by a serine-glycine linker. In some embodiments, the promoter is selected from EF1α, PGK1, MND, Ubc, CAG, CaMKIIa, β-actin, SV40 early and late promoters, the cytomegalovirus (CMV) immediate early promoter, and the Rous sarcoma virus long terminal repeat (RSV-LTR) promoter, mouse mammary tumor virus long terminal repeat (MMTV-LTR) promoter, the β-interferon promoter, and the hsp70 promoter. In some embodiments, the promoter is an EF-1α promoter.

In some embodiments, the lentiviral construct described herein is introduced into NK cells. In some embodiments, the NK cells are primary NK cells. In some embodiments, the NK cells are iNK cells (NK cells differentiated in vitro from iPSCs).

In some embodiments, the lentiviral construct described herein is introduced into iNK cell precursors such as iPSCs. In some embodiments, introduction of the lentiviral construct described herein, and the presence of the cytokine-receptor fusion does not negatively affect the ability of iPSCs to differentiate into iNK cells.

In some embodiments, the T cells or the NK (or iNK) cells are assessed for surface expression of the membrane-bound cytokine. The surface expression of the membrane-bound cytokine may be assessed by fluorescence-activated cell sorting (FACS), microfluidics-based screening, ELISA, or Western blot. In some embodiments, the surface expression of the membrane-bound cytokine is assessed by flow cytometry with an anti-cytokine antibody. In some embodiments, the T cell, or the NK (or iNK) cell population with the highest surface expression of the membrane-bound cytokine is selected for administration to a patient. In some embodiments, the iPSC cell population with the highest surface expression of the membrane-bound cytokine is selected for differentiation into iNK cells.

In some embodiments, the T cells, or the NK (or iNK) cells expressing the membrane-bound cytokine are assessed for cytotoxic properties. In some embodiments, the cytotoxic properties are assessed by in co-culturing with human tumor cells. In some embodiments, the membrane-bound cytokine-expressing NK (or iNK) cell population with the highest cytotoxic activity is selected for administration to a patient.

### EXAMPLES

### Example 1. Anti-ROR1 CAR Constructs

CAR constructs are shown in FIG. 1. The CARs contained the 3 single-chain variable regions (scFvs) described in the international application Ser. No. PCT/US2023/067314 filed on May 22, 2023, Anti-ROR1 antibody and ROR1-targeting engineered cells. The CARs contained CD8 hinge and transmembrane domains, and the cytoplasmic domain consisted of 4-1BB and CD3 zeta domains. The expression construct further comprises the minimal CAG (mCAG) CMV-derived promoter.

### Example 2. Making iNK Cells Expressing Anti-ROR1 CAR

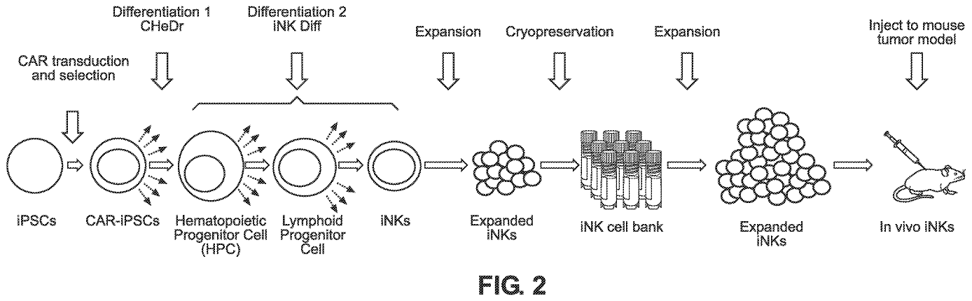

In this experiment, differentiated (induced) CAR-NK cells (CAR-iNK cells) were made according to the scheme shown in FIG. 2. CAR insertion was performed prior to the initial differentiation stage.

### CAR Insertion

Briefly, the CAR constructs (FIG. 1) were introduced into induced pluripotent stem cells (iPSCs) via lentiviral transduction (HIV-1-derived virus, (SignaGen Laboratories, Frederick, Md.) MOI 30) and selection with zeocin. Successful transduction was accessed by sorting (FACS) based on CAR staining and differentiation to iNKs. Confirmation of CAR expression and NK (iNK) differentiation stage was obtained by co-staining of anti-ROR1 CAR and CD56.

Differentiating iPSC into NK Cells

Briefly, to initiate differentiation of the iPSC clonal line, spheroids of defined size were generated and seeded onto surfaces coated with LN-511. Spheroids were grown in a medium containing a specific set of cytokines (BMP4, VEGF, SCF, IL3, IL6, and TPO) to induce differentiation into hematopoietic cells (HPCs). There are two phases of HPC differentiation (each consisting of one-week intervals) involving a different cytokine cocktail in the media.

The cells were harvested from the HPC differentiation phase, the CD34-positive fraction of cells was enriched and transferred to culture containers with an adherent irradiated first feeder cell line (AFT024—mouse fetal liver stromal cell line) cultured on the surface (seeded one day in advance). This transfer onto irradiated feeder line #1 in combination with a cytokine cocktail (IL3, IL15, IL7, SCT, FLT3L) initiates the iNK differentiation stage. The differentiating iNKs were re-plated onto fresh feeders on a weekly basis. Media changes occurred after 3 days. The iNK differentiation phase persisted for four weeks. Cell characterization was done weekly by in-process flow cytometry to test for CD45 and CD56 positivity.

After 4 weeks of iNK differentiation stage, the iNK expansion phase took place for two weeks. The iNK cells were co-cultured with the irradiated suspension of the second feeder cell line (K562 cell line engineered to overexpress 41BBL and membrane bound IL-21). After 7 days of expansion, culture cells were characterized by flow cytometry. Expanded iNKs are cryopreserved at this point. For in vitro and in vivo functional evaluation, cryopreserved iNKs are thawed, and fresh feeders are added to initiate the second week of expansion. Media changes were performed every 2-3 days. After 2 weeks of the expansion phase the final expanded iNK product was evaluated in in vitro and in vivo functional assays.

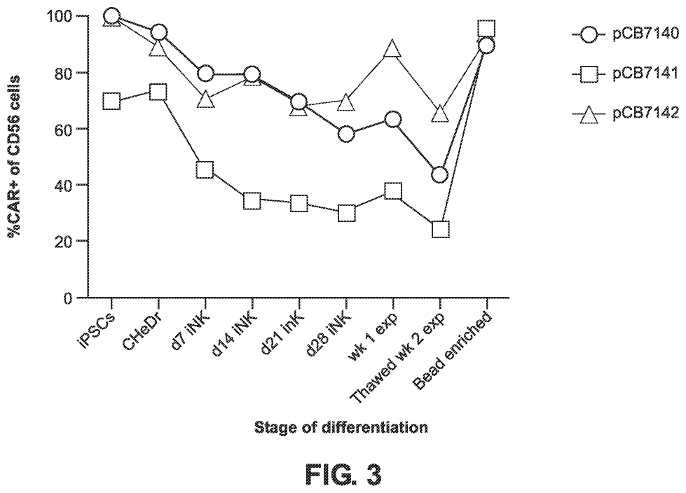

Anti-ROR1 CAR expression was measured during the differentiation process by flow cytometry, staining with ROR1-Avi tag (ACROBiosystems, Newark, Del.). FIG. 3 shows the percentage of CAR-expressing cells during each stage of iNK differentiation in cells transduced with each of the CAR constructs shown in FIG. 1.

### Example 3. In Vitro Anti-ROR1 Cytotoxicity

In this experiment, the anti-ROR1 iNK cells of Example 2 were tested in vitro for cytotoxicity against ROR1-expressing tumor cell lines SKOV3 and JeKo-1 engineered to express GFPluc. ROR1 expression in SKOV3 and JeKo-1 was assessed by flow cytometry. Results are shown in FIG. 4.

In vitro cytotoxicity was assessed by co-culturing the anti-ROR1 CAR iNK cells with the tumor cells at effector:target (E:T) ratios ranging from E:T=0.03:1 to E:T=10:1.

Results for the SKOV3 target cell line are shown in FIG. 5. The proportion of remaining live target cells after the E:T=10:1 coculture was assessed by Incucyte® Live-Cell Analysis Systems (Sartorius, AG, Gottingen, Germany), an image analysis system capable of detecting the remaining GFP⁺ SKOV3 cells.

JeKo-1-GFPluc target cells were labelled with cell trace violet dye (CTV) in advance and co-cultured with anti-ROR1 CAR iNK effector cells at a range of E:T ratios. At the analysis timepoints, 7AAD dead cell stain was added to the cultures. Assay analysis is based on the flow cytometry count of CTV⁺7AAD⁻ (live target cells) remaining, normalized to the target-only (no effector) wells. Results are shown in FIG. 6.

### Example 4. Repeated Cytotoxic Activity by Anti-ROR1 INK Cells

In this experiment, a repeat challenge (“serial rechallenge”) assay was performed on GFP-expressing JeKo-1 cell line. Briefly, plates seeded with target cells were co-cultured with anti-ROR1 CAR iNK cells. After one and two days, additional target cells were added to selected plates to test whether the iNK cells are still capable of cytotoxic response. Results are shown in FIG. 7 as specific lysis of target cells at various E:T ratios. The three lines in each chart represent the first, second, and third encounter of target cells by the iNK cells. The dashed line represents the level of cytotoxic activity of the control (no CAR) iNK cells at the first challenge with target cells.

### Example 5. In Vivo Anti-Tumor Activity of Anti-ROR1 CAR-iNK Cells in NGS Mice

In this experiment, in vivo anti-tumor activity of anti-ROR1 CAR iNK cells was assessed in NGS mice. The assay design in shown in Table 1.

TABLE 1
Assay set-up for in vivo anti-tumor activity (NGS mice).

| | Cell | # | JeKo-1- | iNK | % | | iNK | JeKo-1 |
|---|---|---|---|---|---|---|---|---|
| Group | Line* | Mice | GFPluc | dose | CAR | iNK ** | Injection | Injection |
| 1 | Vehicle | 8 | 5 × 10⁵ | | | | | IV |
| 2 | WT | 8 | 5 × 10⁵ | 2 × 10⁷ | | Day 3 | IV | IV |
| 3 | pCB7140 | 8 | 5 × 10⁵ | 2 × 10⁷ | 79.25% | Day 3 | IV | IV |
| 4 | pCB7141 | 8 | 5 × 10⁵ | 2 × 10⁷ | 92.51% | Day 3 | IV | IV |
| 5 | pCB7142 | 8 | 5 × 10⁵ | 2 × 10⁷ | 87.72% | Day 3 | IV | IV |

*iNKs with no CAR or indicated CAR
** iNK injection day post-tumor engraftment

Briefly, JeKo-1-GFPluc cells were injected intravenously (IV). At day 3 post-engraftment, effector cells were also IV-injected. The animals were dosed intraperitoneally (IP) with IL-2 (10,000 U/animal) on the iNK injection day, and every 3-4 days thereafter up to day 21. The animals were also IP-dosed with IL-15 (10 ng/animal) on the iNK injection day and daily thereafter up to day 7. Magnetic beads used for CAR enrichment were not removed prior to iNK engraftment.

Results are shown in FIG. 8 as changes in tumor burden measured as area under the curve (AUC) for each treatment group.

### Example 6. In Vivo Anti-Tumor Activity of Anti-ROR1 CAR-iNK Cells in NOG Mice Expressing Human IL-15

In this experiment, in vivo anti-tumor activity of anti-ROR1 CAR iNK cells was assessed in NOG mice expressing transgenic human IL-15. The assay design in shown in Table 2.

TABLE 2
Assay set-up for in vivo anti-tumor activity (IL-15 mice).

| | Cell | # | JeKo-1- | | | iNK | JeKo-1 | |
|---|---|---|---|---|---|---|---|---|
| Grp. | Line* | Mice | GFPluc | iNK | iNK** | Injection | Injection | % CAR |
| 1 | PBS | 8 | | | | | | |
| 2 | WT | 8 | 5 × 10⁵ | 2 × 10⁷ | Day 3 | IV | IV | |
| 3 | pCB7140 | 8 | 5 × 10⁵ | 2 × 10⁷ | Day 3 | IV | IV | 99.36% |
| 4 | pCB7141 | 8 | 5 × 10⁵ | 2 × 10⁷ | Day 3 | IV | IV | 98.47% |
| 5 | pCB7142 | 8 | 5 × 10⁵ | 2 × 10⁷ | Day 3 | IV | IV | 96.81% |
| 6 | pCB7142 | 8 | 5 × 10⁵ | 2 × 10⁷ | Day 3 | IV | IV | 96.81% |
| | (no IL2) | | | | | | | |

*iNKs with no CAR or indicated CAR
**iNK injection day post-tumor engraftment

Mice engineered to express transgenic human IL-15 (hIL-15) were used. Briefly, JeKo-1-GFPluc cells were injected intravenously (IV). At day 3 post-engraftment, effector cells were also IV-injected. The animals were dosed intraperitoneally (IP) with IL2 (10,000 U/animal) on the iNK injection day and every 3-4 days thereafter up today 21, except group 6, which received no IL2. Unlike in Example 5, no exogenous IL15 was used. Also unlike in Example 5, magnetic beads used for CAR enrichment were removed prior to iNK engraftment.

### Example 7. Persistence of Circulating Anti-ROR1 CAR-iNK Cells In Vivo

In this example, the properties of the iNK cells were assessed in treated NOG-hIL15 animals. Blood samples were collected on day 7 post-engraftment and iNKs were quantified by flow cytometry by co-staining with CD45, CD56 and the use of counting beads. Results are shown in FIG. 10. The iNK cell numbers are normalized to the volume of blood collected.

The cells where further assessed for continued expression of the anti-ROR1 CAR. Surface expression of human CD45, human CD56, GFP and the anti-ROR1 CAR was detected by flow cytometry. Results are shown in FIG. 11.

### Example 8. CBLB-Deficient Anti-ROR iNK Cells

In this example, the CBLB gene was disrupted in an iPSC cell line using CRISPR Casl2a and DNA-containing guide RNAs (chRDNAs) essentially as described in International Application Pub. No. WO2022086846 DATA-containing polynucleotides and guides for CRSIPR Type V systems, and methods of making and using the same. ChRDNAs were designed to target the CBLB locus for cleavage with Casl2a and subsequent DNA repair leading to disruption of the gene sequence and silencing (“knock-out”) of the gene.

Next, the CBLB-deficient iPSCs were differentiated into iNKs essentially as described in Example 2.

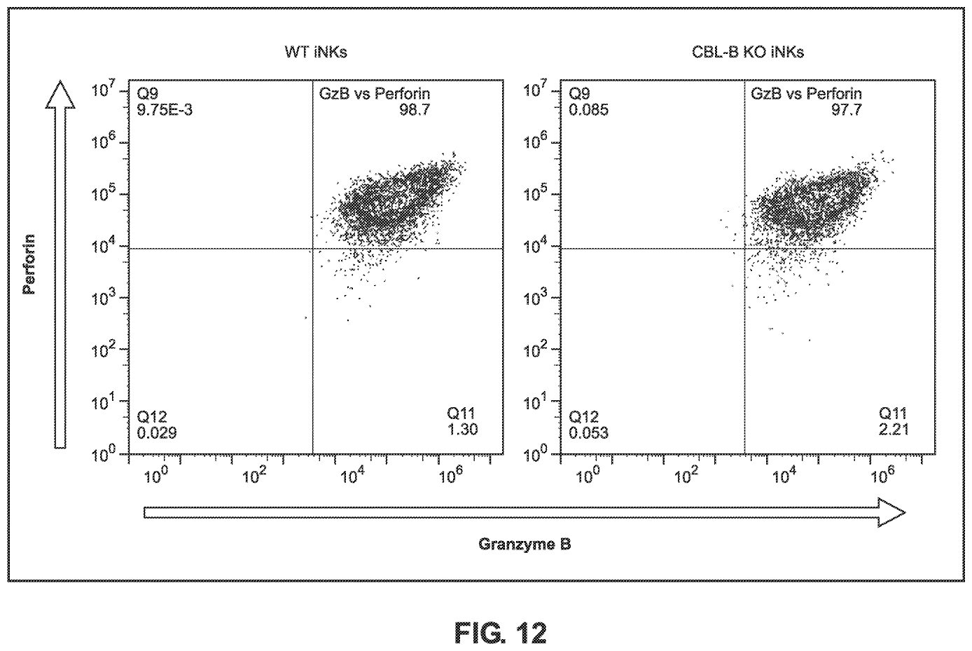

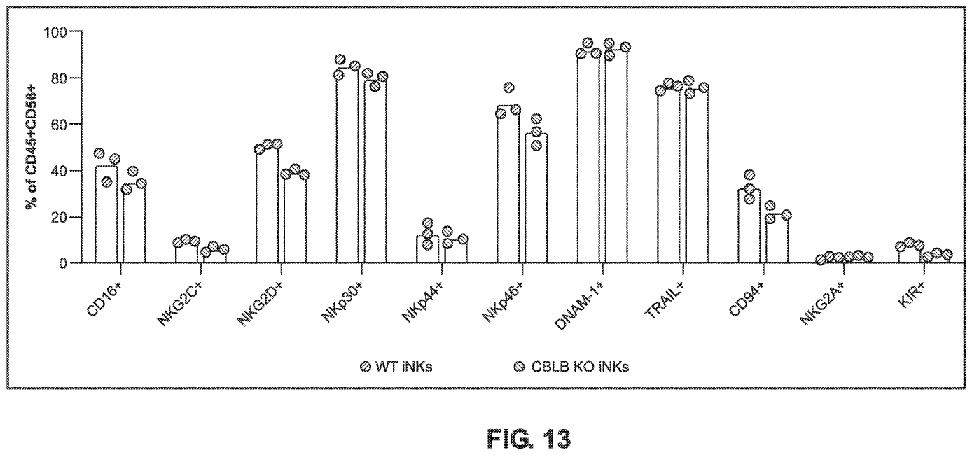

The phenotype of the resulting CBLB KO iNK cells was assessed for secretion of perforin and granzyme B by a commercial immunoassay. (FIG. 12). The phenotype was further assessed by flow cytometry by measuring levels of expression of 11 NK cell markers relative to CD45 and CD56. (FIG. 13).

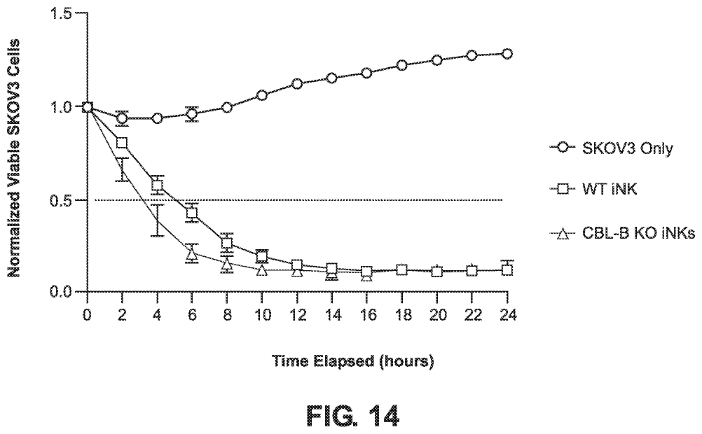

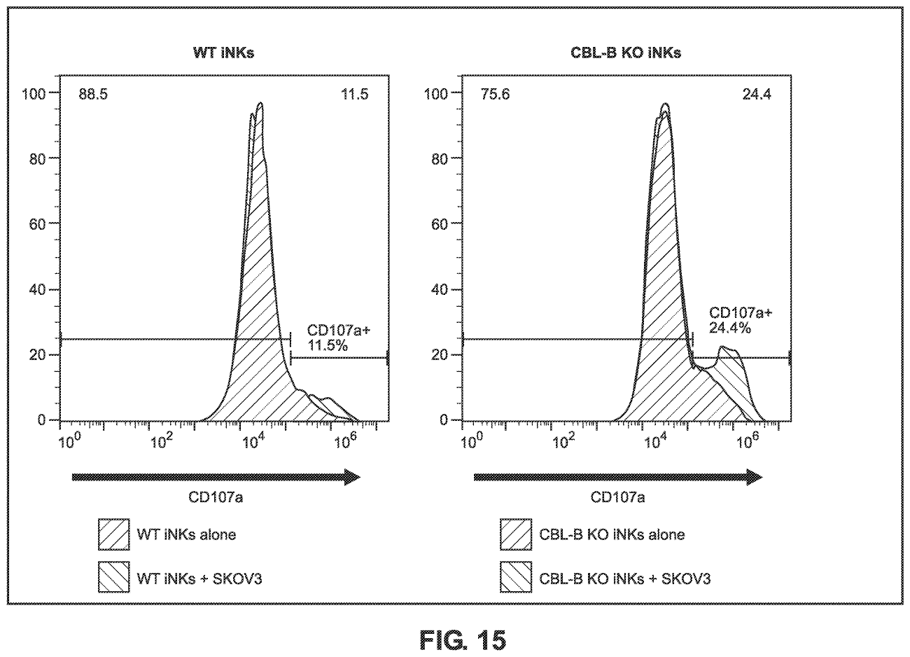

The CBLB KO iNKs were further assessed for cytotoxicity against SKOV3 ovarian tumor target cells in an in vitro co-culture essentially as described in Example 3. Cytotoxic effect of the CBLB KO iNKs is shown in FIG. 14. The CBLB KO iNK-SKOV3 cocultures were also assessed for degranulation of the iNKs (measured as release of CD107a). Results are shown in FIG. 15.

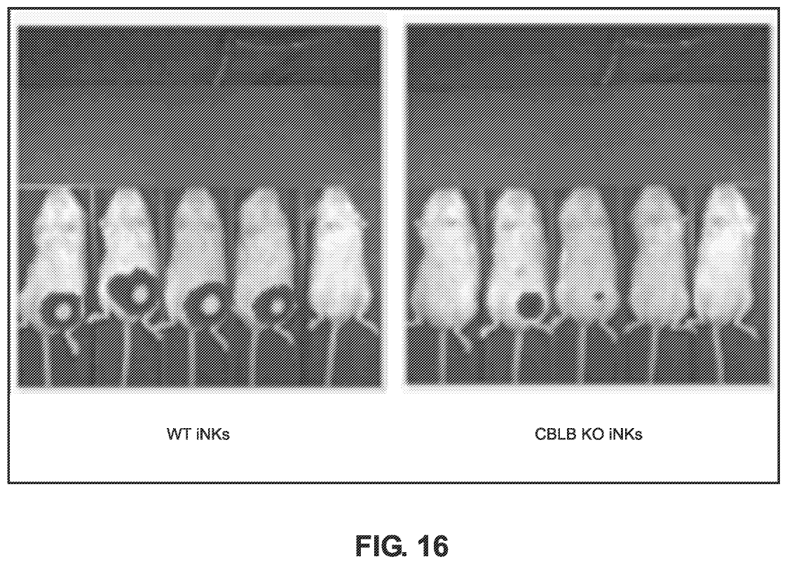

Yet further, the CBLB KO iNKs were assessed for their antitumor activity in vivo essentially described in Example 5 in SVOV3 tumor engrafted NSG mice, results are shown in FIG. 16.

### Example 9. CBLB-Deficient Anti-ROR1 iNK Cells and CISH-Deficient Anti-ROR1 iNK Cells

Disruption of the CBLB gene was described in Example 8. In this example, in a separate iPSC cell line, the CISH gene was disrupted using CRISPR Casl2a chRDNAs essentially as described in WO2022086846. ChRDNAs were designed to target the CISH locus for cleavage with Casl2a and subsequent DNA repair leading to disruption of the gene sequence and silencing (“knock-out”) of the gene. The CBLB-deficient iPSCs and the CISH-deficient iPSCs were differentiated into iNKs essentially as described in Example 2.

In Vitro Activity of the CBLB-Deficient iNKs and the CISH-Deficient iNKs

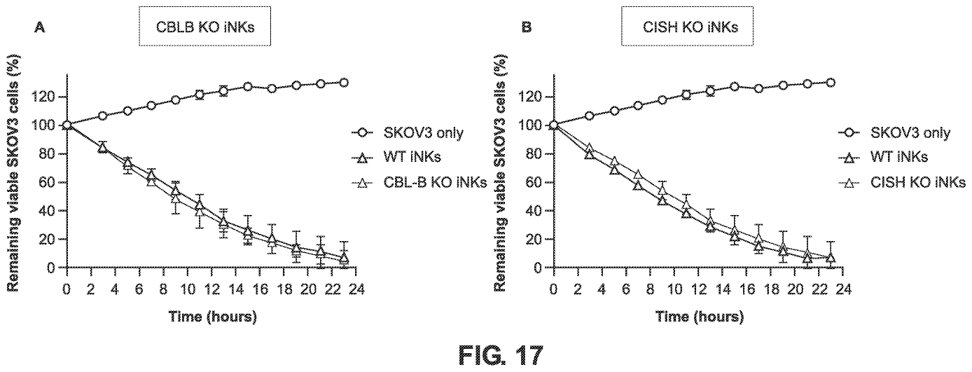

The resulting CBLB-deficient iNKs and the resulting CISH-deficient iNKs were assessed for cytotoxicity against SKOV3 cells in co-cultures essentially as described in Example 3. The CBLB KO iNKs or the CISH KO iNKs were cocultured with the SKOV3-GFPluc tumor cell line at 10:1 E:T ratio. The number of remaining viable SKOV3 cells was calculated via imaging of GFP⁺ SKOV3 target cells minus GFP⁺YO⁻PRO3⁺ labelled dying target cells, normalized to viable SKOV3 GFP+YO⁻PRO3⁻ count at time zero. Results are shown in FIG. 17.

Effector molecule secretion by the CBLB-deficient iNKs and the CISH-deficient iNKs was assessed with and without co-culture with SKOV3 ovarian tumor target cells. IFNγ, TNFα, perform and granzyme B measured following 24-hour culture of CISH KO iNKs or CBLB KO iNKs, or as controls, primary NKs (pNKs) or wild-type iNKs with or without SKOV3 target cells at 1:1 E:T ratio. The effector molecules were measured using the ProcartaPlex immunoassay on the Luminex instrument (ThermoFisher Scientific, Waltham, Mass.). Results are shown in FIG. 18 and FIG. 19.

In Vivo Anti-Tumor Activity of the CBLB KO iNKs and the CISH KO iNKs.

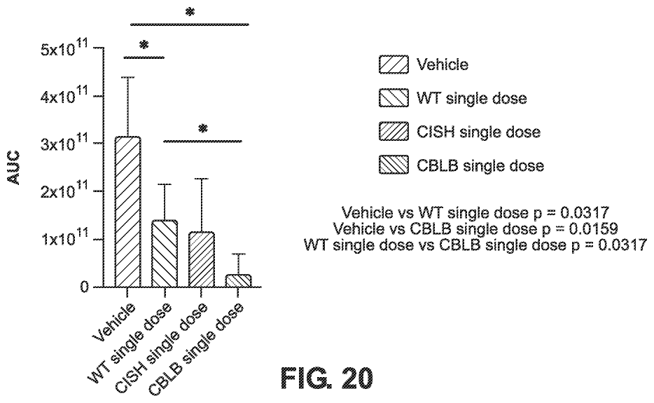

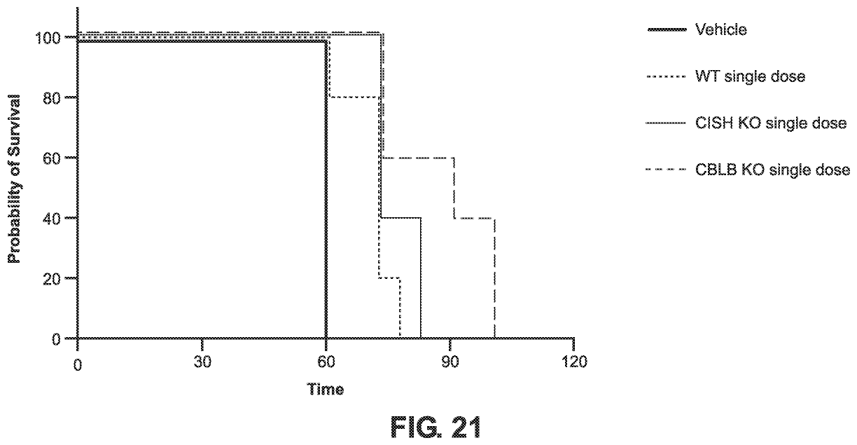

In this experiment, 3×10⁵ of SKOV3-GFPluc tumor cells were injected intraperitoneally (IP) in NSG mice at day −4. On day −1, the tumor-engrafted animals were analyzed by fluorescence imaging (IVIS® Spectrum in vivo Imaging System, Perkin Elmer, Waltham, Mass.). The animals were randomized across treatment groups. On day 0, 2×10⁷ iNKs (the CBLB KO iNKs and the CISH KO iNKs) were injected IP. The animals were imaged twice weekly thereafter up to day 45 to collect bioluminescence data. Area under the curve (AUC) analysis of bioluminescence intensity is shown in FIG. 20. The AUC analysis only covered the time when all vehicle control animals remained alive. β-value annotations*>0.05. Probability of survival for vehicle and iNK treatment groups is shown in FIG. 21.

### Example 10. Immune Cloaking of Anti-ROR1 iNK Cells

In this example, the genome of iNK cells was edited by inserting a B2M-HLA-E fusion construct into the B2M locus. In control iNK cells, the B2M locus was disrupted by CRISPR Casl2a cleavage but no construct was inserted (“B2M KO iNKs”).

Design and insertion of the B2M-HLA-E fusion construct into the B2M locus was performed essentially as described in WO2022086846. Briefly, the fusion nucleic acid construct encoded in the N—C orientation, an N-terminal B2M secretion signal, an HLA-G derived peptide sequence, a first linker sequence, the B2M sequence, a second linker sequence, and an HLA-E sequence. The nucleic acid construct further contained an EF1α mammalian promoter sequence and a C-terminal BGH polyadenylation signal sequence.

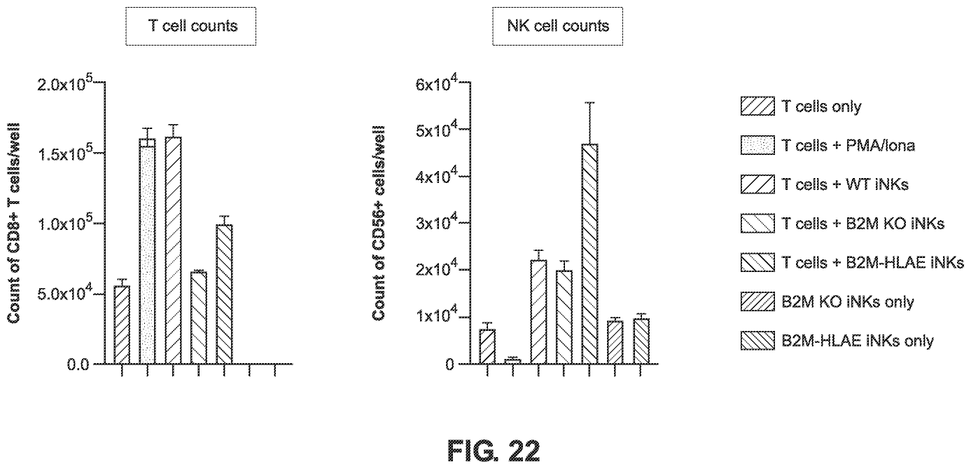

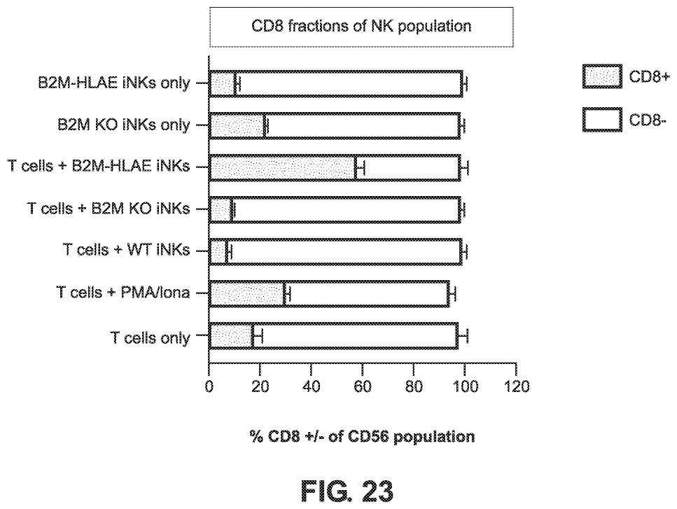

The resulting edited iNK cells were tested for cytotoxic properties and survival in co-cultures with allogeneic T cells. CD8⁺ T cells were obtained by enrichment by negative selection from donor derived PBMCs. The T cells were co-cultured with wild-type iNKs, B2M KO iNKs and B2M-HLA-E iNKs at the effector:target (E:T) ratio of 4:1 for 6 days. In a positive control culture, the T cells were stimulated by PMA/ionomycin. At day 6, T cells in all cultures were counted by CD8 staining and counting beads. Results are shown in FIG. 22 (“T cell counts”). Also at day 6, iNK cells were counted in iNK-containing cultures by CD56 staining and counting beads. Results are shown in FIG. 22 (“NK cell counts”). The proportion of CD8⁺ population within the CD56⁺ population of iNK cells was also determined. (FIG. 23).

### Example 11. Cytokine Expressing Anti-ROR1 iNK's

This example describes iNKs engineered to constitutively express membrane-bound cytokine IL-15 (mbIL-15).

a. Producing mbIL-15 Expressing iNKs

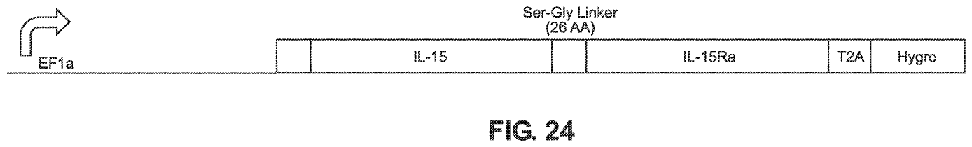

A lentiviral construct was designed to transduce iPSCs and differentiate the engineered iPSCs into iNK cells. Design of the lentiviral construct for expressing membrane-bound IL-15 (mbIL-15) is shown in FIG. 24. The expression construct inserted into the lentiviral vector contains an EF-1α promoter, and coding sequences for IL-15 and IL-15 receptor (IL-15RA) joined by a serine-glycine linker. The fusion also comprises a CD2 signal sequence. Transduction of iPSCs with the lentiviral construct was performed as described in Example 2. The iPSCs were then selected for utilizing a cell sorter (SH800, Sony Biotechnology, San Jose, Cal.) which sorted IL-15 positive cells using an anti-IL-15 primary antibody and the anti-mouse Alexa Fluor 488-conjugated secondary antibody.

Once selected, the mbIL-15-expressing iPSC clones were differentiated into iNK cells following a differentiation protocol involving three stages: a two-week stage that differentiates iPSCs into HPCs, a four-week stage that differentiates HPCs into iNKs that have been positively selected for CD34⁺, and a two-week expansion stage.

b. Cytotoxic Properties of mblL-15 Expressing iNKs

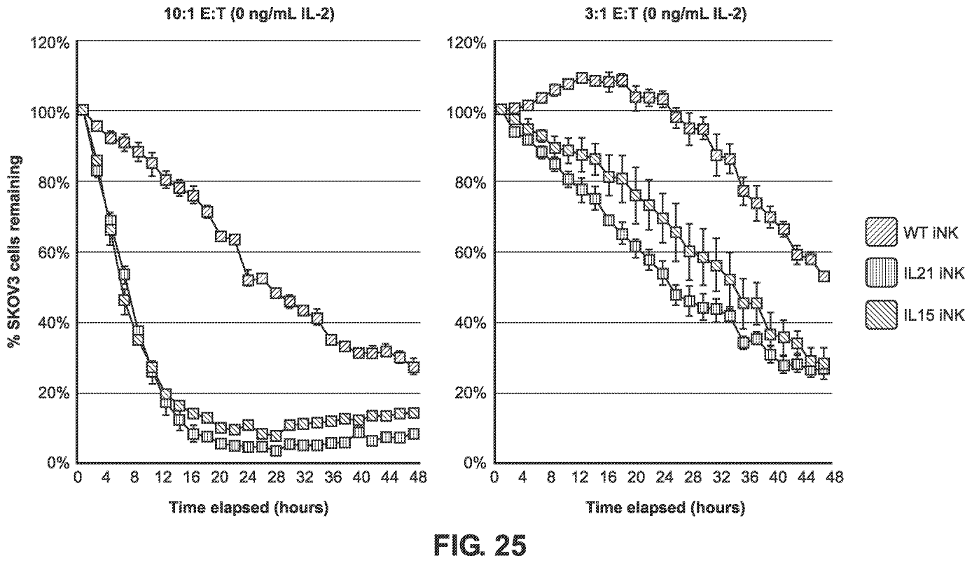

The engineered iNKs were tested for cytotoxic properties in co-culture with SKOV-3 ovarian tumor cells expressing Firefly Luciferase-GFP fusion protein as targets. Control co-cultures included wild-type iNKs. The cytotoxicity assay was performed on an Incucyte® Live-Cell Analysis Systems (Sartorius, AG, Goettingen, Germany). The co-cultures had E:T ratios of 10:1 and 3:1 and were imaged every 2 hours. The co-cultures included YO-PRO-3 viability dye (ThermoFisher Scientific, Waltham, Mass). The percentage of viable SKOV-3 cells was calculated as an overlay of the percent total SKOV-3 cells from the start of the assay. Results are shown in FIG. 25.

### Example 12. Optimizing the Anti-ROR1 scFv by Varying Linker Length

In this experiment a previously disclosed humanized single-chain variable fragment (scFv) (international application Ser. No. PCT/US2023/067314 filed on May 22, 2023, Anti-ROR1 antibody and ROR1-targeting engineered cells) was optimized by varying the length of the linker between the light and heavy chains of the scFv. The amino acid sequence of the scFv 862 disclosed herein as SEQ ID NO: 12 contains a linker consisting of four repeats of glycine-serine linker G₄S (G₄S)₄. A variant of this sequence comprised a single copy of the linker sequence (G₄S)₁. The CARs containing the two scFvs were designed as shown in FIG. 26. The CARs contained CD8A-derived signal peptide (sp), hinge (h) and transmembrane domains (tm), and the cytoplasmic domain of the CAR consisted of 4-1BB and CD3 zeta domains. The CAR expression constructs included the EF1α promoter. The original-design CAR was present in the plasmid construct pCB7306, and the short linker CAR was present in the plasmid pCB7339.

### Example 13. Insertion of the Anti-ROR1 CAR into T Cells

In this experiment the CAR designs shown in FIG. 26 were introduced into T cells for insertion into the TRAC locus. The TRAC locus was targeted using CRISPR Casl2a and CRISPR hybrid RNA-DNA guides (chRDNAs) essentially as described in International Application Pub. No. WO2022086846 DNA-containing polynucleotides and guides for CRSIPR Type V systems, and methods of making and using the same. To target the TRAC locus the exons were analyzed for the presence of a suitable PAM sequence for the Type V Acidaminococcus spp. Casl2a. The target site was chosen in the TRAC locus on human chromosome 14 between nucleotides 22547529 and 22547552. The stretch of about 20 nucleotides 3′ of the PAM sequence was used to design a targeting region of the Casl2a guides.

To design a donor sequence for insertion, 500 bp long homology arms 5′ and 3′ of the cut site were identified. The 5′ and 3′ homology arms were appended to the end of the DNA donor containing the CARs (Example 12) in a reverse orientation (i.e., 3′ to 5′) relative to the homology arms. The nucleotide sequences of the DNA donor polynucleotides were provided to a commercial manufacturer for synthesis into a suitable recombinant AAV (rAAV) plasmid and packaging into AAV6 viruses.

Primary T Cell Transduction with rAAV

Primary activated T cells were obtained from healthy donor PBMCs and transfected with rAAV6 essentially as described in WO2022086846. Briefly, T cells were electroporated with TRAC-targeting Casl2a chRDNA guide nucleoprotein complexes, and between 1 minute and 4 hours after nucleofection, cells were infected with the rAAV6 virus packaged with CAR donor sequences at an MOI of 4×10⁵. T cells were cultured in ImmunoCult-XF complete medium (STEMCELL Technologies, Cambridge, Mass.) supplemented with IL-2 (100 units/mL) for 24 hours after the transductions. The next day, the transduced T cells were transferred to 50 mL conical tubes and centrifuged at 300×g for approximately 7-10 minutes to pellet the cells. The supernatant was discarded, and the pellet was gently resuspended, and the T cells pooled in an appropriate volume of ImmunoCult-XF complete medium supplemented with IL-2 (100 units/mL). The enumerated T cells were resuspended at 1×10⁶ cells/mL in ImmunoCult-XF complete medium supplemented with IL-2 (100 units/mL) and plated into as many T-175 suspension flasks as required (max volume per flask was 125 mL). Anti-ROR1 CAR expression was assessed by flow cytometry staining with PE-Labeled Human ROR1 Protein, His-Tag (Site-specific conjugation) (ACROBiosystems, Newark, Del.).

### Example 14. In Vitro Anti-ROR1 Cytotoxicity of Anti-ROR1 CAR-T Cells

In this experiment the anti-ROR1 CAR-T cells of Example 13 were tested in vitro for cytotoxicity against ROR1-expressing targets tumor cell lines JeKo-1, REC1, and Mino, and primary B cell chronic lymphocytic leukemia (B-CLL) cells (donor PBMC fractions). A control effector T cell population had a disrupted TRAC locus but no CAR insertion (“TRAC KO”).

In vitro cytotoxicity was assessed by co-culturing the anti-ROR1 CAR-T cells with the tumor cells at effector:target (E:T) ratios ranging from E:T=0.1:1 to E:T=10:1. The target cells were labelled with cell trace violet dye (CTV) in advance and co-cultured with anti-ROR1 CAR-T effector cells for 48 hours. At the analysis timepoints, Propidium Iodide (PI) dead cell stain was added to the cultures. Assay analysis is based on the flow cytometry count of CTV⁺PI⁻ (live target cells) remaining, normalized to the target-only (E:T=0:1) wells. Results are shown as percentage of specific cell lysis in FIG. 27 A (Jeko-1 And REC1), FIG. 27 B (Mino and B-CLL, donor 1), and FIG. 27 C (B-CLL, donor 2).

### Example 15. In Vitro Cytokine Secretion by Anti-ROR1 CAR-T Cells

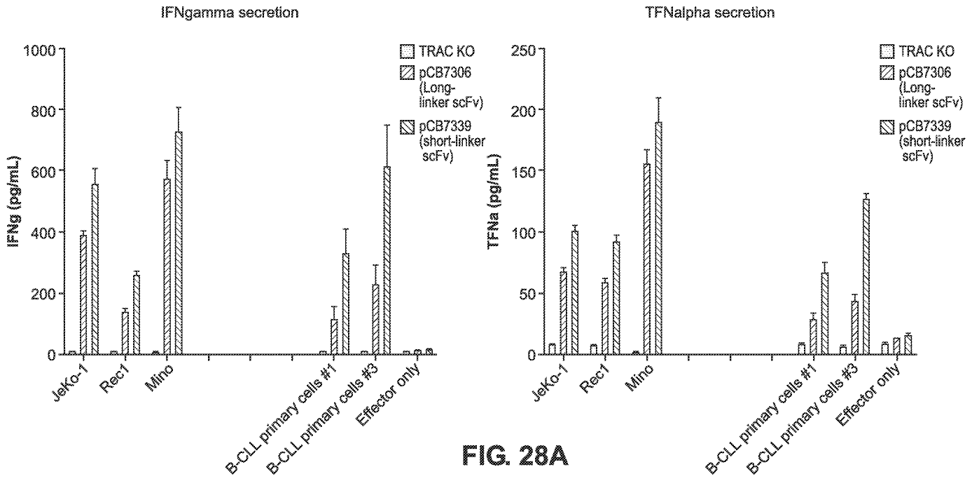

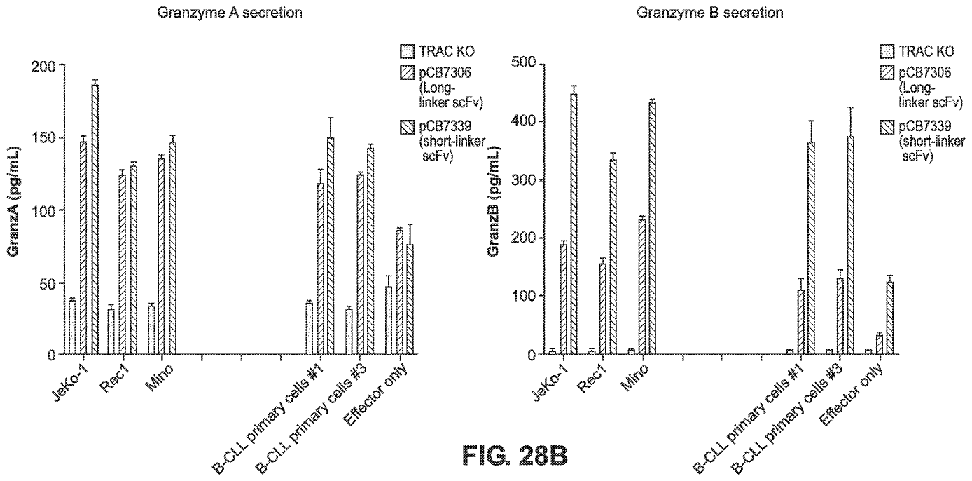

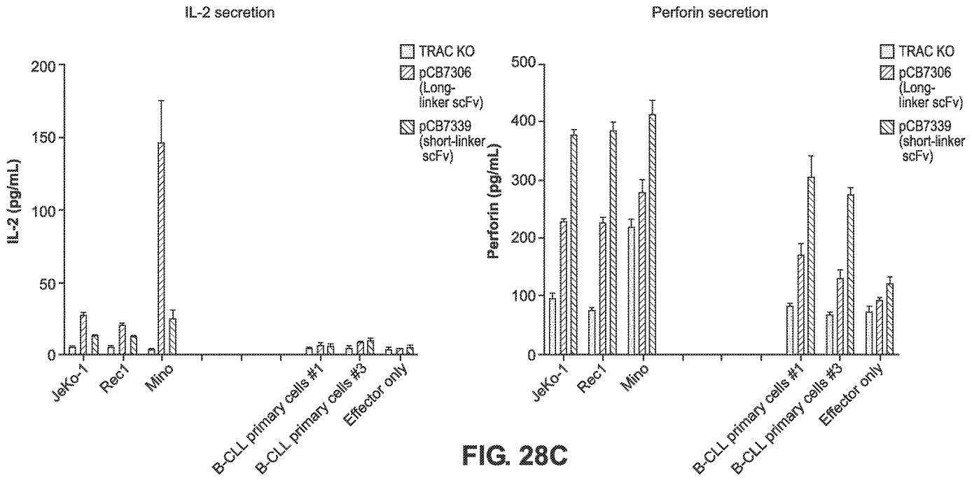

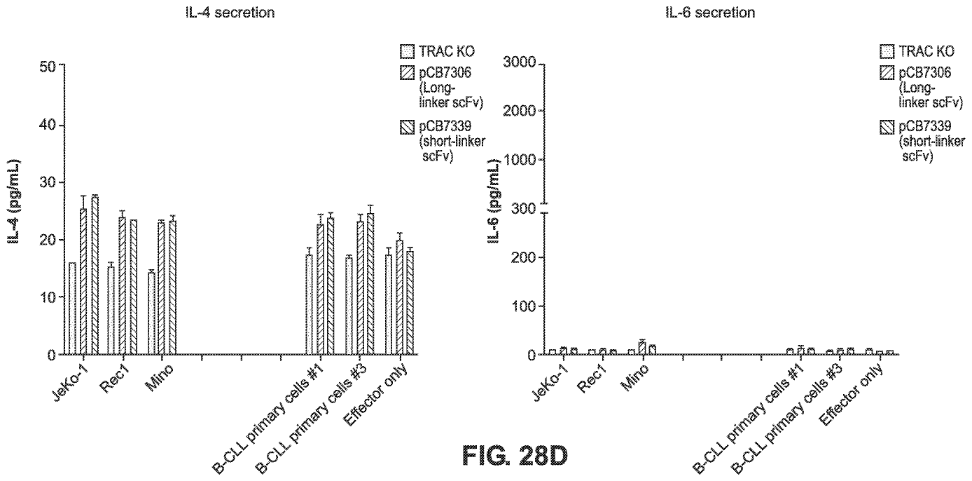

In this experiment the cocultures of CAR-T cells and ROR1-expressing targets described in Example 14 were assessed for cytokine secretion. Secretion of Interferon γ (IFNγ), Tumor Necrosis Factor α (TNFα), Granzyme A, Granzyme B, Perforin, IL-2, IL-4, and IL-6 was measured by collecting supernatants from co-cultures (1:1 E:T ratio) at the 24 hr time point. Levels of the secreted cytokines were quantified using a Luminex-based multiplex assay. Results are shown as concentration of cytokines in the co-culture supernatant in FIGS. 28 A (IFNγ and TNFα), FIG. 28 B (Granzyme A and Granzyme B), FIG. 28 C (IL-2 and Perforin), and FIG. 28 D (IL-4 and IL-6).

### Example 16. In Vivo Anti-Tumor Activity of Anti-ROR1 CAR-T Cells in NGS Mice

In this experiment in vivo anti-tumor activity of anti-ROR1 CAR-T cells (Example 13) was assessed in NSG mice. The assay design in shown in Table 1.

TABLE 1
Assay set-up for in vivo anti-tumor activity (NSG mice).

| | Cell | | JeKo- | T cell dose | | | T cell | JeKo-1 |
|---|---|---|---|---|---|---|---|---|
| Group | Line | # Mice | 1-GFPluc | (# CAR + Live+) | % CAR | T cells | Injection | Injection |
| 1 | Vehicle | 8 | 5 × 10⁵ | N/A | N/A | N/A | N/A | IV |
| 2 | pCB7306 | 8 | 5 × 10⁵ | 1 × 10⁷ | 82.0% | Day 3 | IV | IV |
| 3 | pCB7339 | 8 | 5 × 10⁵ | 1 × 10⁷ | 86.0% | Day 3 | IV | IV |

Briefly, JeKo-1-GFPluc cells were injected intravenously (IV). At day 3 post-engraftment, effector cells were also IV-injected.

The in vivo antitumor activity of the anti-ROR1 CAR-T cells is measured as changes in body weight, survival and changes in tumor burden measured as bioluminescence intensity and as area under the curve (AUC) of bioluminescence intensity for each treatment group. Results are shown in FIG. 29A (changes in body weight and probability of survival), and FIG. 29B (tumor burden measured as bioluminescence and AUC of bioluminescence (BLI) measurements). FIG. 29A: Kaplan-Meier survival curve comparisons were analyzed with the log-rank Mantel-Cox tests. Vehicle control vs. pCB7306 p=0.0002, Vehicle control vs pCB7339 p=0.0002. Statistical differences between mouse body weight at Day 28 were determined using a Kruskal-Wallis test followed by Dunn's post hoc test for multiple comparisons. Vehicle control vs pCB7306 p=0.0218 (***), Vehicle control vs pCB7339 p=0.0006 (**). FIG. 29B: To compare the BLI from Day 0 to Day 28 for each treatment group, a Kruskal-Wallis test followed by Dunn's post hoc test for multiple comparisons was applied to the AUC. Vehicle control vs pCB7306 p=0.0004, Vehicle control vs pCB7339 p=0.0042.

### Example 17. Checkpoint-Deficient Anti-ROR1 CAR-T Cells

In this experiment one of the immune checkpoint genes PDCD1, TIGIT, TIM3 and LAG3 was disrupted in CAR-T cells of Example 13. Gene inactivation using CRISPR Casl2a and CRISPR hybrid RNA-DNA guides (chRDNAs) was performed essentially as described in International Application Pub. No. WO2022086846 DNA-containing polynucleotides and guides for CRSIPR Type V systems, and methods of making and using the same. Briefly, CRISPR hybrid RNA-DNA guides (chRDNAs) were designed to target the gene locus for cleavage with Casl2a and subsequent DNA repair leading to disruption of the gene sequence, and silencing (“knock-out”) of the gene. To target each gene, the exons were analyzed for the presence of a suitable PAM sequence for the Type V Acidaminococcus spp. Cas12a. The stretch of about 20 nucleotides 3′ of the PAM sequence was used to design a targeting region of Casl2a guides for each gene.

To target the PDCD1 gene, a target site was chosen in the PDCD1 locus on human chromosome 2 between nucleotides 241852860 and 241852883. To target the TIGIT gene, a target site was chosen in the TIGIT locus on human chromosome 3 between nucleotides 114299581 and 114299604. To target the TIM3 gene, a target site was chosen in the TIM3 locus on human chromosome 5 between nucleotides 157104679 and 157104702. To target the LAG3 gene, a target site was chosen in the LAG3 locus on human chromosome 12 between nucleotides 6773330 and 6773353.

The gene-targeting Cas12a chRDNA guide nucleoprotein complexes were formed and introduced into anti-ROR1 CAR-T cells (Example 13) essentially as described in WO2022086846 to generate gene knockouts. The resulting cell populations were designated anti-ROR1 CAR+PD1 KO, anti-ROR1 CAR+TIM3 KO, anti-ROR1 CAR+LAG3 KO, and anti-ROR1 CAR+TIGIT KO respectively. Cell populations with multiple checkpoint inactivations were anti-ROR1 CAR+PD1 KO+TIM3 KO, anti-ROR1 CAR+PD1 KO+LAG3 KO, anti-ROR1 CAR+PD1 KO+TIGIT KO, and anti-ROR1 CAR+TIM3 KO+LAG3 KO,

### Example 18. In Vitro Cytotoxicity of Checkpoint-Deficient Anti-ROR1 CAR-T Cells

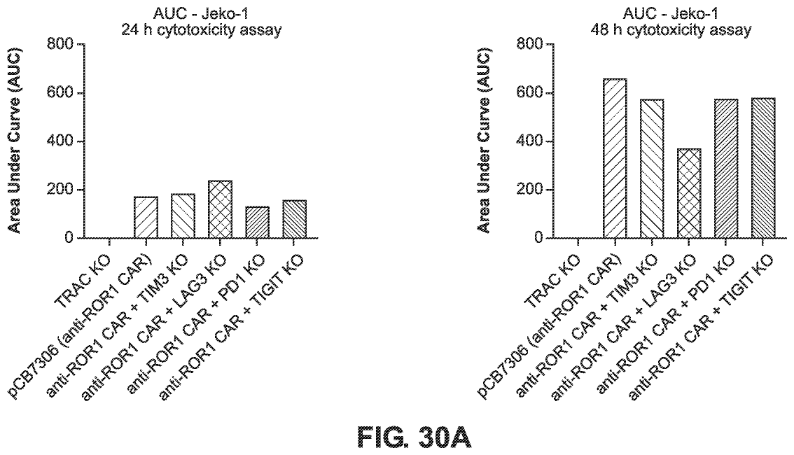

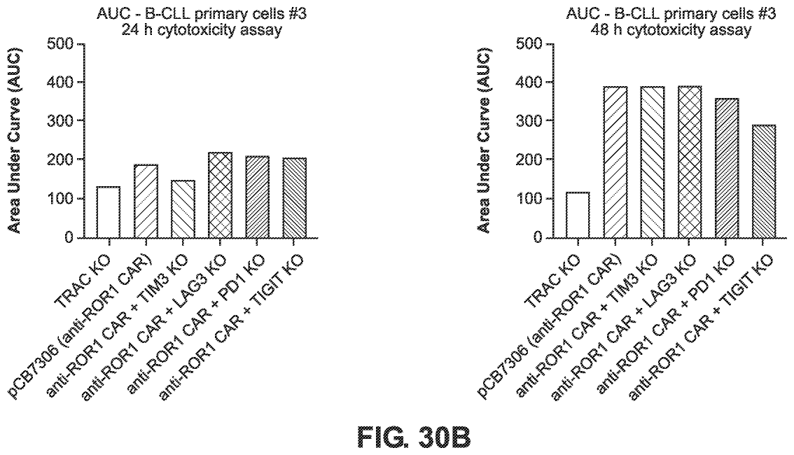

In this experiment the checkpoint-deficient anti-ROR1 CAR-T cells of Example 17 were tested for in vitro cytotoxicity against ROR1-expressing targets JeKo-1 cell line and primary B-CLL donor cells essentially as described in Example 14. For single checkpoint inactivations, results are shown in FIG. 30A (JeKo-1 cells) and FIG. 30B (primary B-CLL cells). For double checkpoint inactivations, results are shown in FIG. 33 (JeKo-1 cells and primary B-CLL cells).

### Example 19. In Vitro Cytokine Secretion by Checkpoint-Deficient Anti-ROR1 CAR-T Cells

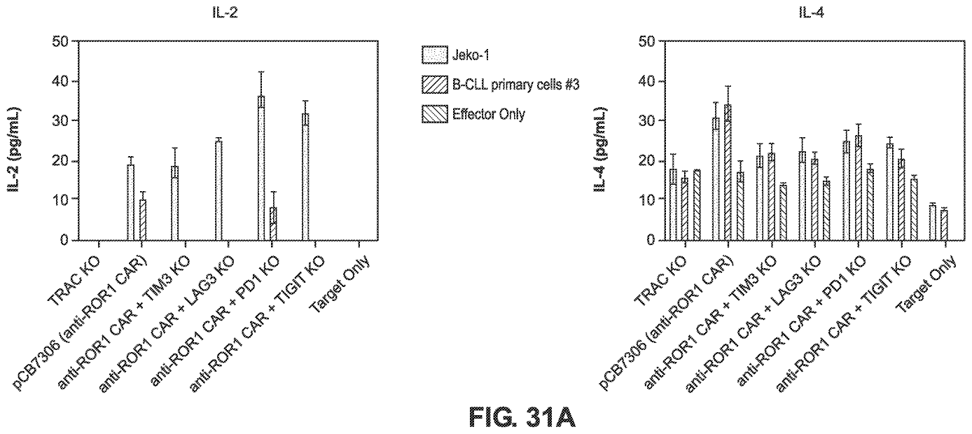

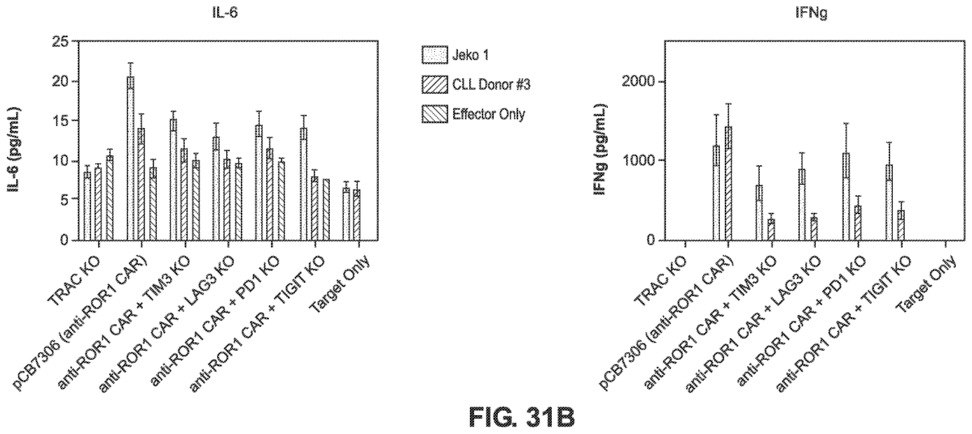

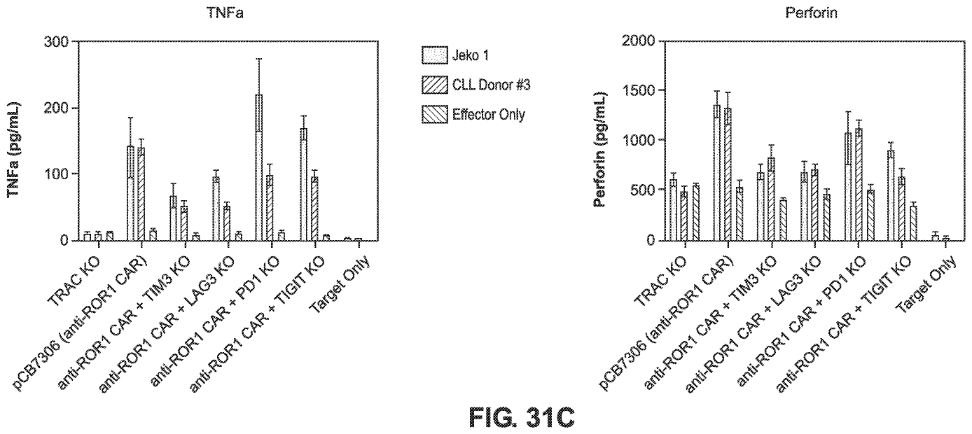

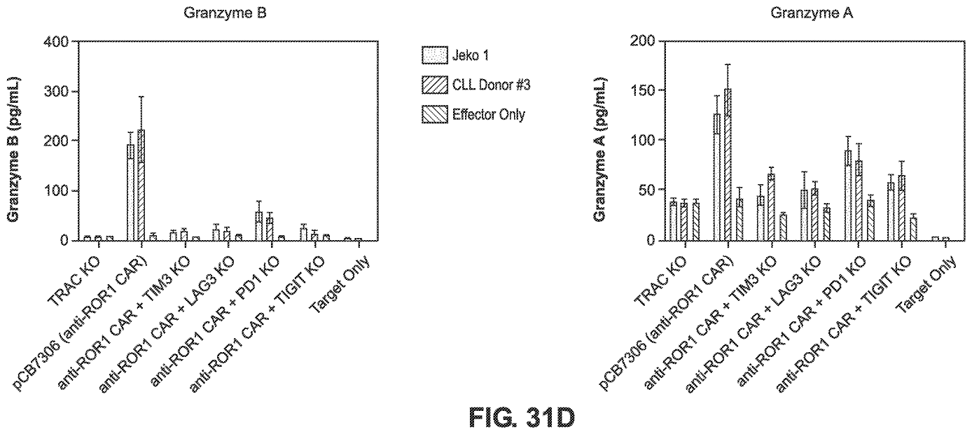

In this experiment the cocultures of checkpoint-deficient anti-ROR1 CAR-T cells and ROR1-expressing targets described in Example 18 were assessed for cytokine secretion. Secretion of Interferon γ (IFNγ), Tumor Necrosis Factor α (TNFα), Granzyme A, Granzyme B, Perforin, IL-2, IL-4, and IL-6 was measured by collecting supernatants from co-cultures at the 24 hr time point. Levels of the secreted cytokines were quantified using a Luminex-based multiplex assay. Results are shown in FIG. 31A (IL-2 and IL-4), FIG. 31B (IL-6 and IFNγ), FIG. 31C (TNFα and perforin), FIG. 31D (Granzyme A and Granzyme B).

### Example 20. Serial Rechallenge of Checkpoint-Deficient Anti-ROR1 CAR-T Cells with ROR1-Expressing Tumor Cells

In this experiment in vitro cytotoxicity of checkpoint-deficient anti-ROR1 CAR-T cells of Example 17 was assessed in a serial rechallenges with ROR1-expressing target cell line JeKo-1. Target cells, which are engineered to express luciferase, were added to the effector cells at increasing E:T ratios in the range of 0:1 to 2:1. 15,000 fresh target cells were added to each culture every 3-4 days (up to 6 rechallenges) and live cell numbers were assessed by a luminescence readout. Cytotoxicity (cell lysis) was measured after challenges 1-6 with ROR1-expressing target cells and used to calculate area under the curve AUC. Results are shown in FIG. 32.

### Example 21. Alternative Anti-ROR1CAR Designs

TABLE 3
List of CAR constructs

| Construct | Prom | S | scFv | H | TM | CS1 | CS2 | SD | Cytokine |
|---|---|---|---|---|---|---|---|---|---|
| pCB7429 | MND | CD8a | 862 | CD28 | CD28 | CD28 | IL-2Rb | CD3ζ* | N/A |
| pCB7430 | EF1 | CD8a | 862 | CD28 | CD28 | CD28 | IL-2Rb | CD3ζ* | N/A |
| pCB7431 | EF1 | CD8a | 862 | CD8 | CD8 | 4-1BB | IL-2Rb | CD3ζ* | N/A |
| pCB7432 | MND | CD8a | 862 | CD8 | CD8 | 4-1BB | IL-2Rb | CD3ζ* | N/A |
| pCB7433 | EF1 | CD8a | 862 | CD8 | CD8 | 4-1BB | N/A | CD3ζ* | N/A |
| pCB7434 | MND | CD8a | 862 | CD8 | CD8 | 4-1BB | N/A | CD3ζ* | N/A |
| pCB7436 | EF1 | CD8a | 862 | CD28 | CD28 | CD28 | N/A | CD3ζ | IL-36γ-IL2SP |
| pCB7437 | EF1 | CD8a | 862 | CD28 | CD28 | CD28 | N/A | CD3ζ | IL-36γ-IL36SP |
| pCB7438 | EF1 | CD8a | 862 | CD28 | CD28 | CD28 | N/A | CD3ζ | IL-36γ-IL2mutSP |

Abbreviations:
CD3ζ* CD3ζ with YRHQ, the STAT3 association motif
Prom Promoter
S Signal peptide
scFv scFv PMC862 (SEQ ID NO: 9)
H Hinge domain
TM Transmembrane domain
CS1 Co-stimulatory domain 1
CS2 Co-stimulatory domain 2
SD Signaling domain
IL36SP IL36 signal peptide
IL2SP IL2 signal peptide
IL2mutSP IL2 signal peptide with modifications in the basic and the hydrophobic domains (SEQ ID NO: 48)

### Example 22. Antigen-Dependent Cytotoxicity of Alternative Anti-ROR1 CAR-Design CAR-T Cells Against ROR1-Expressing Targets

In this example, the CAR-T cells of Example 21 were tested for ROR1-specific cytotoxicity in co-cultures with ROR1-positive cell line JeKo1 and ROR1-positive primary B cells from patients suffering from B-CLL. ROR1 positivity was determined by staining cells with phycoerythrin (PE)-conjugated anti-ROR1 antibody (BioLegend, San Diego, Cal., Clone 2A2) and quantifying the number anti-ROR1 antibody bound per cell thus number of ROR1 molecules per cell using BD PE Quantibrite™ beads according to manufacturer's instructions.

Target cells were labelled with Cell Trace Violet™ (CTV) stain. Anti-ROR1 CAR-Ts were cocultured with CTV-labelled target cells at various effector:target (E:T) ratios in triplicate for 48 hours. After 48 hours, propidium iodide (PI) dead cell stain was added to each well and PI uptake was evaluated via flow cytometry to quantify target cell death. Live target cell counts were extracted, and specific lysis was calculated relative to 0:1 E:T controls using the formula:

$$1 - \frac{live C T {V}^{+} target cell count in X : Y E : T ratio}{live C T {V}^{+} target cell count in 0 : 1 E : T ratio} \times 100 \%$$

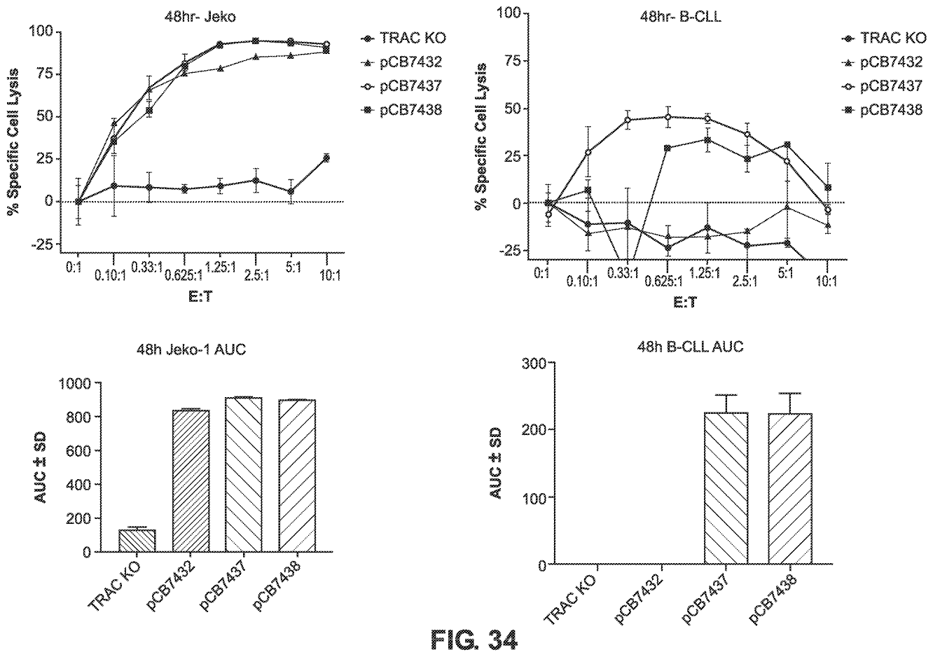

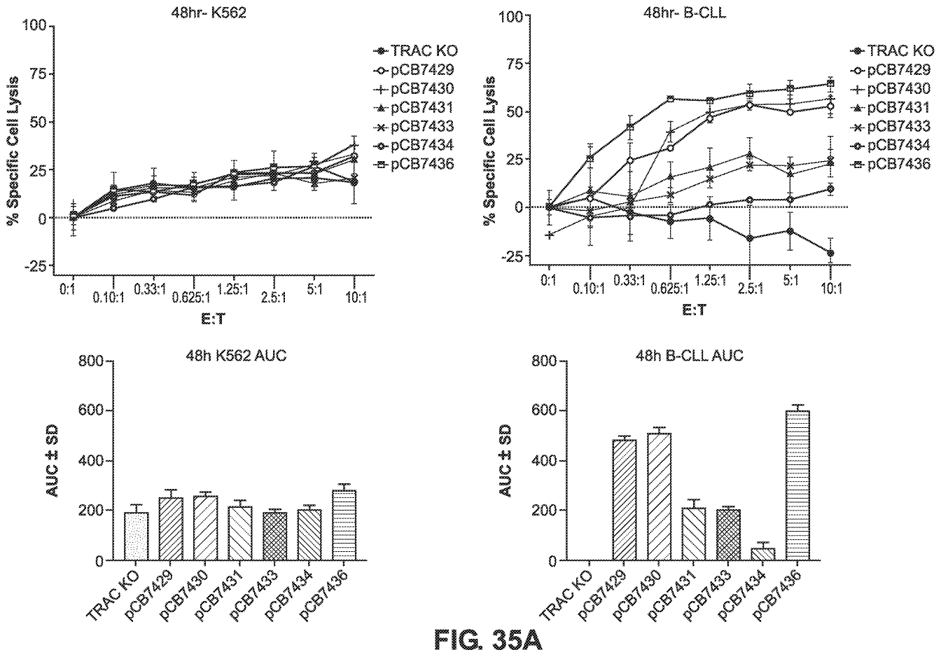

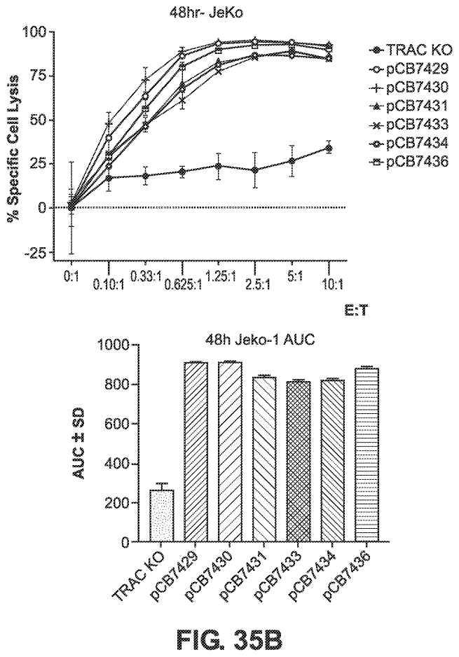

Area under the curve (AUC) was calculated for each effector to represent overall specific lysis against each ROR1-expressing target. Results are shown in FIG. 34 (pCB7432, pCB7437 or pCB7438 with JeKo-1 or with B-CLL), FIG. 35A (pCB7429, pCB7430, pCB7431, pCB7433, pCB7434 or pCB7436 with K562 or with B-CLL), and FIG. 35B (pCB7429, pCB7430, pCB7431, pCB7433, pCB7434 or pCB7436 with JeKo-1).

### Example 23. Cytokine Secretion by Alternative Anti-ROR1 CAR-Design CAR-T Cells in the Presence of ROR1-Expressing Targets

In this example, the CAR-T cells of Example 21 were assessed for cytokine secretion in the presence of ROR1-expressing target cells. TRAC KO T cells were used as a control. Cocultures with ROR1-negative cell line K562 was used as a control. In addition, effector-only cultures were used to measure background cytokine secretion.

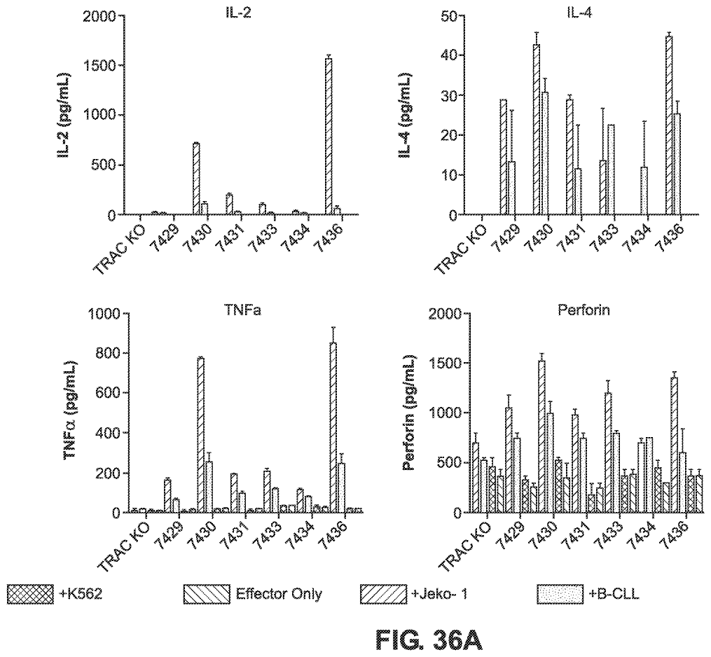

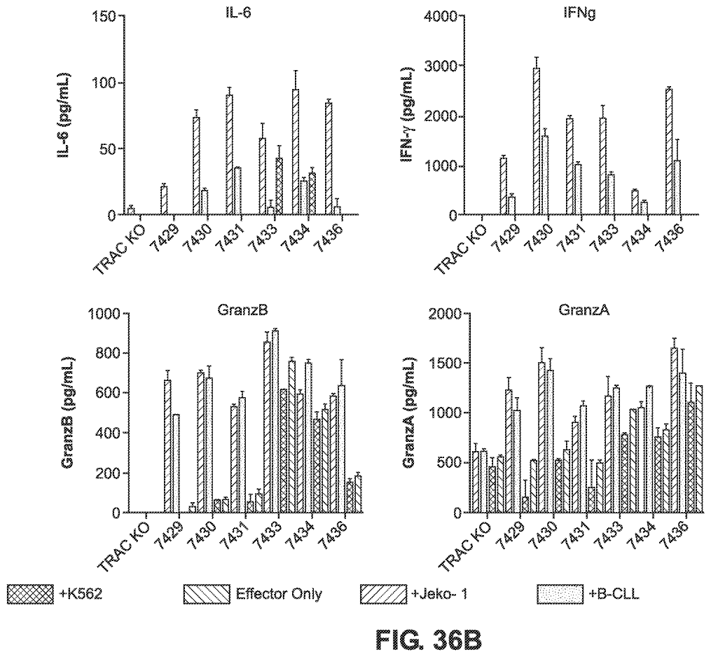

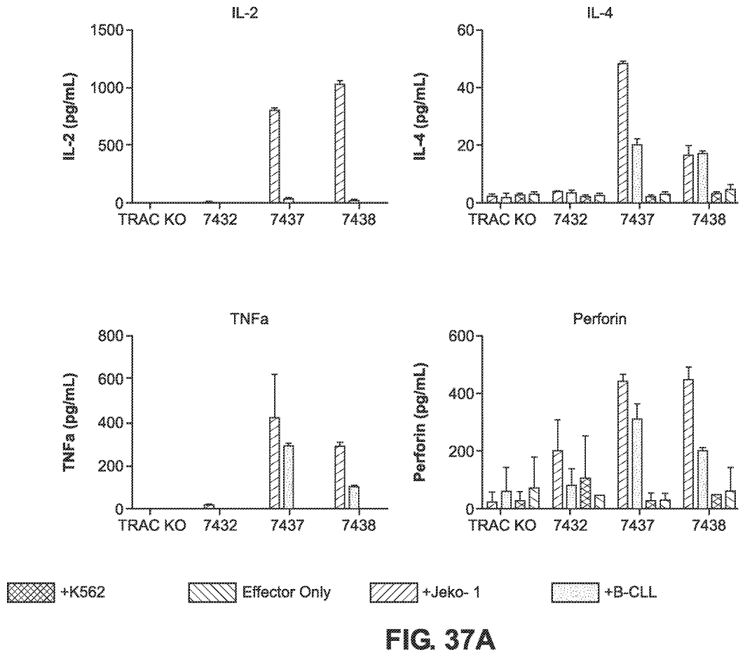

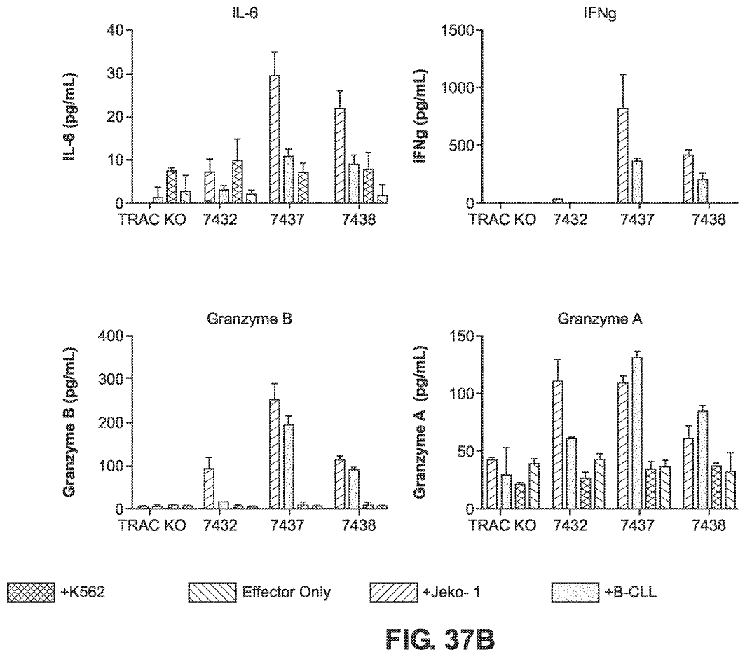

Effectors and target cells were cocultured at a 1:1 ratio for 24 hrs. Supernatant was collected and analyzed for presence of cytokines IFNγ, TNFα, IL-2, IL-4, IL-6, Perforin, Granzyme A and Granzyme B using the ProcartaPlex™ immunoassay kit and manufacturer-provided protocols (Life Technologies, Waltham, Mass.). Results are shown in FIG. 36A (pCB7429, pCB7430, pCB7431, pCB7433, pCB7434 or pCB7436 secreting IL-2, IL-4, TNFα or perform), FIG. 36B (pCB7429, pCB7430, pCB7431, pCB7433, pCB7434 or pCB7436 secreting IL-6, IFNγ, Granzyme A or Granzyme B), FIG. 37A (pCB7432, pCB7437 or pCB7438 secreting IL-2, IL-4, TNFα or perforin) and FIG. 37B (pCB7432, pCB7437 or pCB7438 secreting IL-6, IFNγ, Granzyme A or Granzyme B).

### Example 24. IL-36γ Secretion by Alternative Anti-ROR1 CAR-Design CAR-T Cells

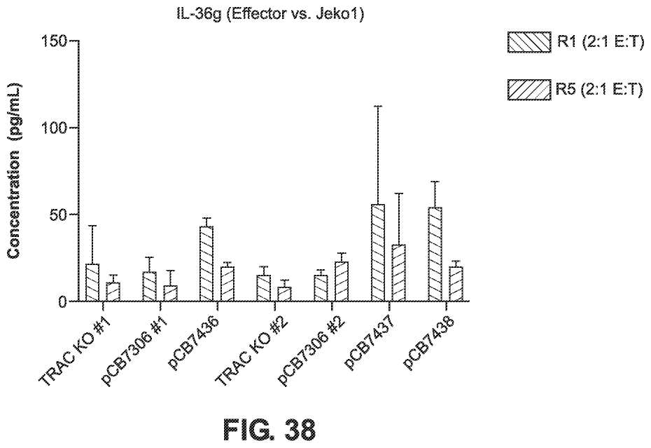

In this example, IL-36γ secretion by anti-ROR1 CAR-T cells (pCB7306 (FIG. 26), pCB7436, pCB7437, and pCB7438, Example 21, Table 3) and negative T cell control was assessed using the IL-36γ ELISA kit from R & D Systems (cat #DY2320-05) (Minneapolis, Minn.) according to manufacturer's instructions. Target JeKo-1 cells, ROR1-positive and engineered to express luciferase, were co-cultured with effector cells at increasing E:T ratios in the range of 0:1 to 2:1. Supernatant of the 2:1 E:T ratio was collected after each round before assessing cytotoxicity (see Example 22). Supernatant from round 1 and round 5 was assayed for IL-36γ concentration. Results are shown in FIG. 38.

### Example 25. In Vivo Anti-Tumor Activity of Alternative Anti-ROR1 CAR-Design CAR-T Cells

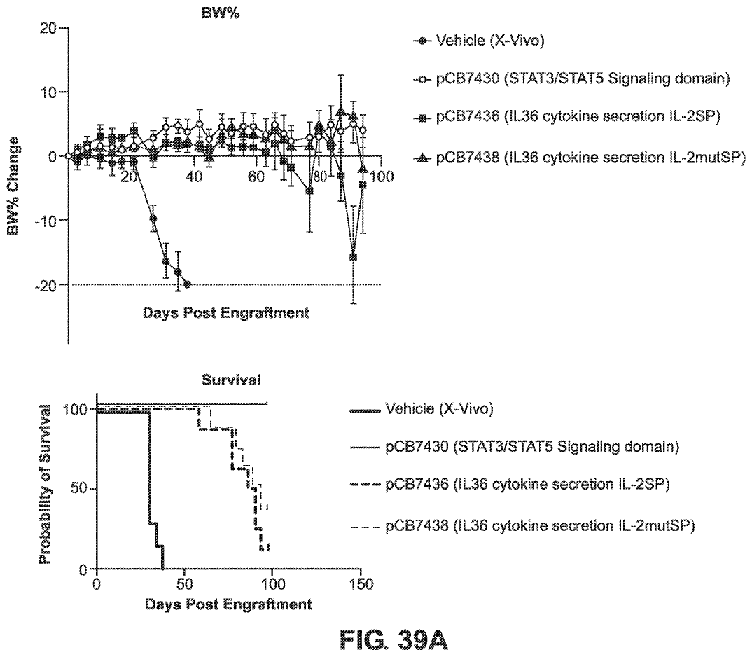

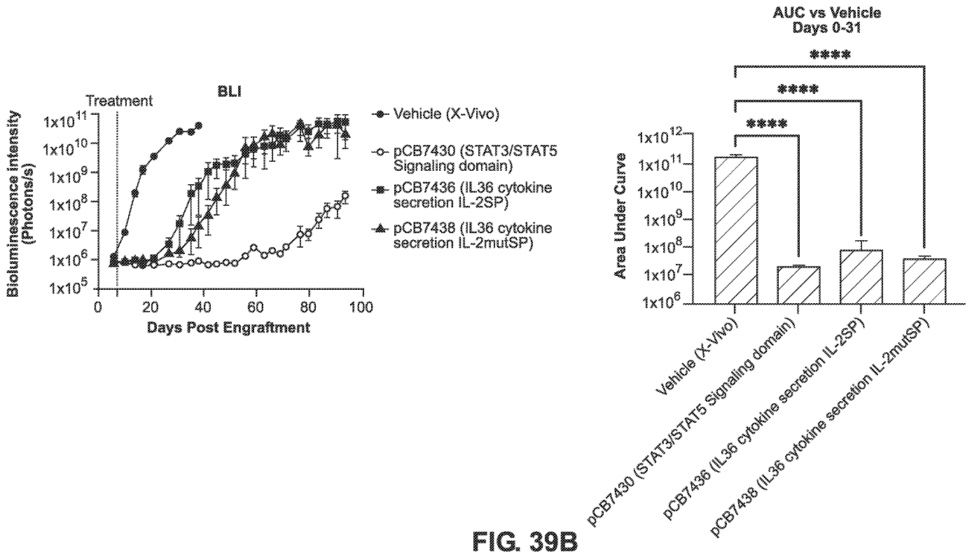

5×10⁵ JeKo-1-GFPluc tumor cells were injected intravenously (IV) into NGS female mice. At day 7 post-engraftment, CAR-T cells (Example 21) were also IV-injected at 1×10⁷ CAR⁺ T cells. Body weight and IVIS measurements are taken twice per week and mice are evaluated for anti-tumor activity by tracking body weight changes, survival and changes in tumor burden measured as bioluminescence intensity. Results are shown in FIG. 39A (body weight and probability of survival) and FIG. 39B (bioluminescence in photons and as AUC).

### Example 26. Comparison of the New Anti-ROR1 CAR-T Cells to Benchmark Anti-ROR1 CAR-T Cells

In this example, the CAR construct shown in FIG. 26 (pCB7306) was compared to the same CAR except the scFv was exchanged for the R12 scFv from the publication Hudecek et al., (2013) Receptor affinity and extracellular domain modifications affect tumor recognition by ROR1-specific chimeric antigen receptor T-cells, Clin. Cancer Res. 19:3153. The CAR-T cells expressing one of the two CARs were prepared as described in Example 13.

The resulting CAR-T cells expressing scFv PMC862 (instant invention) or scFv R12 (Hudecek et al., supra) were tested in parallel to compare in vitro and in vivo anti-tumor properties.

In Vitro Serial Rechallenge with JeKo1 Tumor Cells

The CAR-T cells “PMC862” and “R12” along with a TRAC KO control T cells were thawed and recovered in IL-2 containing media 24 hours prior to the first stimulation. Pre-stimulation (round zero, R0) CAR expression was evaluated using PE-conjugated human recombinant ROR1 protein. Comparable surface CAR expression was observed between PMC862 and R12 CAR-T cells.

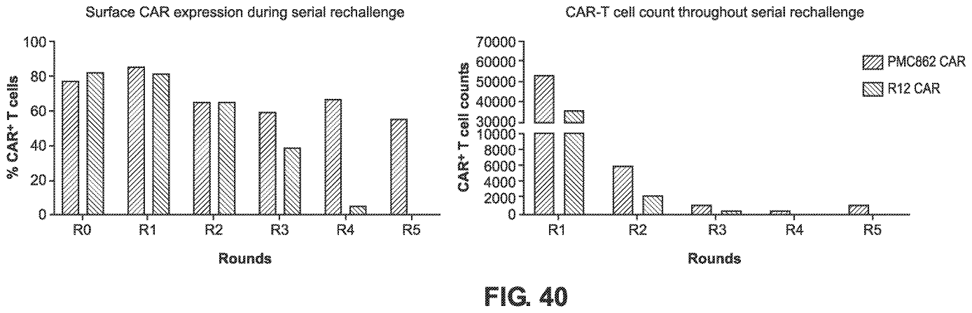

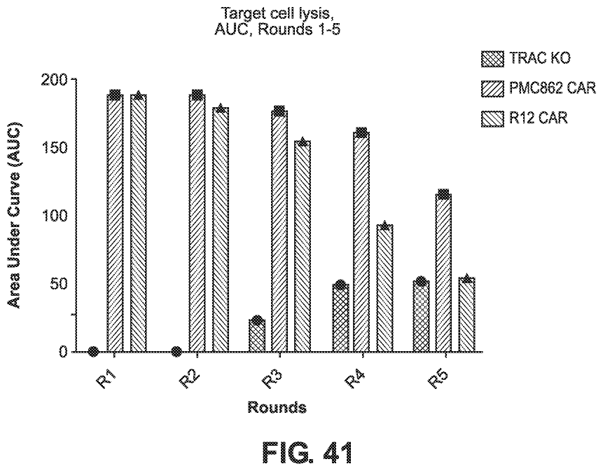

The T cells were then plated at different effector:target (E:T) ratios with the Jeko1-GFP:ffLuc target cells. Every 72-96 hours, specific lysis of target cells was quantified via luciferase-based luminescence readings. CAR-T cell phenotyping via flow cytometry was also conducted. CAR⁺ T cell counts were estimated using BD Countbright™ Absolute Counting Beads. Following analyte measurement at each round, the remaining CAR-T cells were restimulated with fresh JeKo1-GFP:ffLuc target cells. Serial rechallenge was continued until loss of specific lysis was observed for one or both anti-ROR1 CAR-T cells. Results are shown in FIG. 40 and FIG. 41.

The PMC862 CAR-T cells retained high CAR expression through 5 rounds of rechallenge while the benchmark R12 CAR-T cells started to lose CAR expression at round 3 with the expression becoming undetectable at round 5. This loss of R12 CAR-T cells correlates with a complete loss of specific lysis at round 5 (FIG. 40). The loss of CAR expression in R12 CAR-T cells correlated with loss of antigen-specific target cell lysis (FIG. 41).

### In Vivo Antitumor Activity in NGS Mice.

JeKo-1-GFP:ffLuc target cells in PBS were engrafted intravenously at 5×10⁵ cells/animal. Seven days post-engraftment, PMC862 and R12 anti-ROR1 CAR-T cells in X-Vivo serum-free medium were dosed intravenously at 10⁷ CAR⁺ T cells/animal. A vehicle only (X-Vivo medium) injection was used as a negative control. The CAR-positivity rates for the PMC862 and R12 CAR-T cells were 89.6% and 88.4% respectively.

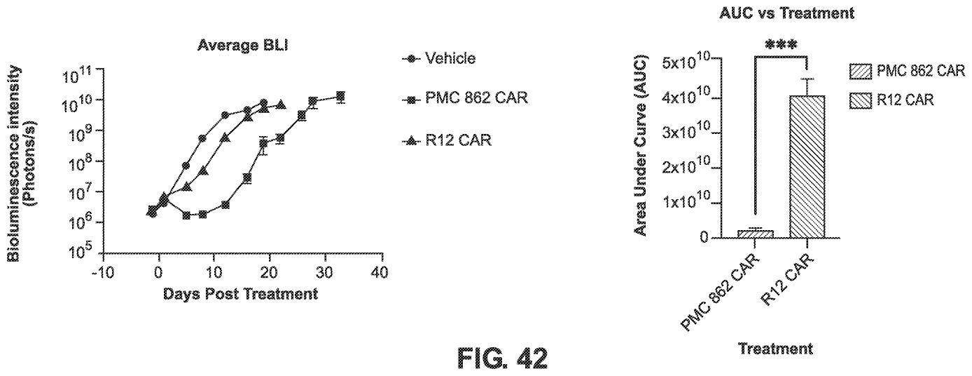

Starting at day 5 after CAR-T infusion, bioluminescence imaging (BLI) of animals was conducted 2×/week using the IVIS® Spectrum in vivo Imaging System. Individual BLI measurements (n=8 mice/group) were used to calculate average BLI and plot area under the curve (AUC) for day 0 through day 22 (FIG. 42). A Mann-Whitney test comparing the AUC of PMC862 (the CB-013 CAR) and the AUC of the R12 CAR indicate superior tumor control of the PMC862 CAR-T cells (*** indicates p<0.001).

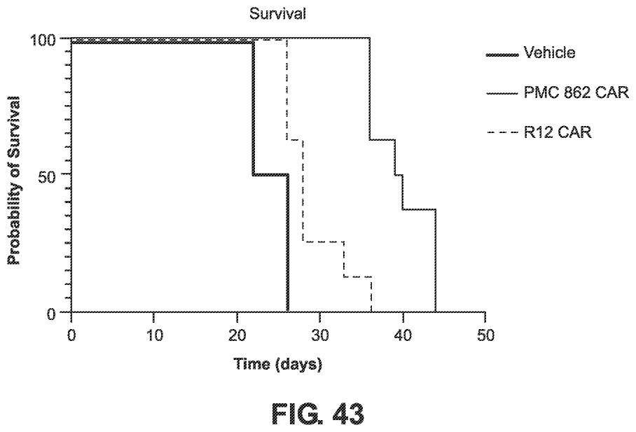

Animals were euthanized after losing >20% of their body weight compared to day 0 or after developing paralysis. Kaplan-Meier survival curves at any time (Mantel-Cox log-rank test) were developed (FIG. 43). The median survival of vehicle-treated group was 24 days, the median survival of the R12 CAR-T-treated group was 28 days, and the median survival of the PMC862 CAR-T-treated group was 39.5 days.

### Example 27. In Vivo Antitumor Activity of Anti-ROR1 CAR-iNK Cells with Multiple Genome Modifications

The in vivo experiment was designed essentially as Example 25. 3×10⁵ SKOV3-GFPluc tumor cells were injected intraperitoneally (IP) into NGS female mice. At day 7 post-engraftment, 2×10⁷ CAR-iNK cells were also IP-injected.

The CAR-iNK cells contained the CAR from pCB7306 (FIG. 26), a disrupted CBLB gene (Example 8), and a B2M-HLA-E fusion peptide in the B2M locus (Example 10). The CAR-iNK cells were also expressing an IL15 receptor fusion (Example 11). Unmodified iNK cells were used as a control.

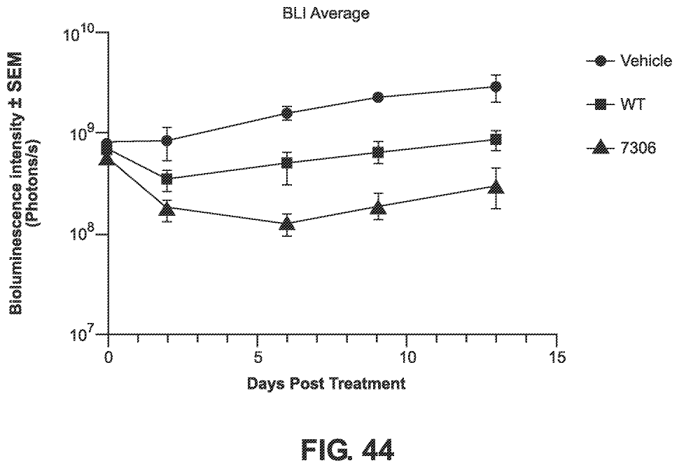

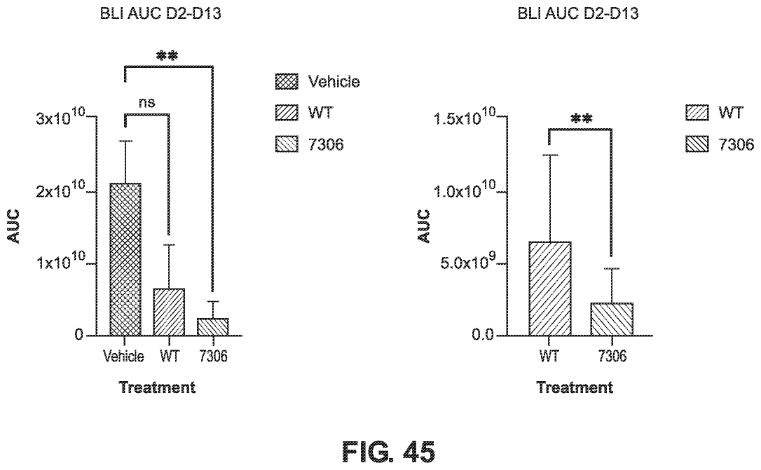

IVIS measurements of the mice were taken during days 2-13 post-treatment and changes in tumor burden were assessed from bioluminescence intensity. Results are shown in FIG. 44 (average bioluminescence, BLI) and FIG. 45 (area under the curve, AUC, ** indicates p<0.01).

### Example 28. Serial Rechallenge of Anti-ROR iNK Cells with Tumor Cells

The experiment was designed essentially as in Example 20. The iNK cells were subjected to 6 successive challenges by tumor cells. Two CAR-iNK cells were tested: pCB7306 expressed the CAR shown in FIG. 26, and pCB7447 expressed an identical CAR, except for an additional CD27 co-stimulatory domain inserted between the CD3zeta domain and the 4-1BB domain (referring to FIG. 26). Both types of CAR-iNK cells included a disrupted CBLB gene, a B2M-HLA-E fusion peptide in the B2M locus and expression of the IL15 receptor fusion (Example 27). The control iNK cells included a disrupted TRAC gene and like the CAR-iNK cells, also included a disrupted CBLB gene, a B2M-HLA-E fusion peptide in the B2M locus and expression of an IL15 receptor fusion described in Example 27, except the control iNK cells lacked a CAR. Unmodified iNK cells and iNK cells expressing the IL15 receptor fusion were used as two additional controls.

Two types of target cells were used: SKOV3, an ovarian tumor cell line with low ROR1 antigen density, and Hs764t, a gastric carcinoma cell line with high ROR1 antigen density.

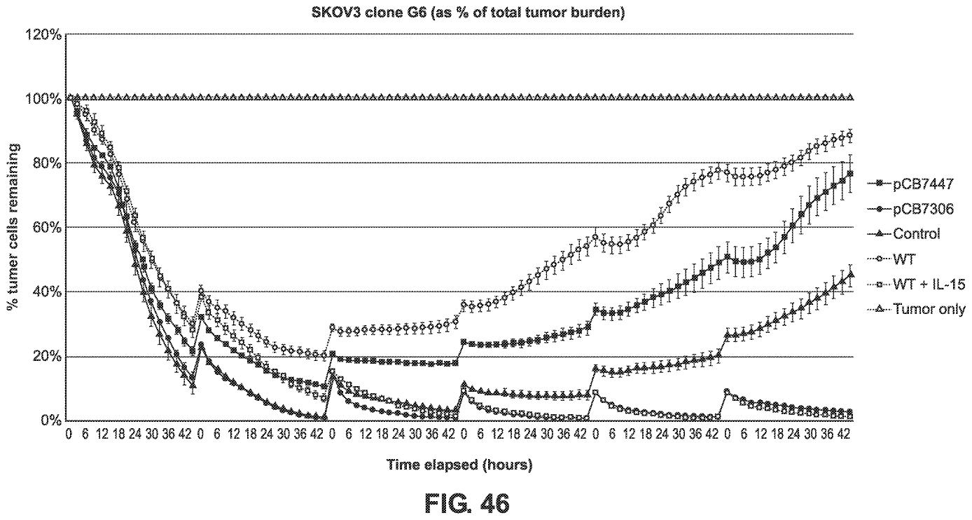

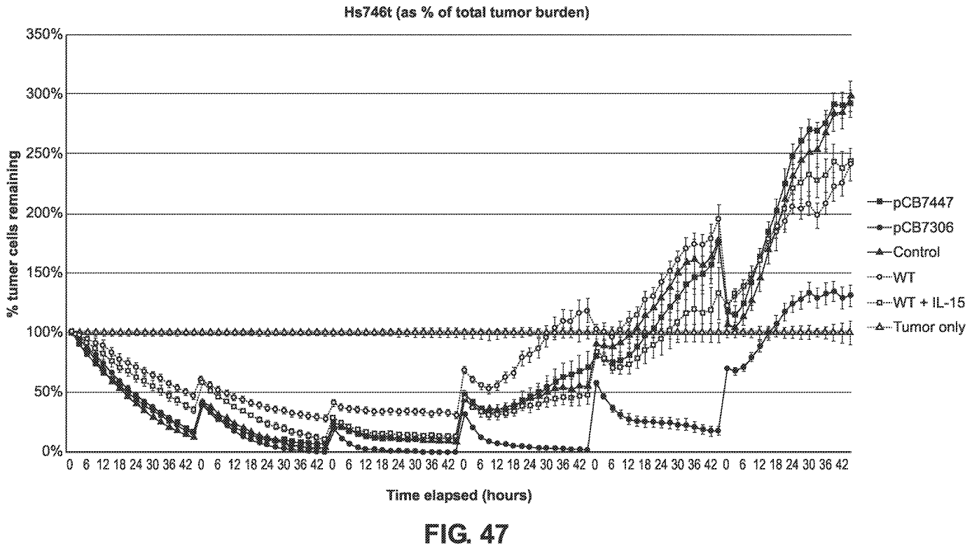

Target cells engineered to express luciferase were added to the effector cells at 1:1 E:T ratio. Every 42 hours 5,000 fresh target cells were added to each culture 5 additional times (6 challenges total). The proportion of remaining live tumor cells was assessed by a luminescence readout. Results are shown in FIG. 46 (SKOV3 targets) and FIG. 47 (Hs746t targets).

### Example 29. Multi-Dose of Anti-ROR1 CAR-iNK Cells with Multiple Genome Modifications

This in vivo experiment was designed essentially as Example 27. The two CAR-iNK designs were the same as in Example 28: the first CAR-iNKs having the CAR design pCB7306 and the second CAR-iNKs having the CAR design pCB7447, both sets of iNKs having the disrupted CBLB gene, the B2M-HLA-E fusion peptide in the B2M locus and expressing the IL15 receptor fusion (see Example 28). Unedited (“WT”) iNKs were used as a control.

Two types of target tumor cell lines were used: SKOV3, an ovarian tumor cell line with low ROR1 antigen density, and Hs764t, a gastric carcinoma cell line with high ROR1 antigen density.

3×10⁵ SKOV3-GFPluc or 10⁵ Hs746t-GFPluc tumor cells were injected intraperitoneally (IP) into NSG female mice (8 mice in each treatment group, except 7 mice in Hs746t pCB7447 and 6 mice in Hs746t pCB7306 treatment groups). At day 7 post-engraftment, 2×10⁷ CAR-iNK cells were also IP-injected. The second injection of 2×10⁷ CAR-iNKs was administered on day 8 post tumor cell engraftment and the third injection of 2×10⁷ CAR-iNKs was administered on day 11 post tumor cell engraftment.

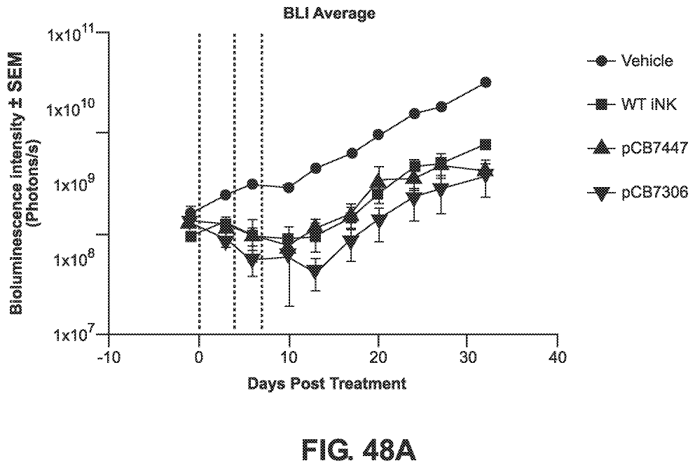

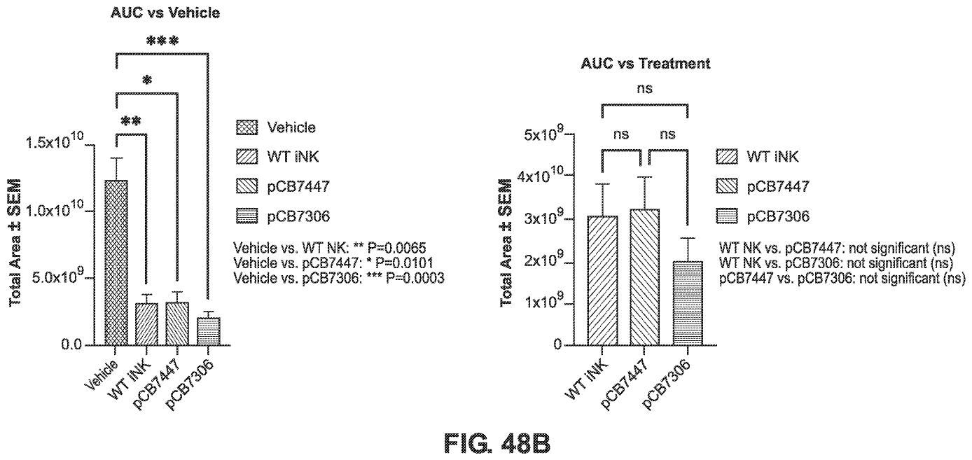

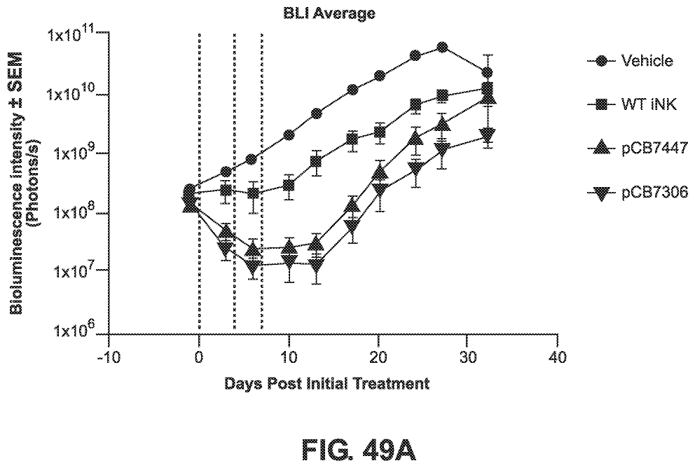

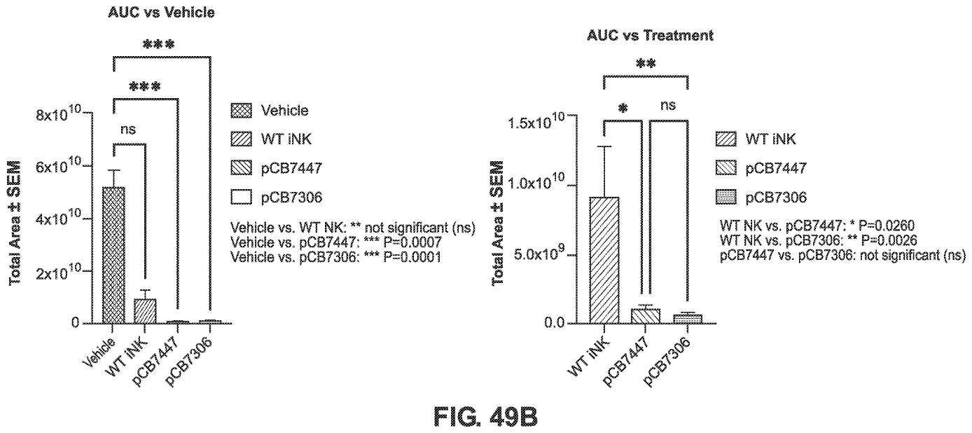

IVIS measurements of the mice were taken twice weekly after the initial iNK treatment, and changes in tumor burden were assessed from bioluminescence intensity. Results are shown in FIG. 48A (SKOV3 average bioluminescence, BLI), FIG. 48B (SKOV3 area under the curve, AUC calculated from bioluminescence), FIG. 49A (Hs746t average bioluminescence, BLI), FIG. 49B (Hs746t area under the curve, AUC) and FIG. 45 (area under the curve, AUC calculated from bioluminescence).

While the invention has been described in detail with reference to specific examples, it will be apparent to one skilled in the art that various modifications can be made within the scope of this invention. Thus, the scope of the invention should not be limited by the examples described herein, but by the claims presented below.

### Informal Sequence Listing

### Informal Sequence Table

| SEQ ID NO | Description | Type |
|---|---|---|
| SEQ ID NO: 1 | Humanized scFv (857) | AA |
| SEQ ID NO: 2 | Humanized VH (857) | AA |
| SEQ ID NO: 3 | Humanized VL (857) | AA |
| SEQ ID NO: 4 | Humanized CAR (857) | AA |
| SEQ ID NO: 5 | Humanized scFv (858) | AA |
| SEQ ID NO: 6 | Humanized VH (858) | AA |
| SEQ ID NO: 7 | Humanized VL (858) | AA |
| SEQ ID NO: 8 | Humanized CAR (858) | AA |
| SEQ ID NO: 9 | Humanized scFv (862) | AA |
| SEQ ID NO: 10 | Humanized VH (862) | AA |
| SEQ ID NO: 11 | Humanized VL (862) | AA |
| SEQ ID NO: 12 | Humanized CAR (862) | AA |
| SEQ ID NO: 13 | 857 scFv | NA |
| SEQ ID NO: 14 | 857 CAR | NA |
| SEQ ID NO: 15 | 858 scFv | NA |
| SEQ ID NO: 16 | 858 CAR | NA |
| SEQ ID NO: 17 | 862 scFv | NA |
| SEQ ID NO: 18 | 858 CAR | NA |
| SEQ ID NO: 19 | CD8 hinge | NA |
| SEQ ID NO: 20 | CD8 hinge | AA |
| SEQ ID NO: 21 | CD8 TM | NA |
| SEQ ID NO: 22 | CD8 TM | AA |
| SEQ ID NO: 23 | CD3 zeta | NA |
| SEQ ID NO: 24 | CD3 zeta | AA |
| SEQ ID NO: 25 | 4-1BB | NA |
| SEQ ID NO: 26 | 4-1BB | AA |
| SEQ ID NO: 27 | Short linker scFv | AA |
| SEQ ID NO: 28 | Short linker scFv | NA |
| SEQ ID NO: 29 | CDR 1 (heavy chain) | AA |
| SEQ ID NO: 30 | CDR 2 (heavy chain) | AA |
| SEQ ID NO: 31 | CDR 3 (heavy chain) | AA |
| SEQ ID NO: 32 | CDR 1 (light chain) | AA |
| SEQ ID NO: 33 | CDR 2 (light chain) | AA |
| SEQ ID NO: 34 | CDR 3 (light chain) | AA |
| SEQ ID NO: 35 | CAR (long linker scFv) pCB7306 | AA |
| SEQ ID NO: 36 | CAR (long linker scFv) | NA |
| SEQ ID NO: 37 | CAR (short linker scFv) pCB7339 | AA |
| SEQ ID NO: 38 | CAR (short linker scFv) | NA |
| SEQ ID NO: 39 | STAT3 motif YXXQ | AA |
| SEQ ID NO: 40 | STAT3 motif YRHQ | AA |
| SEQ ID NO: 41 | STAT5 motif YXXL | AA |
| SEQ ID NO: 42 | STAT5 motif YLSL | AA |
| SEQ ID NO: 43 | JAK motif LKCNTPDPS | AA |
| SEQ ID NO: 44 | IL36γ -IL2SP | AA |
| SEQ ID NO: 45 | IL36γ --IL2SP | NA |
| SEQ ID NO: 46 | IL36γ-IL36SP | AA |
| SEQ ID NO: 47 | IL36γ-IL36SP | NA |
| SEQ ID NO: 48 | IL36γ-IL2 mutant SP | AA |
| SEQ ID NO: 49 | IL36γ-IL2 mutant SP | NA |
| SEQ ID NO: 50 | IL-2Rb | AA |
| SEQ ID NO: 51 | IL-2Rb | NA |

### Informal Sequence Listing

| SEQ ID NO: 1 | Humanized scFv (857) AA |
|---|---|
| EVQLVESGGGLIQPGGSLRLSCAASGFTFSTYAMSWVRQAPGKGLEWVSSISSGGNTY | |
| YADSVKGRFTISRDNSKNTLYLQMNSLRAEDTAVYYCARDSYYFGNSVYYAMDYW | |
| GAGTTVTVSSGGGGSGGGGSGGGGSDIQMTQSPSSLSASVGDRVTITCKASQDINSYF | |
| SWFQQKPGKAPKSLIYRANRLVSGVPSRFSGSGSGTDFTLTISSLQPEDFATYYCLQYD | |
| EFPYTFGGGTRLEIK | |
| SEQ ID NO: 2 | Humanized VH (857) AA |
| EVQLVESGGGLIQPGGSLRLSCAASGFTFSTYAMSWVRQAPGKGLEWVSSISSGGNTY | |
| YADSVKGRFTISRDNSKNTLYLQMNSLRAEDTAVYYCARDSYYFGNSVYYAMDYW | |
| GAGTTVTV | |
| SEQ ID NO: 3 | Humanized VL (857) AA |
| DIQMTQSPSSLSASVGDRVTITCKASQDINSYFSWFQQKPGKAPKSLIYRANRLVSGVP | |
| SRFSGSGSGTDFTLTISSLQPEDFATYYCLQYDEFPYTFGGGTRLEIK | |
| SEQ ID NO: 4 | Humanized CAR (857) AA |
| MALPVTALLLPLALLLHAARPASEVQLVESGGGLIQPGGSLRLSCAASGFTFSTYAMS | |
| WVRQAPGKGLEWVSSISSGGNTYYADSVKGRFTISRDNSKNTLYLQMNSLRAEDTAV | |
| YYCARDSYYFGNSVYYAMDYWGAGTTVTVSSGGGGSGGGGSGGGGSDIQMTQSPS | |
| SLSASVGDRVTITCKASQDINSYFSWFQQKPGKAPKSLIYRANRLVSGVPSRFSGSGSG | |
| TDFTLTISSLQPEDFATYYCLQYDEFPYTFGGGTRLEIKLEKPTTTPAPRPPTPAPTIASQ | |
| PLSLRPEASRPAAGGAVHTRGLDFASDKPFWVLVVVGGVLACYSLLVTVAFIIFWVK | |
| RGRKKLLYIFKQPFMRPVQTTQEEDGCSCRFPEEEEGGCELRVKFSRSADAPAYQQGQ | |
| NQLYNELNLGRREEYDVLDKRRGRDPEMGGKPQRRKNPQEGLYNELQKDKMAEAY | |
| SEIGMKGERRRGKGHDGLYQGLSTATKDTYDALHMQALPPR | |
| SEQ ID NO: 5 | Humanized scFv (858) AA |
| QVQLVESGGGVVQPGRSLRLSCAASGFTFSTYAMSWVRQAPGKGLEWVASISSGGNT | |
| YYPDSVKGRFTISRDNSKNTLYLQMNSLRAEDTAVYYCARDSYYFGNSVYYAMDY | |
| WGAGTTVTVSSGGGGSGGGGSGGGGSDIQMTQSPSSLSASVGDRVTITCKASQDINS | |
| YFSWFQQKPGKAPKSLIYRANRLVSGVPSRFSGSGSGTDFTLTISSLQPEDFATYYCLQ | |
| YDEFPYTFGGGTRLEIK | |
| SEQ ID NO: 6 | Humanized VH (858) AA |
| QVQLVESGGGVVQPGRSLRLSCAASGFTFSTYAMSWVRQAPGKGLEWVASISSGGNT | |
| YYPDSVKGRFTISRDNSKNTLYLQMNSLRAEDTAVYYCARDSYYFGNSVYYAMDY | |
| WGAGTTVTVSS | |
| SEQ ID NO: 7 | Humanized VL (858) AA |
| DIQMTQSPSSLSASVGDRVTITCKASQDINSYFSWFQQKPGKAPKSLIYRANRLVSGVP | |
| SRFSGSGSGTDFTLTISSLQPEDFATYYCLQYDEFPYTFGGGTRLEIK | |
| SEQ ID NO: 8 | Humanized CAR (858) AA |
| MALPVTALLLPLALLLHAARPASQVQLVESGGGVVQPGRSLRLSCAASGFTFSTYAM | |
| SWVRQAPGKGLEWVASISSGGNTYYPDSVKGRFTISRDNSKNTLYLQMNSLRAEDTA | |
| VYYCARDSYYFGNSVYYAMDYWGAGTTVTVSSGGGGSGGGGSGGGGSDIQMTQSP | |
| SSLSASVGDRVTITCKASQDINSYFSWFQQKPGKAPKSLIYRANRLVSGVPSRFSGSGS | |
| GTDFTLTISSLQPEDFATYYCLQYDEFPYTFGGGTRLEIKLEKPTTTPAPRPPTPAPTIAS | |
| QPLSLRPEASRPAAGGAVHTRGLDFASDKPFWVLVVVGGVLACYSLLVTVAFIIFWV | |
| KRGRKKLLYIFKQPFMRPVQTTQEEDGCSCRFPEEEEGGCELRVKFSRSADAPAYQQG | |
| QNQLYNELNLGRREEYDVLDKRRGRDPEMGGKPQRRKNPQEGLYNELQKDKMAEA | |
| YSEIGMKGERRRGKGHDGLYQGLSTATKDTYDALHMQALPPR | |
| SEQ ID NO: 9 | Humanized scFv (862) AA |
| QVQLVESGGGVVQPGRSLRLSCAASGFTFSTYAMSWVRQAPGKGLEWVASISSGGNT | |
| YYPDSVKGRFTISRDNSKNTLYLQMNSLRAEDTAVYYCARDSYYFGNSVYYAMDY | |
| WGAGTTVTVSSGGGGSGGGGSGGGGSDIQMTQSPSSLSASVGDRVTITCKASQDINS | |
| YFSWYQQKPGKAPKLLIYRANRLVTGVPSRFSGSGSGTDFTFTISSLQPEDIATYYCLQ | |
| YDEFPYTFGGGTRLEIK | |
| SEQ ID NO: 10 | Humanized VH (862) AA |
| QVQLVESGGGVVQPGRSLRLSCAASGFTFSTYAMSWVRQAPGKGLEWVASISSGGNT | |
| YYPDSVKGRFTISRDNSKNTLYLQMNSLRAEDTAVYYCARDSYYFGNSVYYAMDY | |
| WGAGTTVTVSS | |
| SEQ ID NO: 11 | Humanized VL (862) AA |
| DIQMTQSPSSLSASVGDRVTITCKASQDINSYFSWYQQKPGKAPKLLIYRANRLVTGV | |
| PSRFSGSGSGTDFTFTISSLQPEDIATYYCLQYDEFPYTFGGGTRLEIK | |
| SEQ ID NO: 12 | Humanized CAR (862) AA |
| MALPVTALLLPLALLLHAARPASQVQLVESGGGVVQPGRSLRLSCAASGFTFSTYAM | |
| SWVRQAPGKGLEWVASISSGGNTYYPDSVKGRFTISRDNSKNTLYLQMNSLRAEDTA | |
| VYYCARDSYYFGNSVYYAMDYWGAGTTVTVSSGGGGSGGGGSGGGGSDIQMTQSP | |
| SSLSASVGDRVTITCKASQDINSYFSWYQQKPGKAPKLLIYRANRLVTGVPSRFSGSGS | |
| GTDFTFTISSLQPEDIATYYCLQYDEFPYTFGGGTRLEIKLEKPTTTPAPRPPTPAPTIAS | |
| QPLSLRPEASRPAAGGAVHTRGLDFASDKPFWVLVVVGGVLACYSLLVTVAFIIFWV | |
| KRGRKKLLYIFKQPFMRPVQTTQEEDGCSCRFPEEEEGGCELRVKFSRSADAPAYQQG | |
| QNQLYNELNLGRREEYDVLDKRRGRDPEMGGKPQRRKNPQEGLYNELQKDKMAEA | |
| YSEIGMKGERRRGKGHDGLYQGLSTATKDTYDALHMQALPPR | |
| SEQ ID NO: 13 | 857 scFv NA |
| GAAGTACAGCTTGTTGAATCAGGTGGTGGTCTTATTCAGCCAGGAGGCTCCTTGCG | |
| ACTGAGCTGTGCCGCTTCTGGGTTCACCTTTAGCACTTACGCAATGAGTTGGGTCC | |
| GACAAGCCCCAGGTAAGGGATTGGAATGGGTAAGTTCCATTTCCAGCGGAGGGAA | |
| CACTTATTACGCCGATTCTGTGAAAGGACGCTTTACTATATCCCGAGACAATAGTA | |
| AAAACACATTGTATTTGCAAATGAACTCTTTGAGGGCCGAGGACACTGCCGTCTA | |
| CTATTGTGCCCGCGACAGCTATTATTTCGGCAACTCTGTGTATTACGCGATGGATT | |
| ACTGGGGTGCCGGCACAACTGTCACCGTTTCATCTGGCGGAGGAGGCAGTGGCGG | |
| AGGGGGCTCAGGCGGTGGTGGAAGTGATATTCAAATGACCCAATCACCCTCTTCA | |
| TTGTCTGCAAGCGTAGGTGACCGAGTCACGATAACCTGCAAAGCCTCTCAAGATA | |
| TTAATTCATACTTTTCTTGGTTTCAACAAAAACCGGGAAAGGCGCCTAAGTCATTG | |
| ATTTACCGCGCGAACCGGTTGGTATCAGGAGTACCGTCAAGATTCTCAGGGAGTG | |
| GGTCAGGCACAGATTTCACACTCACTATTTCTTCCTTGCAACCTGAAGACTTCGCA | |
| ACCTATTATTGCTTGCAGTATGATGAGTTTCCGTACACTTTCGGGGGGGGTACAAG | |
| GCTGGAGATCAAA | |
| SEQ ID NO: 14 | 857 CAR NA |
| ATGGCCTTACCAGTGACCGCCTTGCTCCTGCCGCTGGCCTTGCTGCTCCACGCCGC | |
| CAGGCCGGCTAGCGAAGTACAGCTTGTTGAATCAGGTGGTGGTCTTATTCAGCCA | |
| GGAGGCTCCTTGCGACTGAGCTGTGCCGCTTCTGGGTTCACCTTTAGCACTTACGC | |
| AATGAGTTGGGTCCGACAAGCCCCAGGTAAGGGATTGGAATGGGTAAGTTCCATT | |
| TCCAGCGGAGGGAACACTTATTACGCCGATTCTGTGAAAGGACGCTTTACTATATC | |
| CCGAGACAATAGTAAAAACACATTGTATTTGCAAATGAACTCTTTGAGGGCCGAG | |
| GACACTGCCGTCTACTATTGTGCCCGCGACAGCTATTATTTCGGCAACTCTGTGTA | |
| TTACGCGATGGATTACTGGGGTGCCGGCACAACTGTCACCGTTTCATCTGGCGGA | |
| GGAGGCAGTGGCGGAGGGGGCTCAGGCGGTGGTGGAAGTGATATTCAAATGACC | |
| CAATCACCCTCTTCATTGTCTGCAAGCGTAGGTGACCGAGTCACGATAACCTGCAA | |
| AGCCTCTCAAGATATTAATTCATACTTTTCTTGGTTTCAACAAAAACCGGGAAAGG | |
| CGCCTAAGTCATTGATTTACCGCGCGAACCGGTTGGTATCAGGAGTACCGTCAAG | |
| ATTCTCAGGGAGTGGGTCAGGCACAGATTTCACACTCACTATTTCTTCCTTGCAAC | |
| CTGAAGACTTCGCAACCTATTATTGCTTGCAGTATGATGAGTTTCCGTACACTTTC | |
| GGGGGGGGTACAAGGCTGGAGATCAAACTCGAGAAGCCCACCACGACGCCAGCG | |
| CCGCGACCACCAACACCGGCGCCCACCATCGCGTCGCAGCCCCTGTCCCTGCGCC | |
| CAGAGGCGAGCCGGCCAGCGGCGGGGGGCGCAGTGCACACGAGGGGGCTGGACT | |
| TCGCCAGTGATAAGCCCTTTTGGGTGCTGGTGGTGGTTGGTGGAGTCCTGGCTTGC | |
| TATAGCTTGCTAGTAACAGTGGCCTTTATTATTTTCTGGGTGAAACGGGGCAGAAA | |
| GAAACTCCTGTATATATTCAAACAACCATTTATGAGACCAGTACAAACTACTCAA | |
| GAGGAAGATGGCTGTAGCTGCCGATTTCCAGAAGAAGAAGAAGGAGGATGTGAA | |
| CTGAGAGTGAAGTTCAGCAGGAGCGCAGACGCCCCCGCGTACCAGCAGGGCCAG | |
| AACCAGCTCTATAACGAGCTCAATCTAGGACGAAGAGAGGAGTACGATGTTTTGG | |
| ACAAGAGACGTGGCCGGGACCCTGAGATGGGGGGAAAGCCGCAGAGAAGGAAG | |
| AACCCTCAGGAAGGCCTCTACAATGAACTGCAGAAAGATAAGATGGCGGAGGCC | |
| TACAGTGAGATTGGGATGAAAGGCGAGCGCCGGAGGGGCAAGGGGCACGATGGC | |
| CTTTACCAGGGTCTCAGTACAGCCACCAAGGACACCTACGACGCCCTTCACATGC | |
| AGGCCCTGCCCCCTCGCTAA | |
| SEQ ID NO: 15 | 858 scFv NA |
| CAGGTACAATTGGTAGAGTCCGGCGGAGGGGTTGTTCAGCCAGGACGGTCTTGCG | |
| TTGTCTTGTGCTGCGTCAGGATTCACATTCTCAACGTACGCGATGTCTTGGGTGCG | |
| CCAAGCTCCCGGTAAAGGGCTGGAATGGGTGGCCTCAATCTCATCTGGAGGGAAC | |
| ACTTACTACCCTGATAGTGTTAAAGGTCGCTTTACTATCTCAAGGGACAATAGCAA | |
| GAATACCTTGTATCTGCAAATGAACTCACTTAGAGCAGAGGACACAGCGGTATAT | |
| TACTGTGCTAGAGACTCATATTATTTCGGCAACTCCGTTTATTACGCGATGGATTA | |
| CTGGGGCGCAGGGACTACGGTAACTGTATCTTCTGGTGGTGGAGGGTCTGGGGGC | |
| GGGGGTAGTGGCGGCGGTGGCAGTGACATCCAGATGACACAGTCTCCGTCTTCAT | |
| TGAGTGCAAGCGTCGGCGATCGGGTTACCATTACGTGTAAGGCAAGTCAGGACAT | |
| CAACAGTTATTTTTCATGGTTTCAACAAAAGCCTGGAAAAGCGCCGAAATCACTC | |
| ATTTACCGAGCTAATAGGCTTGTCTCTGGCGTTCCGTCTCGCTTCAGTGGAAGTGG | |
| GAGCGGTACTGATTTTACCCTCACCATATCAAGCCTCAACCGGAGGATTTTGCCC | |
| GTACTATTGTCTCCAGTACGATGAATTTCCATATACGTTTGGCGGCGGGACTCGCT | |
| TGGAGATTAAA | |
| SEQ ID NO: 16 | 858 CAR NA |
| ATGGCCTTACCAGTGACCGCCTTGCTCCTGCCGCTGGCCTTGCTGCTCCACGCCGC | |
| CAGGCCGGCTAGCCAGGTACAATTGGTAGAGTCCGGCGGAGGGGTTGTTCAGCCA | |
| GGACGGTCCTTGCGGTTGTCTTGTGCTGCGTCAGGATTCACATTCTCAACGTACGC | |
| GATGTCTTGGGTGCGCCAAGCTCCCGGTAAAGGGCTGGAATGGGTGGCCTCAATC | |
| TCATCTGGAGGGAACACTTACTACCCTGATAGTGTTAAAGGTCGCTTTACTATCTC | |
| AAGGGACAATAGCAAGAATACCTTGTATCTGCAAATGAACTCACTTAGAGCAGAG | |
| GACACAGCGGTATATTACTGTGCTAGAGACTCATATTATTTCGGCAACTCCGTTTA | |
| TTACGCGATGGATTACTGGGGCGCAGGGACTACGGTAACTGTATCTTCTGGTGGT | |
| GGAGGGTCTGGGGGCGGGGGTAGTGGCGGCGGTGGCAGTGACATCCAGATGACA | |
| CAGTCTCCGTCTTCATTGAGTGCAAGCGTCGGCGATCGGGTTACCATTACGTGTAA | |
| GGCAAGTCAGGACATCAACAGTTATTTTTCATGGTTTCAACAAAAGCCTGGAAAA | |
| GCGCCGAAATCACTCATTTACCGAGCTAATAGGCTTGTCTCTGGCGTTCCGTCTCG | |
| CTTCAGTGGAAGTGGGAGCGGTACTGATTTTACCCTCACCATATCAAGCCTTCAAC | |
| CGGAGGATTTTGCCACGTACTATTGTCTCCAGTACGATGAATTTCCATATACGTTT | |
| GGCGGCGGGACTCGCTTGGAGATTAAACTCGAGAAGCCCACCACGACGCCAGCGC | |
| CGCGACCACCAACACCGGCGCCCACCATCGCGTCGCAGCCCCTGTCCCTGCGCCC | |
| AGAGGCGAGCCGGCCAGCGGCGGGGGGCGCAGTGCACACGAGGGGGCTGGACTT | |
| CGCCAGTGATAAGCCCTTTTGGGTGCTGGTGGTGGTTGGTGGAGTCCTGGCTTGCT | |
| ATAGCTTGCTAGTAACAGTGGCCTTTATTATTTTCTGGGTGAAACGGGGCAGAAA | |
| GAAACTCCTGTATATATTCAAACAACCATTTATGAGACCAGTACAAACTACTCAA | |
| GAGGAAGATGGCTGTAGCTGCCGATTTCCAGAAGAAGAAGAAGGAGGATGTGAA | |
| CTGAGAGTGAAGTTCAGCAGGAGCGCAGACGCCCCCGCGTACCAGCAGGGCCAG | |
| AACCAGCTCTATAACGAGCTCAATCTAGGACGAAGAGAGGAGTACGATGTTTTGG | |
| ACAAGAGACGTGGCCGGGACCCTGAGATGGGGGGAAAGCCGCAGAGAAGGAAG | |
| AACCCTCAGGAAGGCCTCTACAATGAACTGCAGAAAGATAAGATGGCGGAGGCC | |
| TACAGTGAGATTGGGATGAAAGGCGAGCGCCGGAGGGGCAAGGGGCACGATGGC | |
| CTTTACCAGGGTCTCAGTACAGCCACCAAGGACACCTACGACGCCCTTCACATGC | |
| AGGCCCTGCCCCCTCGCTAA | |
| SEQ ID NO: 17 | 862 scFv NA |
| CAGGTACAACTGGTGGAATCCGGCGGGGGAGTAGTACAGCCCGGACGATCTCTTC | |
| GACTCTCATGTGCAGCGTCCGGGTTCACTTTTTCTACCTACGCAATGTCATGGGTA | |
| CGACAGGCGCCGGGCAAAGGCCTCGAATGGGTTGCATCCATTTCATCAGGAGGTA | |
| ATACATATTATCCTGATTCAGTCAAGGGCCGATTCACGATTAGTCGAGATAATAGC | |
| AAGAACACTCTCTACTTGCAGATGAACTCCCTGCGGGCTGAGGACACGGCCGTGT | |
| ATTATTGCGCTCGCGATAGTTATTACTTCGGCAATTCCGTATATTATGCGATGGAC | |
| TATTGGGGCGCCGGTACTACCGTGACTGTTTCCTCTGGTGGGGGTGGGTCCGGGG | |
| GCGGTGGTTCAGGTGGAGGCGGATCCGACATTCAAATGACCCAGTCTCCCTCAAG | |
| TTTGTCTGCATCTGTTGGCGATAGAGTTACAATAACATGCAAAGCCAGTCAAGAC | |
| ATCAACTCATACTTCTCCTGGTATCAACAAAAGCCAGGAAAAGCTCCGAAACTGT | |
| TGATCTACCGGGCCAACCGGCTGGTCACTGGCGTGCCATCCCGGTTCAGTGGCAG | |
| CGGAAGCGGAACAGATTTCACGTTTACCATCTCTAGCCTCCAACCGGAGGACATC | |
| GCAACATACTATTGCCTTCAGTATGATGAGTTTCCCTACACTTTCGGTGGCGGCAC | |
| CCGACTTGAGATCAAA | |
| SEQ ID NO: 18 | 862 CAR NA |
| ATGGCCTTACCAGTGACCGCCTTGCTCCTGCCGCTGGCCTTGCTGCTCCACGCCGC | |
| CAGGCCGGCTAGCCAGGTACAACTGGTGGAATCCGGCGGGGGAGTAGTACAGCC | |
| CGGACGATCTCTTCGACTCTCATGTGCAGCGTCCGGGTTCACTTTTTCTACCTACG | |
| CAATGTCATGGGTACGACAGGCGCCGGGCAAAGGCCTCGAATGGGTTGCATCCAT | |
| TTCATCAGGAGGTAATACATATTATCCTGATTCAGTCAAGGGCCGATTCACGATTA | |
| GTCGAGATAATAGCAAGAACACTCTCTACTTGCAGATGAACTCCCTGCGGGCTGA | |
| GGACACGGCCGTGTATTATTGCGCTCGCGATAGTTATTACTTCGGCAATTCCGTAT | |
| ATTATGCGATGGACTATTGGGGCGCCGGTACTACCGTGACTGTTTCCTCTGGTGGG | |
| GGTGGGTCCGGGGGCGGTGGTTCAGGTGGAGGCGGATCCGACATTCAAATGACCC | |
| AGTCTCCCTCAAGTTTGTCTGCATCTGTTGGCGATAGAGTTACAATAACATGCAAA | |
| GCCAGTCAAGACATCAACTCATACTTCTCCTGGTATCAACAAAAGCCAGGAAAAG | |
| CTCCGAAACTGTTGATCTACCGGGCCAACCGGCTGGTCACTGGCGTGCCATCCCG | |
| GTTCAGTGGCAGCGGAAGCGGAACAGATTTCACGTTTACCATCTCTAGCCTCCAA | |
| CCGGAGGACATCGCAACATACTATTGCCTTCAGTATGATGAGTTTCCCTACACTTT | |
| CGGTGGCGGCACCCGACTTGAGATCAAACTCGAGAAGCCCACCACGACGCCAGC | |
| GCCGCGACCACCAACACCGGCGCCCACCATCGCGTCGCAGCCCCTGTCCCTGCGC | |
| CCAGAGGCGAGCCGGCCAGCGGCGGGGGGCGCAGTGCACACGAGGGGGCTGGAC | |
| TTCGCCAGTGATAAGCCCTTTTGGGTGCTGGTGGTGGTTGGTGGAGTCCTGGCTTG | |
| CTATAGCTTGCTAGTAACAGTGGCCTTTATTATTTTCTGGGTGAAACGGGGCAGAA | |
| AGAAACTCCTGTATATATTCAAACAACCATTTATGAGACCAGTACAAACTACTCA | |
| AGAGGAAGATGGCTGTAGCTGCCGATTTCCAGAAGAAGAAGAAGGAGGATGTGA | |
| ACTGAGAGTGAAGTTCAGCAGGAGCGCAGACGCCCCCGCGTACCAGCAGGGCCA | |
| GAACCAGCTCTATAACGAGCTCAATCTAGGACGAAGAGAGGAGTACGATGTTTTG | |
| GACAAGAGACGTGGCCGGGACCCTGAGATGGGGGGAAAGCCGCAGAGAAGGAA | |
| GAACCCTCAGGAAGGCCTCTACAATGAACTGCAGAAAGATAAGATGGCGGAGGC | |
| CTACAGTGAGATTGGGATGAAAGGCGAGCGCCGGAGGGGCAAGGGGCACGATGG | |
| CCTTTACCAGGGTCTCAGTACAGCCACCAAGGACACCTACGACGCCCTTCACATG | |
| CAGGCCCTGCCCCCTCGCTAA | |
| SEQ ID NO: 19 | CD8 hinge NA |
| AAGCCCACCACGACGCCAGCGCCGCGACCACCAACACCGGCGCCCACCATCGCGT | |
| CGCAGCCCCTGTCCCTGCGCCCAGAGGCGAGCCGGCCAGCGGCGGGGGGCGCAG | |
| TGCACACGAGGGGGCTGGACTTCGCCAGTGAT | |
| SEQ ID NO: 20 | CD8 hinge AA |
| KPTTTPAPRPPTPAPTIASQPLSLRPEASRPAAGGAVHTRGLDFASD | |
| SEQ ID NO: 21 | CD8 TM NA |
| TTTTGGGTGCTGGTGGTGGTTGGTGGAGTCCTGGCTTGCTATAGCTTGCTAGTAAC | |
| AGTGGCCTTTATTATTTTCTGGGTGAGGAGTAAGAGGAGCAGGCTCCTGCACAGT | |
| GACTACATGAACATGACTCCCCGCCGCCCCGGGCCCACCCGCAAGCATTACCAGC | |
| CCTATGCCCCACCACGCGACTTCGCAGCCTATCGCTCC | |
| SEQ ID NO: 22 | CD8 TM AA |
| FWVLVVVGGVLACYSLLVTVAFIIFWVRSKRSRLLHSDYMNMTPRRPGPTRKHYQPY | |
| APPRDFAAYRS | |
| SEQ ID NO: 23 | CD3 zeta NA |
| AGAGTGAAGTTCAGCAGGAGCGCAGACGCCCCCGCGTACCAGCAGGGCCAGAAC | |
| CAGCTCTATAACGAGCTCAATCTAGGACGAAGAGAGGAGTACGATGTTTTGGACA | |
| AGAGACGTGGCCGGGACCCTGAGATGGGGGGAAAGCCGCAGAGAAGGAAGAACC | |
| CTCAGGAAGGCCTGTACAATGAACTGCAGAAAGATAAGATGGCGGAGGCCTACA | |
| GTGAGATTGGGATGAAAGGCGAGCGCCGGAGGGGCAAGGGGCACGATGGCCTTT | |
| ACCAGGGTCTCAGTACAGCCACCAAGGACACCTACGACGCCCTTCACATGCAGGC | |
| CCTGCCCCCTCGC | |
| SEQ ID NO: 24 | CD3 zeta AA |
| RVKFSRSADAPAYQQGQNQLYNELNLGRREEYDVLDKRRGRDPEMGGKPQRRKNP | |
| QEGLYNELQKDKMAEAYSEIGMKGERRRGKGHDGLYQGLSTATKDTYDALHMQAL | |
| PPR | |
| SEQ ID NO: 25 | 4-1BB NA |
| AAACGGGGCAGAAAGAA | |
| ACTCCTGTATATATTCAAACAACCATTTATGAGACCAGTACAAACTACTCAAGAG | |
| GAAGA TGGCTGTAGCTGCCGATTTCCAGAAGAAGAAGAAGGAGGATGTGAACTG | |
| SEQ ID NO: 26 | 4-1BB AA |
| KRGRKKLLYIFKQPFMRPVQTTQEEDGCSCRFPEEEEGGCEL | |
| SEQ ID NO: 27 | Short linker scFv |
| QVQLVESGGGVVQPGRSLRLSCAASGFTFSTYAMSWVRQAPGKGLEWVASISSGGNT | |
| YYPDSVKGRFTISRDNSKNTLYLQMNSLRAEDTAVYYCARDSYYFGNSVYYAMDY | |
| WGAGTTVTVSSGGGGSDIQMTQSPSSLSASVGDRVTITCKASQDINSYFSWYQQKPG | |
| KAPKLLIYRANRLVTGVPSRFSGSGSGTDFTFTISSLQPEDIATYYCLQYDEFPYTFGGG | |
| TRLEIK | |
| SEQ ID NO: 28 | Short linker scFv |
| CAGGTACAACTGGTGGAATCCGGCGGGGGAGTAGTACAGCCCGGACGATCTCTTC | |
| GACTCTCATGTGCAGCGTCCGGGTTCACTTTTTCTACCTACGCAATGTCATGGGTA | |
| CGACAGGCGCCGGGCAAAGGCCTCGAATGGGTTGCATCCATTTCATCAGGAGGTA | |
| ATACATATTATCCTGATTCAGTCAAGGGCCGATTCACGATTAGTCGAGATAATAGC | |
| AAGAACACTCTCTACTTGCAGATGAACTCCCTGCGGGCTGAGGACACGGCCGTGT | |
| ATTATTGCGCTCGCGATAGTTATTACTTCGGCAATTCCGTATATTATGCGATGGAC | |
| TATTGGGGCGCCGGTACTACCGTGACTGTTTCCTCTGGTGGAGGCGGATCCGACAT | |
| TCAAATGACCCAGTCTCCCTCAAGTTTGTCTGCATCTGTTGGCGATAGAGTTACAA | |
| TAACATGCAAAGCCAGTCAAGACATCAACTCATACTTCTCCTGGTATCAACAAAA | |
| GCCAGGAAAAGCTCCGAAACTGTTGATCTACCGGGCCAACCGGCTGGTCACTGGC | |
| GTGCCATCCCGGTTCAGTGGCAGCGGAAGCGGAACAGATTTCACGTTTACCATCT | |
| CTAGCCTCCAACCGGAGGACATCGCAACATACTATTGCCTTCAGTATGATGAGTTT | |
| CCCTACACTTTCGGTGGCGGCACCCGACTTGAGATCAAA | |
| SEQ ID NO: 29 CDR1 heavy chain | |
| TYA | |
| SEQ ID NO: 30 | CDR2 heavy chain |
| SSGGNT | |
| SEQ ID NO: 31 | CDR3 heavy chain |
| DSYYFGNSVYYAMDY | |
| SEQ ID NO: 32 | CDR1 light chain |
| QDINSY | |
| SEQ ID NO: 33 | CDR2 light chain |
| RAN | |
| SEQ ID NO: 34 | CDR3 light chain |
| LQYDEFPYT | |
| SEQ ID NO: 35 | CAR (long linker scFv) pCB7306 |
| MALPVTALLLPLALLLHAARPQVQLVESGGGVVQPGRSLRLSCAASGFTFSTYAMSW | |
| VRQAPGKGLEWVASISSGGNTYYPDSVKGRFTISRDNSKNTLYLQMNSLRAEDTAVY | |
| YCARDSYYFGNSVYYAMDYWGAGTTVTVSSGGGGSGGGGSGGGGSDIQMTQSPSSL | |
| SASVGDRVTITCKASQDINSYFSWYQQKPGKAPKLLIYRANRLVTGVPSRFSGSGSGT | |
| DFTFTISSLQPEDIATYYCLQYDEFPYTFGGGTRLEIKTTTPAPRPPTPAPTIASQPLSLRP | |
| EACRPAAGGAVHTRGLDFACDIYIWAPLAGTCGVLLLSLVITLYCKRGRKKLLYIFKQ | |
| PFMRPVQTTQEEDGCSCRFPEEEEGGCELRVKFSRSADAPAYQQGQNQLYNELNLGR | |
| REEYDVLDKRRGRDPEMGGKPRRKNPQEGLYNELQKDKMAEAYSEIGMKGERRRG | |
| KGHDGLYQGLSTATKDTYDALHMQALPPR | |
| SEQ ID NO: 36 | CAR (long linker scFv) |
| ATGGCGTTGCCCGTGACCGCACTTCTGCTTCCATTGGCACTCCTGCTCCACGCGGC | |
| AAGACCGCAGGTACAACTGGTGGAATCCGGCGGGGGAGTAGTACAGCCCGGACG | |
| ATCTCTTCGACTCTCATGTGCAGCGTCCGGGTTCACTTTTTCTACCTACGCAATGTC | |
| ATGGGTACGACAGGCGCCGGGCAAAGGCCTCGAATGGGTTGCATCCATTTCATCA | |
| GGAGGTAATACATATTATCCTGATTCAGTCAAGGGCCGATTCACGATTAGTCGAG | |
| ATAATAGCAAGAACACTCTCTACTTGCAGATGAACTCCCTGCGGGCTGAGGACAC | |
| GGCCGTGTATTATTGCGCTCGCGATAGTTATTACTTCGGCAATTCCGTATATTATG | |
| CGATGGACTATTGGGGCGCCGGTACTACCGTGACTGTTTCCTCTGGTGGGGGTGG | |
| GTCCGGGGGCGGTGGTTCAGGTGGAGGCGGATCCGACATTCAAATGACCCAGTCT | |
| CCCTCAAGTTTGTCTGCATCTGTTGGCGATAGAGTTACAATAACATGCAAAGCCAG | |
| TCAAGACATCAACTCATACTTCTCCTGGTATCAACAAAAGCCAGGAAAAGCTCCG | |
| AAACTGTTGATCTACCGGGCCAACCGGCTGGTCACTGGCGTGCCATCCCGGTTCA | |
| GTGGCAGCGGAAGCGGAACAGATTTCACGTTTACCATCTCTAGCCTCCAACCGGA | |
| GGACATCGCAACATACTATTGCCTTCAGTATGATGAGTTTCCCTACACTTTCGGTG | |
| GCGGCACCCGACTTGAGATCAAAACAACCACGCCTGCCCCTCGGCCACCCACGCC | |
| TGCACCCACTATCGCTTCCCAGCCACTCTCTCTTCGGCCGGAGGCTTGTCGCCCCG | |
| CAGCGGGAGGCGCGGTTCATACTCGCGGGCTGGACTTTGCTTGCGACATCTACAT | |
| CTGGGCACCGCTTGCCGGAACGTGCGGGGTCTTGCTGCTGTCCCTCGTTATTACTC | |
| TTTACTGCAAAAGAGGAAGAAAAAAGTTGCTGTATATTTTTAAGCAACCATTTAT | |
| GCGCCCGGTCCAAACTACGCAAGAGGAGGATGGATGTAGCTGCCGATTCCCCGAA | |
| GAAGAGGAGGGTGGTTGCGAACTGAGAGTGAAATTTAGCCGGTCTGCTGACGCTC | |
| CGGCCTATCAGCAAGGGCAAAACCAACTTTACAATGAGCTTAACCTGGGGAGGCG | |
| AGAGGAATATGATGTATTGGATAAGCGCCGAGGGAGGGACCCAGAGATGGGAGG | |
| AAAACCGAGGAGAAAAAACCCGCAAGAGGGGCTTTATAATGAACTGCAGAAAGA | |
| TAAGATGGCGGAGGCTTACAGCGAGATCGGGATGAAGGGAGAGAGACGCAGAGG | |
| GAAAGGCCACGACGGTCTCTACCAAGGCCTGAGTACGGCCACGAAAGATACATAC | |
| GATGCCCTCCATATGCAGGCCCTGCCACCGAGG | |
| SEQ ID NO: 37 | CAR (short linker scFv) pCB7339 |
| MALPVTALLLPLALLLHAARPQVQLVESGGGVVQPGRSLRLSCAASGFTFSTYAMSW | |
| VRQAPGKGLEWVASISSGGNTYYPDSVKGRFTISRDNSKNTLYLQMNSLRAEDTAVY | |
| YCARDSYYFGNSVYYAMDYWGAGTTVTVSSGGGGSDIQMTQSPSSLSASVGDRVTIT | |
| CKASQDINSYFSWYQQKPGKAPKLLIYRANRLVTGVPSRFSGSGSGTDFTFTISSLQPE | |
| DIATYYCLQYDEFPYTFGGGTRLEIKTTTPAPRPPTPAPTIASQPLSLRPEACRPAAGGA | |
| VHTRGLDFACDIYIWAPLAGTCGVLLLSLVITLYCKRGRKKLLYIFKQPFMRPVQTTQ | |
| EEDGCSCRFPEEEEGGCELRVKFSRSADAPAYQQGQNQLYNELNLGRREEYDVLDKR | |
| RGRDPEMGGKPRRKNPQEGLYNELQKDKMAEAYSEIGMKGERRRGKGHDGLYQGL | |
| STATKDTYDALHMQALPPR | |
| SEQ ID NO: 38 | CAR (short linker scFv) |

ATGGCGTTGCCCGTGACCGCACTTCTGCTTCCATTGGCACTCCTGCTCCACGCGGC
AAGACCGCAGGTACAACTGGTGGAATCCGGCGGGGGAGTAGTACAGCCCGGACG
ATCTCTTCGACTCTCATGTGCAGCGTCCGGGTTCACTTTTTCTACCTACGCAATGTC
ATGGGTACGACAGGCGCCGGGCAAAGGCCTCGAATGGGTTGCATCCATTTCATCA
GGAGGTAATACATATTATCCTGATTCAGTCAAGGGCCGATTCACGATTAGTCGAG
ATAATAGCAAGAACACTCTCTACTTGCAGATGAACTCCCTGCGGGCTGAGGACAC
GGCCGTGTATTATTGCGCTCGCGATAGTTATTACTTCGGCAATTCCGTATATTATG
CGATGGACTATTGGGGCGCCGGTACTACCGTGACTGTTTCCTCTGGTGGAGGCGG
ATCCGACATTCAAATGACCCAGTCTCCCTCAAGTTTGTCTGCATCTGTTGGCGATA
GAGTTACAATAACATGCAAAGCCAGTCAAGACATCAACTCATACTTCTCCTGGTA
TCAACAAAAGCCAGGAAAAGCTCCGAAACTGTTGATCTACCGGGCCAACCGGCTG
GTCACTGGCGTGCCATCCCGGTTCAGTGGCAGCGGAAGCGGAACAGATTTCACGT
TTACCATCTCTAGCCTCCAACCGGAGGACATCGCAACATACTATTGCCTTCAGTAT
GATGAGTTTCCCTACACTTTCGGTGGCGGCACCCGACTTGAGATCAAAACAACCA
CGCCTGCCCCTCGGCCACCCACGCCTGCACCCACTATCGCTTCCCAGCCACTCTCT
CTTCGGCCGGAGGCTTGTCGCCCCGCAGCGGGAGGCGCGGTTCATACTCGCGGGC
TGGACTTTGCTTGCGACATCTACATCTGGGCACCGCTTGCCGGAACGTGCGGGGTC
TTGCTGCTGTCCCTCGTTATTACTCTTTACTGCAAAAGAGGAAGAAAAAAGTTGCT
GTATATTTTTAAGCAACCATTTATGCGCCCGGTCCAAACTACGCAAGAGGAGGAT
GGATGTAGCTGCCGATTCCCCGAAGAAGAGGAGGGTGGTTGCGAACTGAGAGTG
AAATTTAGCCGGTCTGCTGACGCTCCGGCCTATCAGCAAGGGCAAAACCAACTTT
ACAATGAGCTTAACCTGGGGAGGCGAGAGGAATATGATGTATTGGATAAGCGCCG
AGGGAGGGACCCAGAGATGGGAGGAAAACCGAGGAGAAAAAACCCGCAAGAGG
GGCTTTATAATGAACTGCAGAAAGATAAGATGGCGGAGGCTTACAGCGAGATCGG
GATGAAGGGAGAGAGACGCAGAGGGAAAGGCCACGACGGTCTCTACCAAGGCCT
GAGTACGGCCACGAAAGATACATACGATGCCCTCCATATGCAGGCCCTGCCACCG
AGG
SEQ ID NO: 39 STAT3 motif YXXQ
SEQ ID NO: 40 STAT3 motif YRHQ
SEQ ID NO: 41 STAT5 motif YXXL
SEQ ID NO: 42 STAT5 motif YLSL
SEQ ID NO: 43 JAK motif LKCNTPDPS
SEQ ID NO: 44 IL36γ-IL2SP
MYRMQLLSCIALSLALVTNSSMCKPITGTINDLNQQVWTLQGQNLVAVPRSDSVTPV
TVAVITCKYPEALEQGRGDPIYLGIQNPEMCLYCEKVGEQPTLQLKEQKIMDLYGQPE
PVKPFLFYRAKTGRTSTLESVAFPDWFIASSKRDQPIILTSELGKSYNTAFELNIND
SEQ ID NO: 45 IL36γ-IL2SP
ATGTACCGCATGCAACTTCTGTCTTGTATTGCGCTTTCTCTCGCACTCGTAACCAAT
TCCAGCATGTGTAAGCCGATAACCGGGACTATAAACGATTTGAATCAGCAGGTAT
GGACCTTGCAGGGACAGAATCTGGTAGCTGTACCACGGAGCGACAGCGTCACCCC
CGTGACTGTGGCCGTCATAACATGCAAATACCCGGAAGCGCTCGAACAGGGCAGG
GGTGATCCGATATACCTGGGAATCCAAAACCCGGAGATGTGCCTTTACTGCGAGA
AGGTGGGCGAACAACCTACGCTTCAATTGAAAGAGCAAAAGATAATGGACCTGTA
CGGCCAACCCGAACCCGTAAAGCCCTTCCTCTTTTACCGCGCCAAGACTGGTAGA
ACAAGTACTCTGGAGAGCGTTGCTTTTCCTGATTGGTTTATAGCAAGCTCCAAAAG
GGACCAGCCGATTATCCTCACAAGCGAACTCGGAAAGTCCTATAATACCGCTTTT
GAGTTGAATATCAATGAC
SEQ ID NO: 46 IL36γ-IL36SP
MRGTPGDADGGGRAVYQSMCKPITGTINDLNQQVWTLQGQNLVAVPRSDSVTPVTV
AVITCKYPEALEQGRGDPIYLGIQNPEMCLYCEKVGEQPTLQLKEQKIMDLYGQPEPV
KPFLFYRAKTGRTSTLESVAFPDWFIASSKRDQPIILTSELGKSYNTAFELNIND
SEQ ID NO: 47 IL36γ-IL36SP
ATGAGAGGGACTCCCGGAGACGCAGACGGTGGAGGCCGAGCTGTTTATCAAAGC
ATGTGTAAGCCGATAACCGGGACTATAAACGATTTGAATCAGCAGGTATGGACCT
TGCAGGGACAGAATCTGGTAGCTGTACCACGGAGCGACAGCGTCACCCCCGTGAC
TGTGGCCGTCATAACATGCAAATACCCGGAAGCGCTCGAACAGGGCAGGGGTGAT
CCGATATACCTGGGAATCCAAAACCCGGAGATGTGCCTTTACTGCGAGAAGGTGG
GCGAACAACCTACGCTTCAATTGAAAGAGCAAAAGATAATGGACCTGTACGGCCA
ACCCGAACCCGTAAAGCCCTTCCTCTTTTACCGCGCCAAGACTGGTAGAACAAGT
ACTCTGGAGAGCGTTGCTTTTCCTGATTGGTTTATAGCAAGCTCCAAAAGGGACCA
GCCGATTATCCTCACAAGCGAACTCGGAAAGTCCTATAATACCGCTTTTGAGTTGA
ATATCAATGAC
SEQ ID NO: 48 IL36γ-IL2 mutant SP
MRRMQLLLLIALSLALVTNSSMCKPITGTINDLNQQVWTLQGQNLVAVPRSDSVTPV
TVAVITCKYPEALEQGRGDPIYLGIQNPEMCLYCEKVGEQPTLQLKEQKIMDLYGQPE
PVKPFLFYRAKTGRTSTLESVAFPDWFIASSKRDQPIILTSELGKSYNTAFELNIND
SEQ ID NO: 49 IL36γ-IL2 mutant SP
ATGCGGCGCATGCAACTTCTGCTGCTCATTGCGCTTTCTCTCGCACTCGTAACCAA
TTCCAGCATGTGTAAGCCGATAACCGGGACTATAAACGATTTGAATCAGCAGGTA
TGGACCTTGCAGGGACAGAATCTGGTAGCTGTACCACGGAGCGACAGCGTCACCC
CCGTGACTGTGGCCGTCATAACATGCAAATACCCGGAAGCGCTCGAACAGGGCAG
GGGTGATCCGATATACCTGGGAATCCAAAACCCGGAGATGTGCCTTTACTGCGAG
AAGGTGGGCGAACAACCTACGCTTCAATTGAAAGAGCAAAAGATAATGGACCTGT
ACGGCCAACCCGAACCCGTAAAGCCCTTCCTCTTTTACCGCGCCAAGACTGGTAG
AACAAGTACTCTGGAGAGCGTTGCTTTTCCTGATTGGTTTATAGCAAGCTCCAAAA
GGGACCAGCCGATTATCCTCACAAGCGAACTCGGAAAGTCCTATAATACCGCTTT
TGAGTTGAATATCAATGAC
SEQ ID NO: 50 IL-2Rb
NCRNTGPWLKKVLKCNTPDPSKFFSQLSSEHGGDVQKWLSSPFPSSSFSPGGLAPEISP
LEVLERDKVTQLLPLNTDAYLSLQELQGQDPTHLV
SEQ ID NO: 51 IL-2Rb
AACTGCCGAAACACCGGACCCTGGCTCAAAAAGGTTCTTAAATGCAACACTCCTG
ACCCATCCAAGTTTTTTTCACAATTGTCATCAGAGCACGGAGGCGATGTACAAAA
GTGGCTTAGTTCCCCTTTCCCGAGCTCCTCCTTTAGTCCAGGGGGTCTCGCCCCTG
AAATTAGTCCCCTCGAAGTTTTGGAGAGAGATAAGGTGACCCAATTGCTCCCACT
GAACACCGACGCATATCTCTCCTTGCAAGAGTTGCAAGGCCAGGACCCCACCCAC
CTGGTC

1. A T cell comprising a chimeric antigen receptor (CAR) comprising:
(i) an anti-ROR1 scFv wherein the anti-ROR1 scFv comprises a light chain (V_{L}) and a heavy chain (V_{H}), and the V_{L} comprises a sequence selected from SEQ ID NOs: 3, 7, and 11, the V_{H} comprises a sequence selected from SEQ ID NOs: 2, 6, and 10;
(ii) a transmembrane domain;
(iii) a hinge domain
(iv) a cytoplasmic domain comprising a CD3 zeta domain and a 4-1BB domain.

2-3. (canceled)

4. The T cell of claim 1, wherein the anti-ROR1 scFv comprises a light chain (V_{L}) and a heavy chain (V_{H}), and the V_{L} consists essentially of a sequence selected from SEQ ID NOs: 3, 7, and 11, and the V_{H} consists essentially of a sequence selected from SEQ ID NOs: 2, 6, and 10.

5. The T cell of claim 1, wherein the anti-ROR1 scFv comprises a linker linking the light chain (V_{L}) and the heavy chain (V_{H}), wherein the linker comprises a formula (GₓS_{y})ₙ, where G is glycine and S is serine.

6-8. (canceled)

9. The T cell of claim 1, wherein the anti-ROR1 scFv comprises complementarity determining regions CDR1, CDR2, and CDR3 in the light chain (V_{L}), and CDR1, CDR2, and CDR3 in the heavy chain (V_{H}) and comprises: SEQ ID NO: 29 in the CDR1 of the V_{H}, SEQ ID NO: 30 in the CDR2 of the V_{H}, SEQ ID NO: 31 in the CDR3 of the V_{H}, SEQ ID NO: 32 in the CDR1 of the V_{L}, SEQ ID NO: 33 in the CDR2 of the V_{L}, and SEQ ID NO: 34 in the CDR3 of the V_{L}.

10-11. (canceled)

12. The T cell of claim 1, wherein the transmembrane domain comprises a CD8 transmembrane domain consisting essentially of SEQ ID NO: 22 and encoded by a nucleic acid consisting essentially of SEQ ID NO: 21, and the hinge domain comprises a CD8 hinge domain consisting essentially of SEQ ID NO: 20 and encoded by a nucleic acid consisting essentially of SEQ ID NO: 19.

13-17. (canceled)

18. The T cell of claim 1, wherein the CD3 zeta domain consists essentially of SEQ ID NO. 24 and is encoded by a nucleic acid consisting essentially of SEQ ID NO. 23, and wherein the 4-1BB domain consists essentially of SEQ ID NO. 26 and is encoded by a nucleic acid consisting essentially of SEQ ID NO. 25.

19-23. (canceled)

24. The T cell of claim 1, wherein the cytoplasmic domain comprises a binding motif for an intracellular signal transduction protein selected from a STAT protein and a JAK protein, wherein the JAK binding domain comprises SEQ ID NO: 43 and the STAT binding domain is selected from SEQ ID NO: 39-42.

25-28. (canceled)

29. The T cell of claim 1, wherein the cytoplasmic domain further comprises an IL-2Rb cytoplasmic domain.

30. The T cell of claim 29, wherein the IL-2Rb cytoplasmic domain consists essentially of SEQ ID NO: 50 and is encoded by a nucleic acid consisting essentially of SEQ ID NO. 51.

31. (canceled)

32. The T cell of claim 1, wherein the CAR comprises the sequence selected from SEQ ID NOs: 4, 8, 12, and 27.

33-49. (canceled)

50. The T cell of claim 1, further comprising. an inactivation of an immune checkpoint gene or a regulatory gene selected from the group consisting of PDCD1, CTLA-4, LAG3, Tim3, BTLA, BY55, TIGIT, B7H5, LAIR1, SIGLEC10, B2M, CISH, CBLB and 2B4.

51-58. (canceled)

59. The T cell of claim 50, comprising inactivation of B2M and further comprising insertion of an HLA-E-B2M fusion construct into the B2M gene.

60-65. (canceled)

66. A method of making the T cell of claim 47, the method comprising introducing into a cell a nucleic acid comprising a sequence selected from SEQ ID NOs: 14, 16, 18, 36, and 38.

67-68. (canceled)

69. The method of claim 66, wherein the introducing step comprises introducing into the cell a sequence-dependent endonuclease comprising a CRISPR system comprising a nucleic acid-guided endonuclease and nucleic acid-targeting nucleic acid (NATNA) guides.

70-73. (canceled)

74. The method of claim 69, wherein the endonuclease cleaves the genome of the cell at a locus selected from the group consisting of TRAC, CBLB, PDCD1, CTLA-4, LAG3, Tim3, BTLA, BY55, TIGIT, B7H5, LAIR1, SIGLEC10, 2B4, and B2M.

75-135. (canceled)
